(19) United States
(12) Patent Application Publication
(10) Pub. No.: US 2024/0228521 A1
(43) Pub. Date: Jul. 11, 2024

(54) ANTI-VIRAL AND ANTI-TUMORAL COMPOUNDS, AND USES THEREOF

(71) Applicant: YEDA RESEARCH AND DEVELOPMENT CO. LTD., Rehovot (IL)

(72) Inventors: David SPERANDIO, Rehovot (IL); Helena SHOMAR MONNGES, Rehovot (IL); Eugene J. EISENBERG, Rehovot (IL); Arthur MACHLENKIN, Rehovot (IL)

(73) Assignee: YEDA RESEARCH AND DEVELOPMENT CO. LTD., Rehovot (IL)

(21) Appl. No.: 18/554,795

(22) PCT Filed: Apr. 14, 2022
(86) PCT No.: PCT/IL2022/050395

§ 371 Date: Oct. 11, 2023

Related U.S. Application Data
(60) Provisional application No. 63/174,549, filed on Apr. 14, 2021.

(51) Int. Cl. C07F 9/6561; A61K 31/675

(52) U.S. Cl. CPC C07F 9/6561; A61K 31/675

(57) ABSTRACT

Disclosed herein are 3, 4-didehydro- and 3′-deoxy-3, 4-didehydro-compounds, and pharmaceutical compositions thereof. The 3, 4-didehydro- and 3′-deoxy-3, 4-didehydro-compounds, and pharmaceutical compositions thereof may be used in methods of treating diseases including virus-induced diseases, cancer, autoimmune diseases, immune disorders, and bacterial-associated diseases or infections, or combinations thereof. Examples of viral-induced diseases include viral infections by RNA or DNA viruses, for example SARS-CoV-2, EBV, and BKV.

### FIELD OF INTEREST

Disclosed herein are 3, 4-didehydro- and 3′-deoxy-3, 4-didehydro-compounds having the structure of Formula A

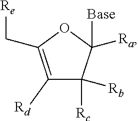

pharmaceutical salts thereof, and pro-drugs thereof, wherein the compounds comprise natural and non-natural bases. Further disclosed are pharmaceutical compositions thereof, and uses thereof. Uses of the compounds and compositions disclosed herein include as anti-viral, anti-bacterial, and anti-cancer therapeutic agents.

### BACKGROUND

Nucleotide/nucleoside analogs are crucial components of our medicinal chemistry arsenal, with more than 30 approved molecules in the market and many more currently in development. They are currently employed to treat a wide array of pathologies, including viral and microbial infections, as well as to inhibit the proliferation of cancer cells. Moreover, it is known that minor changes in their chemical structure have profound effects on their activity against specific targets, as well as on potential undesired side-effects.

One example of a nucleotide analog with therapeutically relevant activity is ddhCTP, which includes a 3, 4-didehydro-ribose structure and acts as a chain terminator for certain viral RNA-dependent RNA polymerases, leading to inhibition of viral replication. Substituted nucleotide/nucleoside analog compounds are thought to mimic the overall structure of nucleotide/nucleosides and so may provide unique novel activities. Accordingly, substituted nucleotide and nucleoside analog compounds, for example, substituted ddh- or deoxy-ddh-compounds comprising the structure of Formula A

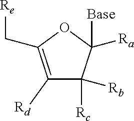

may add unique therapeutic compounds to the medicinal chemistry arsenal.

These synthetic analog compounds may mimic the overall structure of nucleotide/nucleoside analogs and may include: (1) natural and non-naturally occurring bases attached to position C1′ of the 5-membered ring (to the ddh- or deoxy-ddh-ribose sugar analog) and/or (2) different substitutions on the 5-member ribose sugar (at positions C2′, C3′, and/or C5′); and may comprise unique novel activities. In some embodiments, the compounds include C- and N-linked nucleosides. Prodrugs of these substituted compounds may be synthesized and used in methods of treating a disease in a subject in need, wherein the prodrug may be converted into an active compound or metabolite thereof once in the cells as a result of spontaneous chemical reaction(s), enzyme catalyzed chemical reaction(s), photolysis, and/or metabolic chemical reaction(s).

There remains a need for targeted active compounds for treating diseases in a subject including viral and microbial infections, as well as to inhibit the proliferation of cancer cells. Thus, there is a need for creating novel structural modifications of existing compounds to generate new therapeutic variants with selective antiviral, antimicrobial, or anti-cancer activities, or a combination of activities thereof, and with optimal intracellular levels of the active triphosphate compounds for optimized therapeutic activities.

The present disclosure solves this need by providing novel synthetic ddh- and deoxy-ddh-compounds, for therapeutic use as antiviral, anti-tumoral, and/or antibacterial agents.

### SUMMARY OF THE DISCLOSURE

In one aspect, provided herein is a ddh- or a deoxy-ddh-compound represented by the structure of Formula I

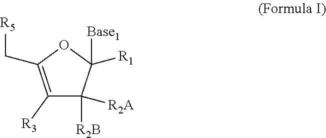

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl; R₅ is

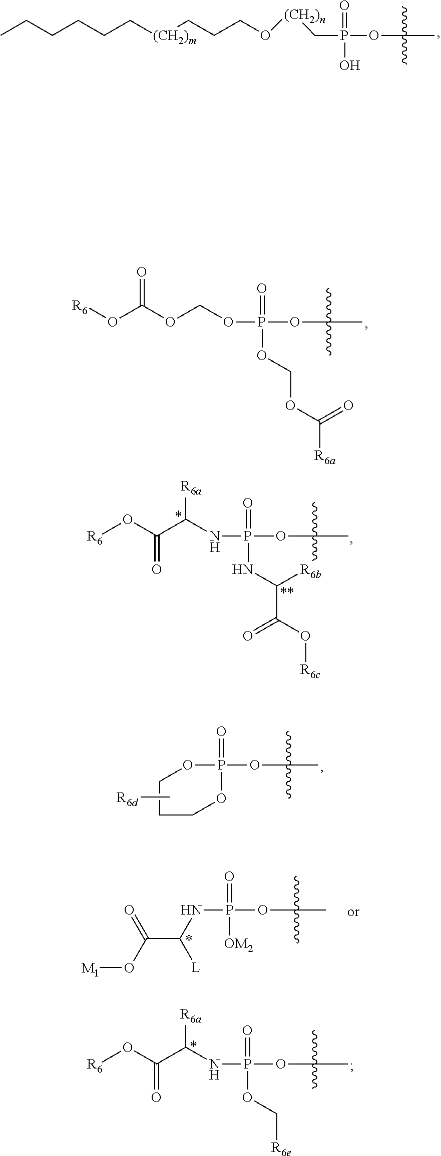

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₁ is an N-linked natural base.

In one aspect, provided herein is a ddh- or a deoxy-ddh-compound represented by the structure of Formula II

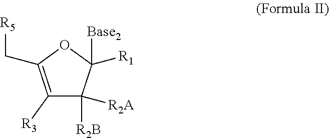

or a pharmaceutically acceptable salt thereof,
wherein
R₁, R₂A, R₂B, R₃, R₅, R₆, R₆ₐ, R_{6b}, R_{6c}, R_{6d}, R₆ₑ, R₈, R₉, R₁₀, n, and m are as defined in Formula I; and
Base₂ is an N-linked modified purine or pyrimidine base.

In one aspect, a ddh- or a deoxy-ddh-compound represented by the structure of Formula III

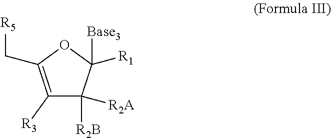

or a pharmaceutically acceptable salt thereof,
wherein
R₁, R₂A, R₂B, R₃, R₅, R₆, R₆ₐ, R_{6b}, R_{6c}, R_{6d}, R₆ₑ, R₈, R₉, R₁₀, n, and m are as defined in Formula I;
wherein if R₃ is H, then R₁ is —N₃, an alkyne-R₈, or an alkyl;
and
Base₃ is a C-linked base.

In one aspect, provided herein is a ddh- or a deoxy-ddh-compound represented by the structure of Formula IV

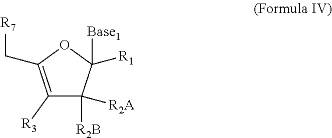

or a pharmaceutically acceptable salt thereof,
wherein
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is halo, H or an alkyl;
wherein:
when R₃ is a halo, then R₁ is H, —CN, —N₃, an alkyne-R₈, or an alkyl;
when R₃ is H or an alkyl, then R₁ is —N₃, an alkyne-R₈, or an alkyl;
R₇ is OH,

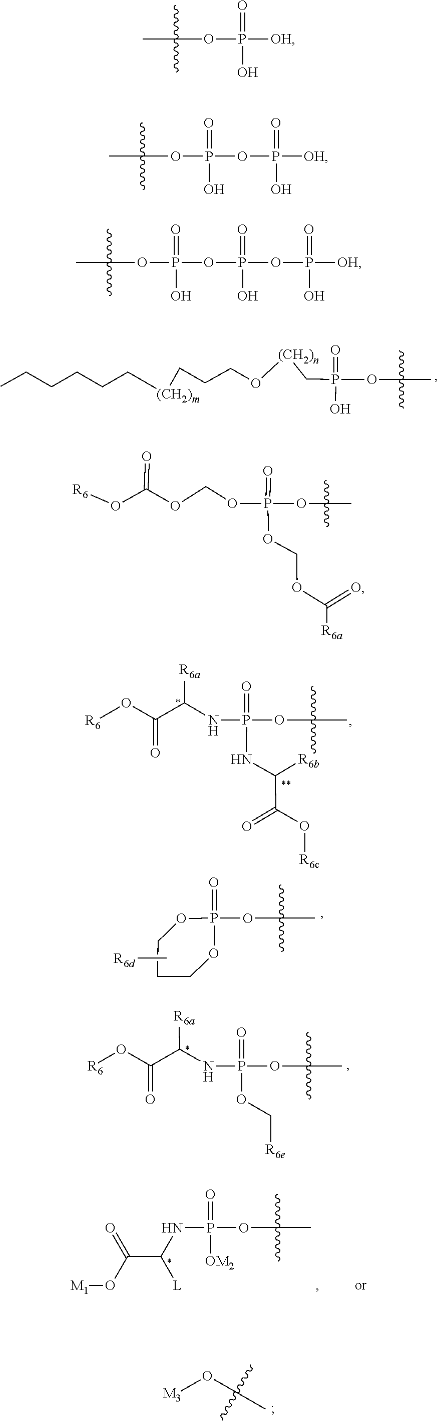

M₁ is a branched or linear alkyl;
L is a side chain of a natural or unnatural amino acid;
M₂ is substituted or unsubstituted aryl, or substituted or unsubstituted heteroaryl;
M₃ is

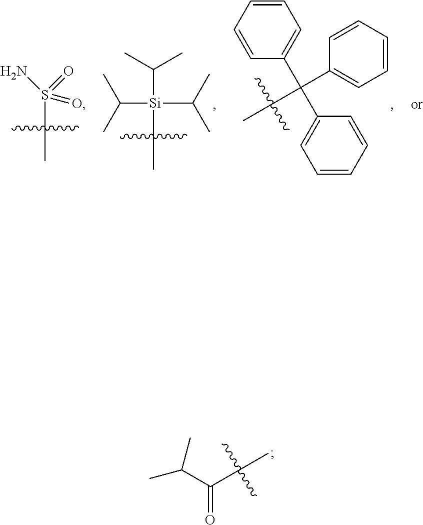

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₁ is an N-linked natural base.

In one aspect, provided herein is a ddh- or a deoxy-ddh-compound represented by the structure of Formula V

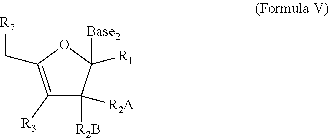

or a pharmaceutically acceptable salt thereof,
wherein
R₁, R₂A, R₂B, R₃, R₆, R₆ₐ, R_{6b}, R_{6c}, R_{6d}, R₆ₑ, R₇, R₈, R₉, R₁₀, n, m, M₁, M₂, M₃ and L are as defined in Formula IV; and
Base₂ is an N-linked modified purine or pyrimidine base.

In one aspect, provided herein is a ddh- or a deoxy-ddh-compound represented by the structure of Formula VI

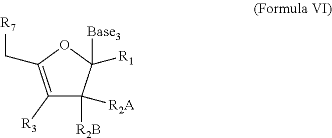

or a pharmaceutically acceptable salt thereof,
wherein
R₁, R₂A, R₂B, R₃, R₆, R₆ₐ, R_{6b}, R_{6c}, R_{6d}, R₆ₑ, R₇, R₈, R₉, R₁₀, n, m, M₁, M₂, M₃ and L are as defined in Formula IV; and
Base₃ is a C-linked base.

In one aspect, provided herein is a ddh- or a deoxy-ddh-compound represented by the structure of Formula VII

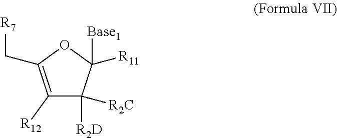

or a pharmaceutically acceptable salt thereof,
wherein
R₁₁ is H or —CN;
R₂C is an alkyl or an alkyne-R₉;
R₂D is an alkyl or an alkyne-R₁₀;
R₁₂ is H or an alkyl;
R₇ is OH,

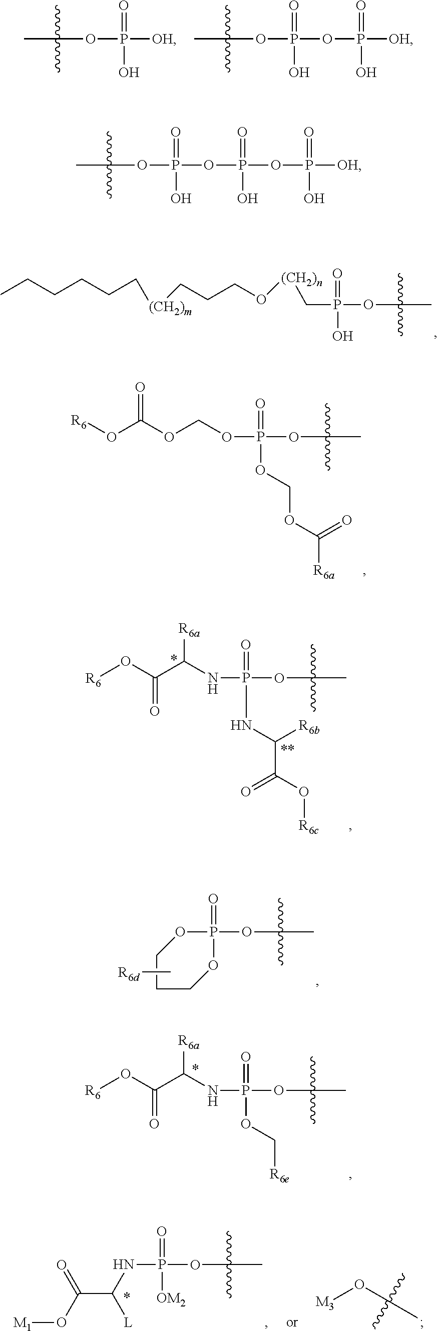

M₁ is a branched or linear alkyl;
L is a side chain of a natural or unnatural amino acid;
M₂ is substituted or unsubstituted aryl, or substituted or unsubstituted heteroaryl;
M₃ is

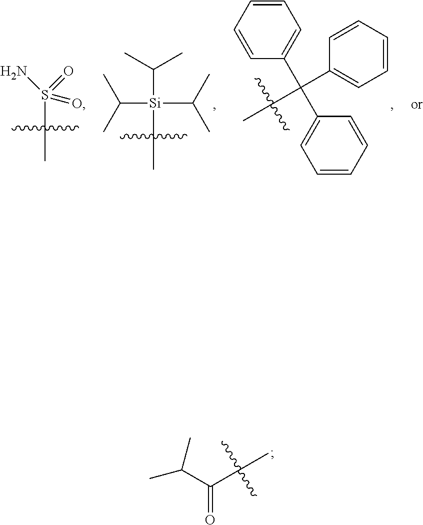

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₉, and R₁₀ are each independently an alkyl; and
Base₁ is an N-linked natural base.

In one aspect, provided herein is a ddh- or a deoxy-ddh-compound represented by the structure of Formula VIII

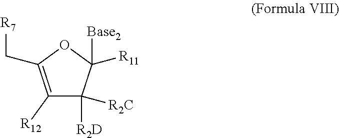

or a pharmaceutically acceptable salt thereof,
wherein
R₁₁, R₂C, R₂D, R₁₂, R₆, R₆ₐ, R_{6b}, R_{6c}, R_{6d}, R₆ₑ, R₇, R₉, R₁₀, n, m, M₁, M₂, M₃ and L are as defined in Formula VII; and
Base₂ is an N-linked modified purine or pyrimidine base.

In one aspect, provided herein is ddh- or a deoxy-ddh-compound
represented by the structure of Formula IX

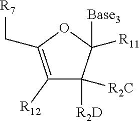

or a pharmaceutically acceptable salt thereof,
wherein
R₁₁, R₂C, R₂D, R₁₂, R₆, R₆ₐ, R_{6b}, R_{6c}, R_{6d}, R₆ₑ, R₇, R₉, R₁₀, n, m, M₁, M₂, M₃ and L are as defined in Formula VII; and
Base₃ is C-linked base.

In one aspect, provided herein is ddh- or a deoxy-ddh-compound represented by the structure of Formula X

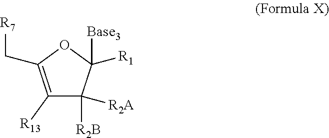

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₁₃ is an alkyl(C1-C6);
R₇ is OH,

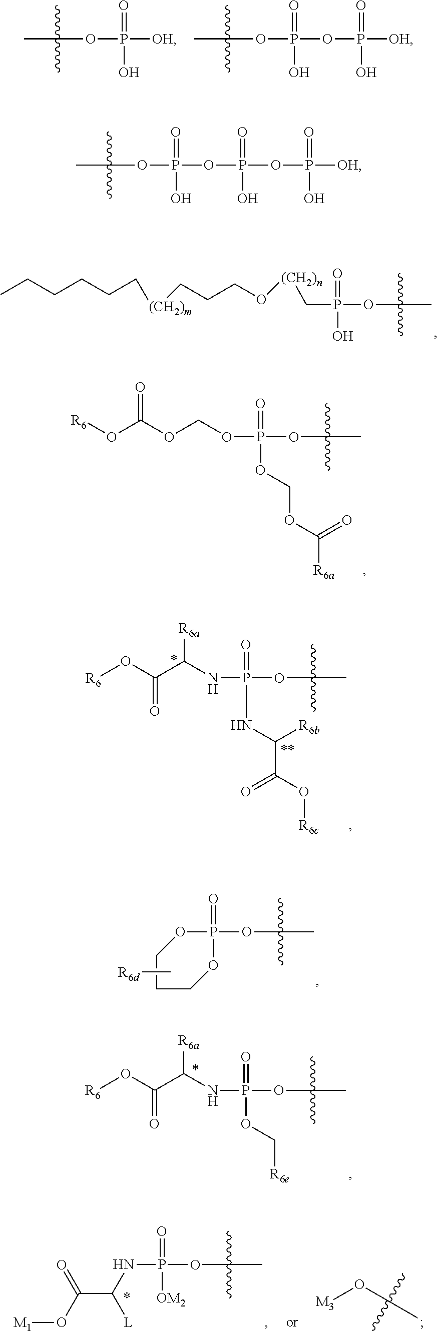

M₁ is a branched or linear alkyl;
L is a side chain of a natural or unnatural amino acid;
M₂ is substituted or unsubstituted aryl;
M₃ is

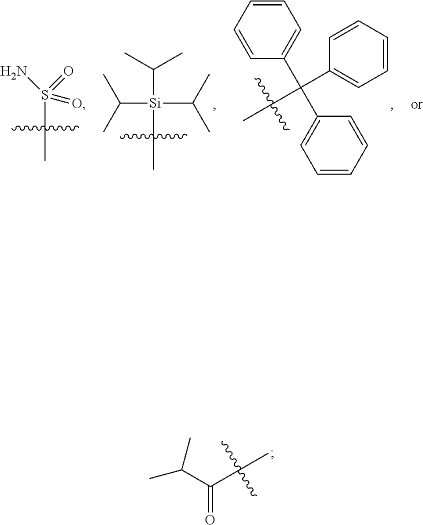

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₃ is a C-linked base.

In one aspect, Base₁ of Formula I, IV, and VII is N-linked natural base wherein the N-linked natural base comprises

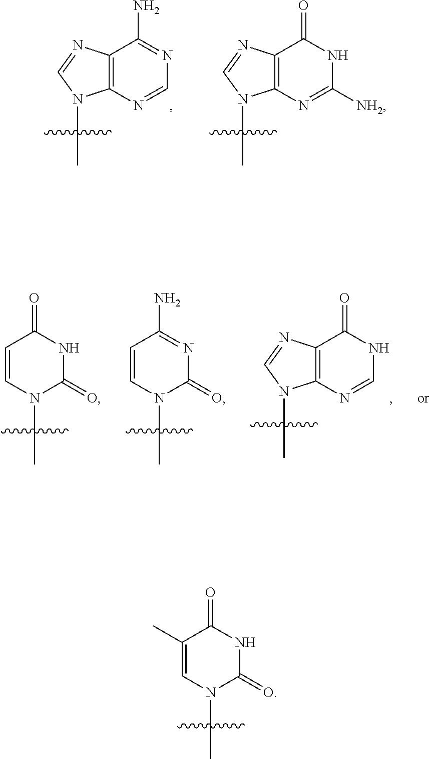

In one aspect, Base₂ of Formula II, V, and VIII is an N-linked modified purine or pyrimidine base, wherein the N-linked modified purine or pyrimidine base comprises:

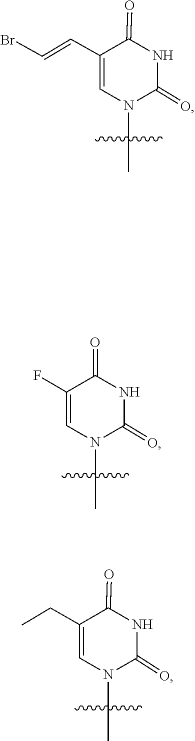 [SMILES: *n1cc(/C=C/Br)c(=O)[nH]c1=O ; *n1cc(F)c(=O)[nH]c1=O ; *n1cc(CC)c(=O)[nH]c1=O]

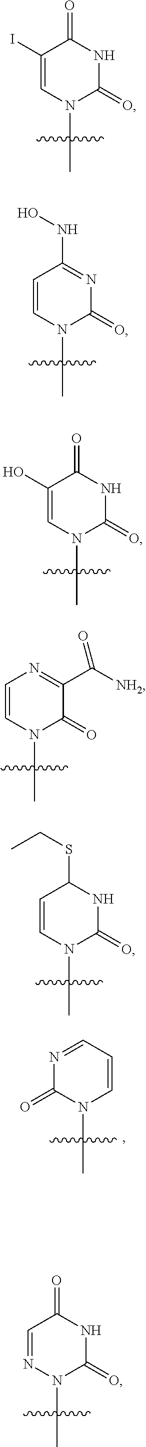

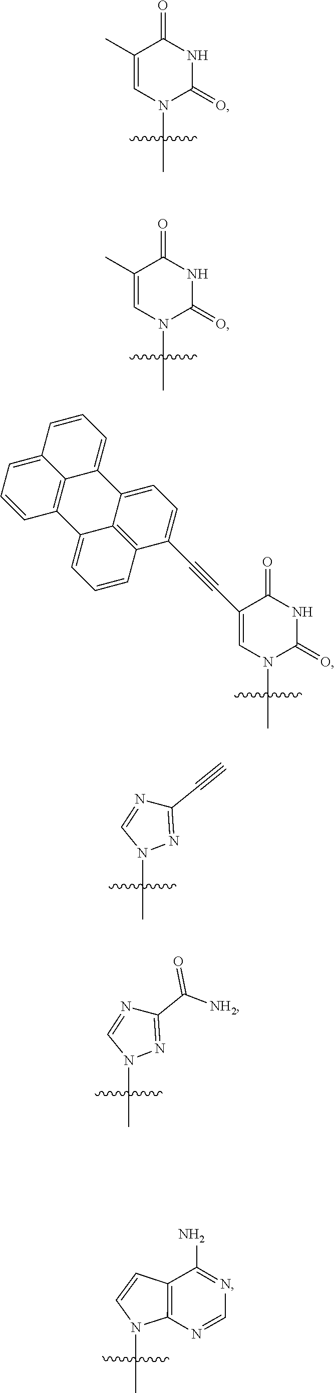

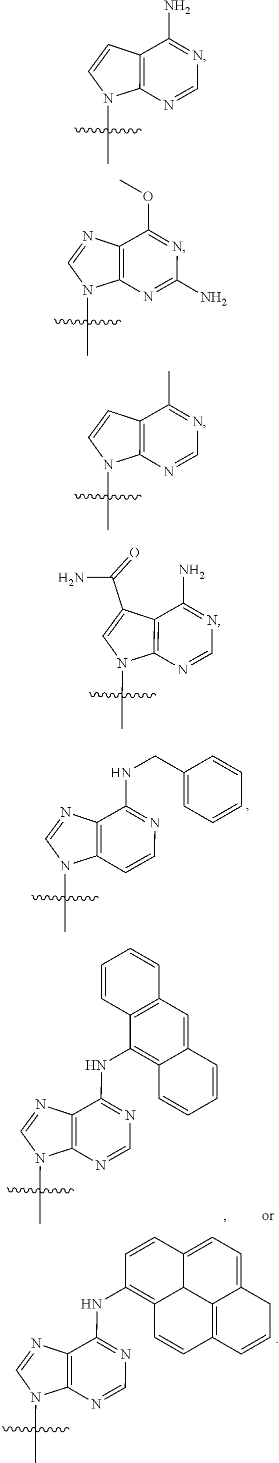

In one aspect, Base₃ of Formula III, VI, IX, and X is a C-linked base, wherein the C-linked base comprises:

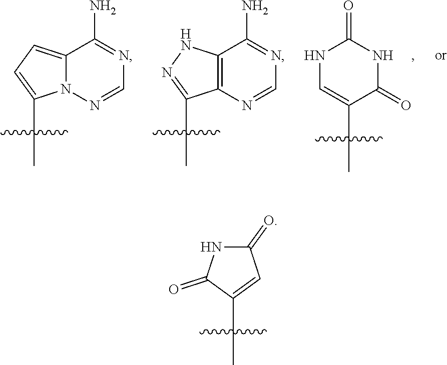

In one aspect, provided herein is a pharmaceutical composition comprising a compound disclosed herein.

In one aspect, provided herein is a pharmaceutical composition comprising at least two compounds disclosed herein.

In one aspect, provided herein is a pharmaceutical composition comprising a compound disclosed herein, for use in the treatment of a disease in a subject in need thereof.

In a related aspect, the disease comprises a virus-induced disease, a cancer, an autoimmune disease, an immune disorder, a bacterial associated disease or infection, or a combination thereof. In a further related aspect, the disease is caused by a virus selected from the group consisting of norovirus, rotavirus, hepatitis virus A, B, C, D, or E, rabies virus, West Nile virus, enterovirus, echovirus, coxsackievirus, herpes simplex virus (HSV), varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus (MERS-CoV), SARS coronavirus 2 (SARS-CoV-2), Epstein-Barr virus (EBV), influenza virus, influenza virus A2, influenza virus B, influenza virus A (H1N1), respiratory syncytial virus (RSV), polyoma viruses, BK virus, JC virus, Tacaribe virus, Ebola virus, Dengue virus, Pneumoviridae virus, respiratory syncytial virus, human metapneumovirus, Picornaviridae virus, human rhinovirus, Flaviviridae virus, yellow fever virus, hepatitis C virus (HCV), and Filoviridae virus, and any combination thereof. In another further related aspect, the disease is COVID-19 caused by SARS-CoV-2.

In another related aspect of treating a disease disclosed herein, the disease is a virus-induced disease. In a further related aspect, the virus-induced disease is a Pneumoviridae virus infection. In yet a further related aspect, a Pneumoviridae virus infection comprises a respiratory syncytial virus infection or a human metapneumovirus infection. In a further related aspect, the virus-induced disease is a Picornaviridae virus infection. In yet a further related aspect, the Picornaviridae virus infection comprises a human rhinovirus infection. In a further related aspect, the virus-induced disease is a Flaviviridae virus infection. In a further related aspect, the Flaviviridae virus infection comprises a dengue virus infection, a yellow fever virus infection, a West Nile virus infection, a zika virus infection, or a hepatitis C virus infection. In a further related aspect, the virus-induced disease is a Filoviridae virus infection. In yet a further related aspect, the Filoviridae virus infection comprises an Ebola virus infection.

In another related aspect, the method of use terminates polynucleotide chain synthesis in a cell. In a further related aspect, terminating polynucleotide chain synthesis increases termination of DNA chain synthesis, or increases termination of RNA chain synthesis, or a combination thereof. In another further related aspect, terminating polynucleotide chain synthesis confers viral resistance to said cell. In yet another further related aspect, the cell is a eukaryotic cell. In still another further related aspect, the eukaryotic cell is a tumor cell. In still another further related aspect, the eukaryotic cell is a eukaryotic cell infected by a virus or a foreign DNA.

### BRIEF DESCRIPTION OF THE DRAWINGS

The subject matter disclosed describing the synthetic ddh- and deoxy-ddh-compounds, and uses thereof, is particularly pointed out and distinctly claimed in the concluding portion of the specification. The compounds, synthesis of, and use thereof, however, both as to organization and method of operation, together with objects, features, and advantages thereof, may best be understood by reference to the following detailed description when read with the accompanying drawings.

FIG. 1 presents one embodiment of a synthetic scheme for the preparation of a compound described herein, e.g., Compound 16.

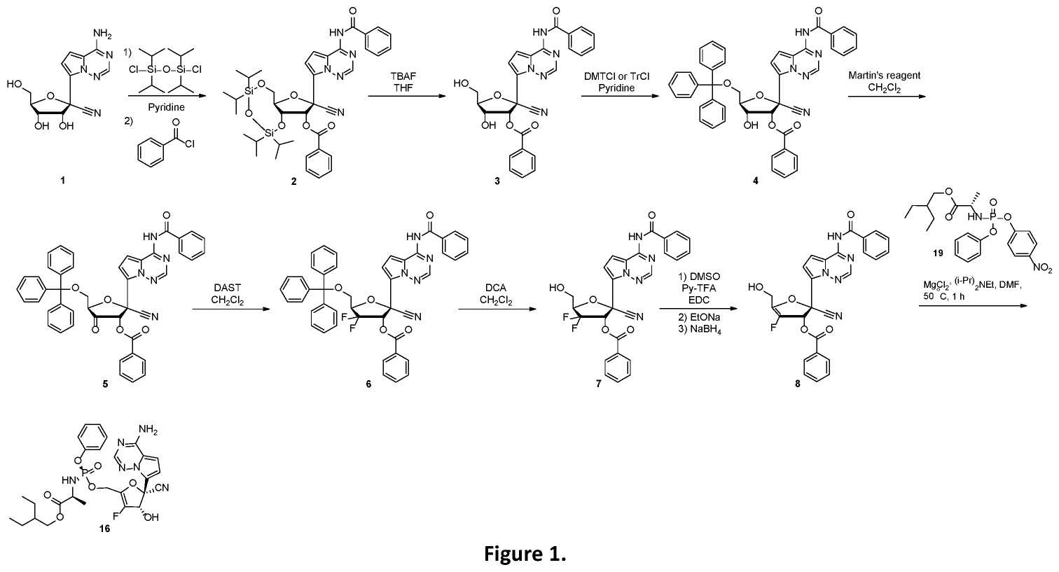

FIG. 2 presents another embodiment of a synthetic scheme for the preparation of compounds described herein, e.g., Compound 14 and Compound 15.

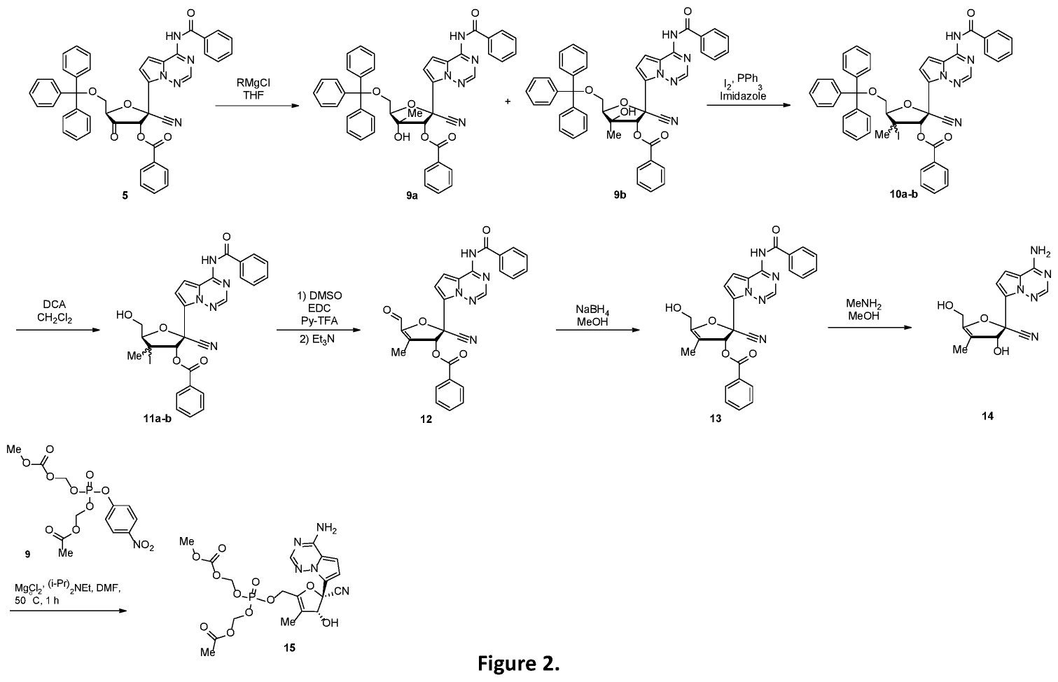

### DETAILED DESCRIPTION

In the following detailed description, numerous specific details and embodiments are set forth in order to provide a thorough understanding of the anti-viral, anti-bacterial, and anti-tumoral chain terminator compounds disclosed herein, including descriptions of the synthetic 3, 4-didehydro 3′-(“ddH-” or “ddh-”) or deoxy-3, 4-didehydro (“deoxy-ddH-” or “deoxy-ddh-”) compounds, and methods of use thereof for terminating polynucleotide chain synthesis in a cell; methods of use thereof for treating a disease; and methods for producing these compounds. In some instances, well-known methods, procedures, and components have not been described in detail so as not to obscure the present disclosure.

In some embodiments, the synthetic ddh- and deoxy-ddh-compounds represented by the structure of Formula A

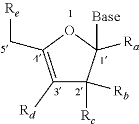

or a pharmaceutically acceptable salt thereof, is synthesized chemically.

In some embodiments, a ddh- and deoxy-ddh-compound or a pharmaceutically acceptable salt thereof, described herein, comprises substituted compounds wherein the overall structure of the nucleotide/nucleosides represented by a structure of Formula A may include: (1) attached to C1′ of the 5 membered ring (to the ddh- or deoxy-ddh-ribose sugar analog); (a) an N-linked natural base; or (b) an N-linked modified purine or pyrimidine base; or (c) a C-linked base and/or (2) different substitutions on the 5 member ring at positions C1′, C2′, C3′, and/or C5′). In some embodiments, a synthetic ddh- and deoxy-ddh-compound or a pharmaceutically acceptable salt thereof, comprises a C-linked nucleoside. In some embodiments, a synthetic ddh- and deoxy-ddh-compound or a pharmaceutically acceptable salt thereof, comprises a N-linked nucleoside. In some embodiments, a synthetic ddh- and deoxy-ddh-compound or a pharmaceutically acceptable salt thereof, comprises a C-linked nucleotide. In some embodiments, a synthetic ddh- and deoxy-ddh-compound or a pharmaceutically acceptable salt thereof, comprises a N-linked nucleotide.

In some embodiments, the ddh- and deoxy-ddh-compounds or a pharmaceutically acceptable salt thereof disclosed herein, comprise unique novel activities. Accordingly, in some embodiments, these ddh- and deoxy-ddh-compounds or a pharmaceutically acceptable salt thereof, add unique therapeutic compounds for methods of use, for example but not limited to treating viral or bacterial infections, or cancer in a subject.

One skilled in the art would appreciate that the term “analog” may encompass a molecule having a structure similar to that of another molecule, but differing from it in respect to a certain component. In some embodiments, the terms “analog”, “structural analog”, “chemical analog” and “substrate analog” are used herein interchangeably, having all the same qualities and meanings.

A skilled artisan would appreciate that the ddh-analog compounds and deoxy-ddh-analog compounds described in detail herein, encompass compounds as described herein below, including compounds of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16.

In some embodiments, a ddh- and deoxy-ddh-compound disclosed herein comprises a structure of a nucleoside or nucleotide analog comprising substituted compounds wherein the ddh- and deoxy-ddh-compounds mimic the overall structure of nucleotide or nucleoside analogs and may include: (1) an N-linked natural base; (2) an N-linked modified purine or pyrimidine base; or (3) a C-linked base. In some embodiments, the ddh- and deoxy-ddh-compounds comprising (1) an N-linked natural base; (2) an N-linked modified purine or pyrimidine base; or (3) a C-linked base, comprise a pharmaceutically acceptable salt of the compound. These compounds mimic the overall structure of nucleotide and nucleoside analogs, and in some embodiments, comprise unique novel activities.

Accordingly, these synthetic ddh- and deoxy-ddh-compounds may add unique therapeutic compounds to the medicinal chemistry arsenal for the treatment of viral infection, diseases associated with viral infections including cancer, and cancer. In some embodiments, use of these substituted ddh- or deoxy-ddh-analog compounds comprises a combination therapy wherein an additional therapeutic agent is included. In some embodiments, the additional therapeutic agent comprises an anti-viral agent, an anti-bacterial agent, or an anti-cancer agent.

In some embodiments, the terms “ddh- and deoxy-ddh-analog compounds”, “ddh- and deoxy-ddh-compounds”, “ddh-analog compounds”, “deoxy-ddh-analog compounds”, “ddh- and deoxy-ddh-prodrugs”, “ddh-prodrugs”, “deoxy-ddh-prodrugs”, “ddh- and deoxy-ddh-analog prodrug compounds”, “analog compounds”, or “compounds”, and grammatical variants thereof, may be used interchangeably herein having all the same meanings and qualities, wherein a “ddh- and deoxy-ddh-analog compound” encompasses a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16. The term “deoxy-ddh-”, “ddh-d-”, and “ddh-deoxy” may in some embodiments be used herein interchangeably, having all the same qualities and meanings.

In some embodiments, ddh- and deoxy-ddh-analog compounds disclosed herein comprise a nucleotide or nucleoside analog comprising a 3′,4′-didehydro or a 3′-deoxy-3′,4′-didehydro modification of the ribose, respectively. In some embodiments, ddh- and deoxy-ddh-analog compounds disclosed herein comprise a nucleotide or nucleoside analog comprising a 3′,4′-didehydro or a 3′-deoxy-3′,4′-didehydro modification of the ribose, respectively, and further comprise a 2′-hydroxy or 2′-deoxy or any other modification of the ribose in addition to the 3′,4′-didehydro or 3′-deoxy-3′,4′-didehydro modifications. In some embodiments, ddh- and deoxy-ddh-analog-compounds comprise compounds wherein the 5-member ring (ribose sugar or analog) may include: (1) different substitutions on the 5 member ring (position 1′, 2′, 3′ and/or 4′); (2) a protective group or/and phosphate moiety or hydroxy linked to position 5′ at position 5′; or (3) natural or unnatural bases C—, or N—, linked to the 5 membered-ring at position 1′; or any combinations thereof.

In some embodiments, ddh- and deoxy-ddh-compounds of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 may be used as DNA or RNA chain terminators.

In some embodiments, while a molecule in the nucleotide form can be a DNA or RNA chain terminator, its corresponding nucleoside form, without any phosphate group, can cross a cell membrane and enter into a cell. In some embodiments, a cell membrane comprises any of a plasma membrane, a nuclear membrane, an organellar membrane, an ER membrane, a Golgi membrane, or a mitochondrial membrane, or a combination thereof.

Similarly, in some embodiments, ddh- and deoxy-ddh-prodrug compounds represented by the structures of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 can cross a cell membrane and enter a cell. In some embodiments, a cell comprises a eukaryotic cell. In some embodiments, a cell comprises a prokaryotic cell. In some embodiments, a cell comprises a eukaryotic or prokaryotic cell. In some embodiments, a cell membrane comprises any of a plasma membrane, a nuclear membrane, an organellar membrane, an ER membrane, a Golgi membrane, or a mitochondrial membrane, or a combination thereof.

Once inside the cell, the compounds of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 as described herein, may be further phosphorylated by one or more cellular kinases to become a diphosphate or a triphosphate-nucleotide. Each step of phosphorylation can be mediated by the same or different cellular kinases. For example, a compound of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 as described herein, may in some embodiments, be converted to a diphosphate nucleotide by a first kinase and the diphosphate nucleotide is converted to triphosphate nucleotide by yet another kinase.

In some embodiments, the novel ddh- and deoxy-ddh-compounds of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 are chemically synthesized using methods known in the art. In some embodiments, the novel ddh- and deoxy-ddh-compounds are provided in the form of a prodrug. These ddh- and deoxy-ddh-compounds can then be analyzed in vitro and in vivo to see whether they are able to be converted to triphosphate nucleotides, which possess DNA and or RNA chain termination functionality.

In some embodiments, the ddh- and deoxy-ddh-compounds of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, and A16 can be used to block cellular DNA or RNA replication. In some embodiments, the ddh- and deoxy-ddh-compounds can be used to treat a disease in a subject in need thereof. Methods of use of these ddh- and deoxy-ddh-compounds includes treating viral infections, wherein said virus comprises an RNA or a DNA virus. In some embodiments, the virus is an RNA virus. In some embodiments, the virus is a DNA virus. In some embodiments, the virus is a double stranded DNA virus. In some embodiments, the virus is a single stranded DNA ((+) sense DNA) virus. In some embodiments, the virus is a (+) ssRNA virus ((+) sense RNA). In some embodiments, the virus is a (−) ssRNA virus ((−) sense RNA). In some embodiments, the virus is a ssRNA-RT virus ((+) sense RNA with DNA intermediate in life-cycle). In some embodiments, the virus is a dsDNA-RT virus.

In some embodiments, the ddh- and deoxy-ddh-compounds, can be synthesized and administered directly a subject. Upon entering the cells, these ddh- and deoxy-ddh-compounds can be phosphorylated by one or more cellular kinases to produce active forms of the compound that can inhibit DNA and or RNA replication, or both.

In certain embodiments, the ddh- and deoxy-ddh-compounds described herein can be used to block cellular DNA or RNA replication or a combination thereof, or treat a disease in a subject. In some embodiments, the ddh- and deoxy-ddh-compounds are administered to cells in a form that can enter the cells (e.g., monophosphate form; prodrug form). Once inside the cells, these the ddh- and deoxy-ddh-compounds can be converted to the active triphosphate (e.g., phosphorylated by one or more cellular kinases).

### Compounds

In some embodiments, provided herein is a ddh- or a deoxy-ddh-compound represented by the structure of Formula I:

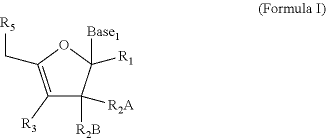

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
R₅ is

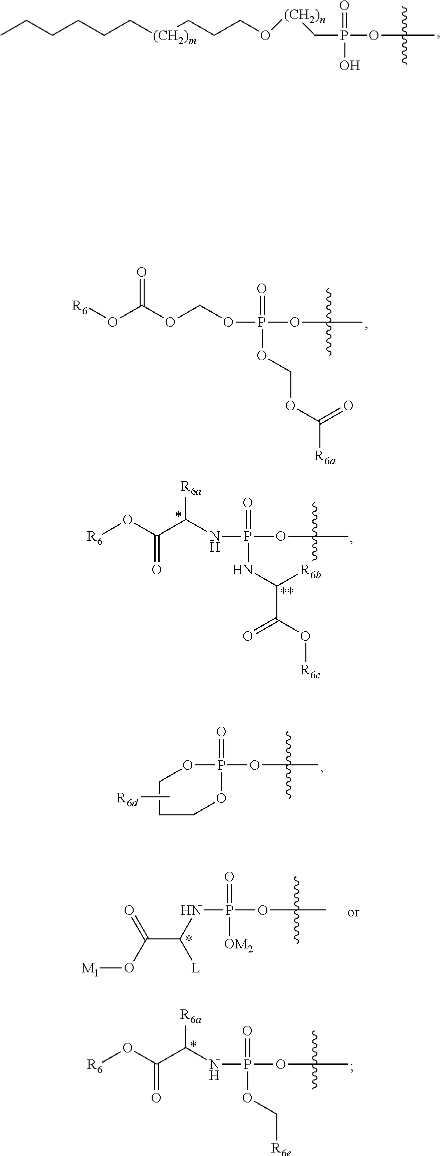

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₁ is an N-linked natural base, wherein the N-linked natural base comprises

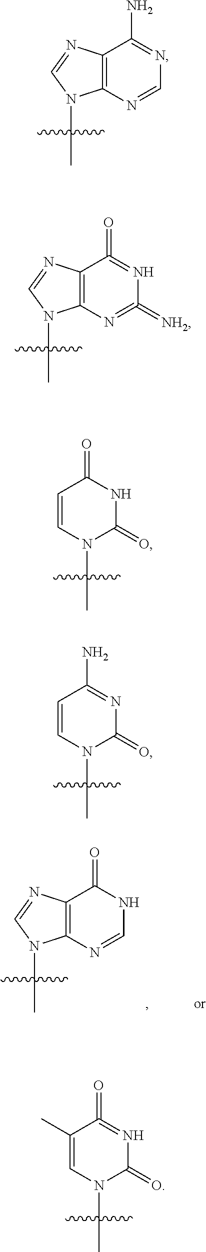

In some embodiments, provided herein is a ddh- or a deoxy-ddh-compound represented by the structure of Formula I

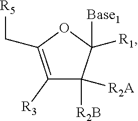

wherein Formula I is represented by Formulas I1-I6 or a pharmaceutically acceptable salt thereof,
wherein
R₁, R₂A, R₂B, R₃, R₅, R₆, R₆ₐ, R_{6b}, R_{6c}, R_{6d}, R₆ₑ, R₈, R₉, R₁₀, n, and m are as defined in Formula I, and Base₁ is a N-linked natural base represented by the following structure in Table 1.

TABLE 1
N-linked natural base ddh-or deoxy ddh-analog compounds

| Formula number | Base₁- N-linked natural base |
|---|---|
| I1 | 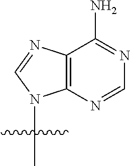 [SMILES: *n1cnc2c(N)ncnc21] |
| I2 | 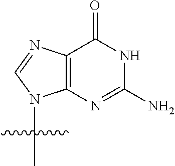 [SMILES: *n1cnc2c(=O)[nH]c(N)nc21] |
| I3 | 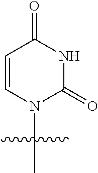 [SMILES: *n1ccc(=O)[nH]c1=O] |
| I4 | 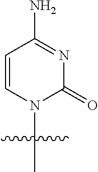 [SMILES: *n1ccc(N)nc1=O] |
| I5 | 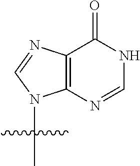 [SMILES: *n1cnc2c(=O)[nH]cnc21] |
| I6 | 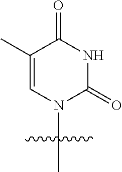 [SMILES: *n1cc(C)c(=O)[nH]c1=O] |

In some embodiments, provided herein is a compound represented by the structure of Formula I1 or a pharmaceutically acceptable salt thereof.

In some embodiments, provided herein is a compound represented by the structure of Formula I2 or a pharmaceutically acceptable salt thereof.

In some embodiments, provided herein is a compound represented by the structure of Formula I3 or a pharmaceutically acceptable salt thereof.

In some embodiments, provided herein is a compound represented by the structure of Formula I4 or a pharmaceutically acceptable salt thereof.

In some embodiments, provided herein is a compound represented by the structure of Formula I5 or a pharmaceutically acceptable salt thereof.

In some embodiments, provided herein is a compound represented by the structure of Formula I6 or a pharmaceutically acceptable salt thereof.

In some embodiments, provided herein is a ddh- or a deoxy-ddh-compound represented by the structure of Formula II

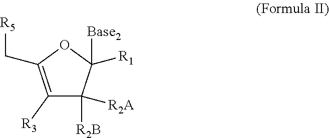

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
R₅ is

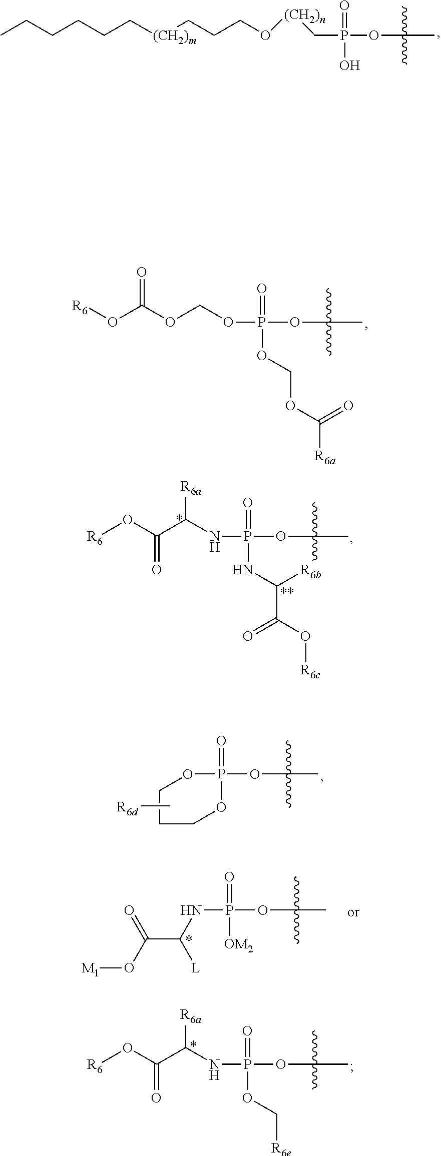

n is an integer between 1-4;
m is an integer between 1-4;
R₆, R₆ₐ, R_{6b}, and R_{6c} are each independently a branched or linear alkyl;
R_{6d}, is H or an alkyl;
R₆ₑ is a haloalkyl;
R₈, R₉, and R₁₀ are each independently an alkyl; and
Base₂ is an N-linked modified purine or pyrimidine base, wherein the N-linked modified purine or pyrimidine base comprises:

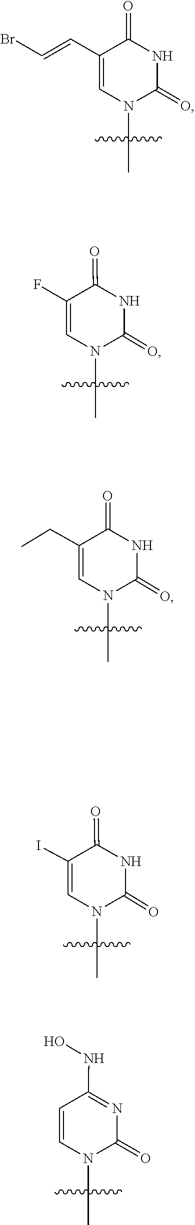 [SMILES: *n1cc(/C=C/Br)c(=O)[nH]c1=O ; *n1cc(F)c(=O)[nH]c1=O ; *n1cc(CC)c(=O)[nH]c1=O ; *n1cc(I)c(=O)[nH]c1=O ; *n1ccc(NO)nc1=O]

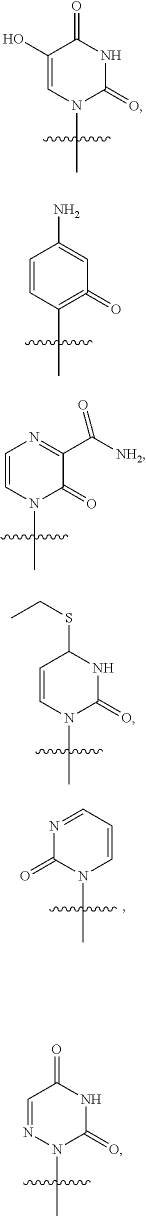

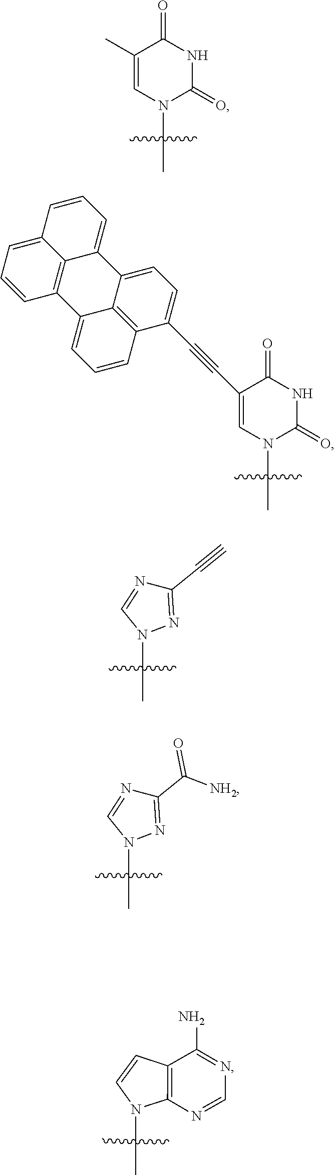

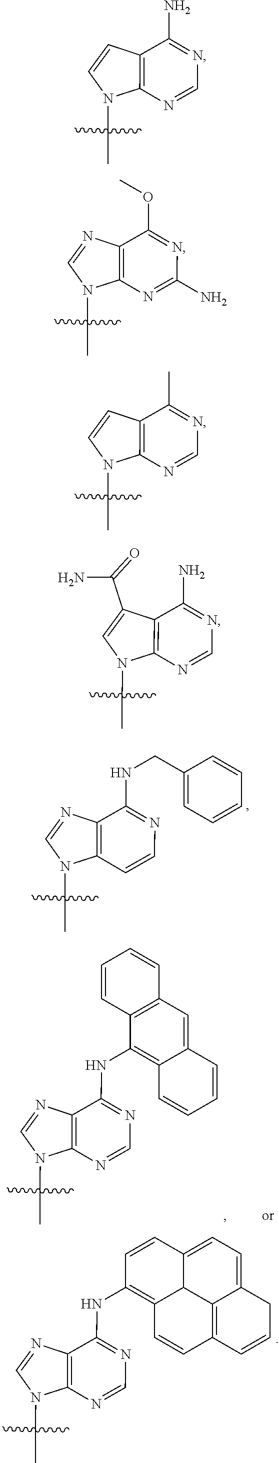

In some embodiments, provided herein is a compound represented by the structure of Formula II

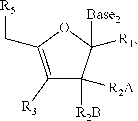

wherein Formula II is represented by Formulas II1-II23 or a pharmaceutically acceptable salt thereof,
wherein
R₁, R₂A, R₂B, R₃, R₅, R₆, R₆ₐ, R_{6b}, R_{6c}, R_{6d}, R₆ₑ, R₈, R₉, R₁₀, n and m are as defined in Formula II, and Base₂ is a N-linked modified purine or pyrimidine base represented by the following structure in Table 2.

TABLE 2
N-linked modified purine or pyrimidine base ddh-or deoxy ddh-analog
compounds

| Formula | |
|---|---|
| number | Base₂- N-linked modified purine or pyrimidine base |
| II1 | 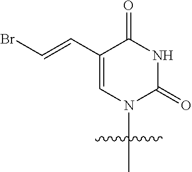 [SMILES: *n1cc(/C=C/Br)c(=O)[nH]c1=O] |
| II2 | 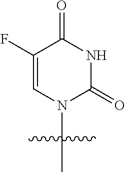 [SMILES: *n1cc(F)c(=O)[nH]c1=O] |
| II3 | 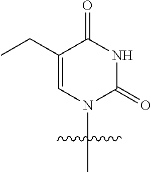 [SMILES: *n1cc(CC)c(=O)[nH]c1=O] |
| II4 | 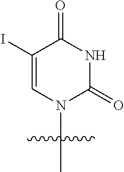 [SMILES: *n1cc(I)c(=O)[nH]c1=O] |
| II5 | 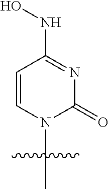 [SMILES: *n1ccc(NO)nc1=O] |
| II6 | 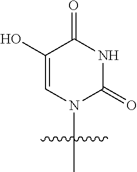 [SMILES: *n1cc(O)c(=O)[nH]c1=O] |
| II7 | 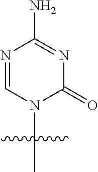 [SMILES: *n1cnc(N)nc1=O] |
| II8 | 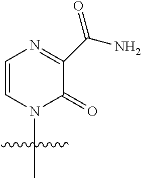 [SMILES: *n1ccnc(C(N)=O)c1=O] |
| II9 | 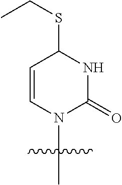 [SMILES: *N1C=CC(SCC)NC1=O] |
| II10 | 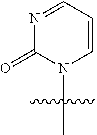 [SMILES: *n1cccnc1=O] |
| II11 | 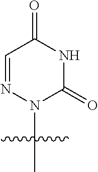 [SMILES: *n1ncc(=O)[nH]c1=O] |
| II12 | 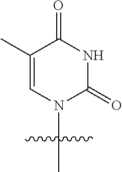 [SMILES: *n1cc(C)c(=O)[nH]c1=O] |
| II13 | 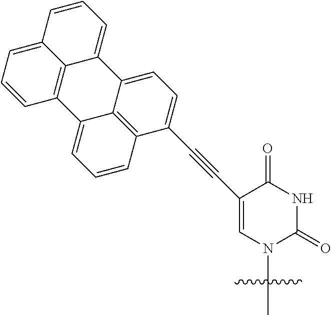 [SMILES: *n1cc(C#Cc2ccc3c4cccc5cccc(c6cccc2c63)c54)c(=O)[nH]c1=O] |
| II14 | 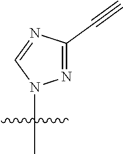 [SMILES: *n1cnc(C#C)n1] |
| II15 | 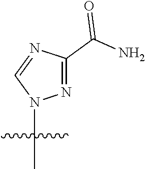 [SMILES: *n1cnc(C(N)=O)n1] |
| II16 | 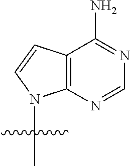 [SMILES: *n1ccc2c(N)ncnc21] |
| II17 | 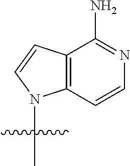 [SMILES: *n1ccc2c(N)nccc21] |
| II18 | 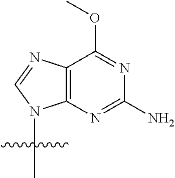 [SMILES: *n1cnc2c(OC)nc(N)nc21] |
| II19 | 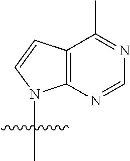 [SMILES: *n1ccc2c(C)ncnc21] |
| II20 | 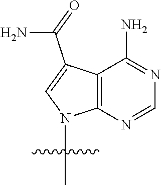 [SMILES: *n1cc(C(N)=O)c2c(N)ncnc21] |
| II21 | 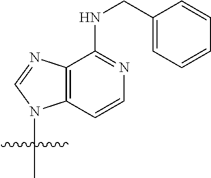 [SMILES: *n1cnc2c(NCc3ccccc3)nccc21] |
| II22 | 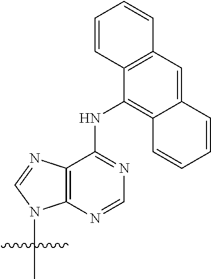 [SMILES: *n1cnc2c(Nc3c4ccccc4cc4ccccc34)ncnc21] |
| II23 | 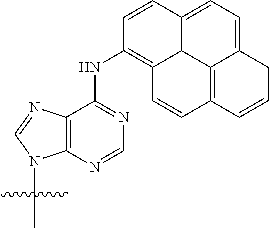 [SMILES: *n1cnc2c(NC3=C4C=CC5=C6C(=CC=C(C=C3)C46)CC=C5)ncnc21] |

In some embodiments, provided herein is a compound represented by the structure of Formula II1 or a pharmaceutically acceptable salt thereof.

In some embodiments, provided herein is a compound represented by the structure of Formula II2 or a pharmaceutically acceptable salt thereof.

In some embodiments, provided herein is a compound represented by the structure of Formula II3 or a pharmaceutically acceptable salt thereof.

In some embodiments, provided herein is a compound represented by the structure of Formula II4 or a pharmaceutically acceptable salt thereof.

In some embodiments, provided herein is a compound represented by the structure of Formula II5 or a pharmaceutically acceptable salt thereof.

In some embodiments, provided herein is a compound represented by the structure of Formula II6 or a pharmaceutically acceptable salt thereof.

In some embodiments, provided herein is a compound represented by the structure of Formula II7 or a pharmaceutically acceptable salt thereof.

In some embodiments, provided herein is a compound represented by the structure of Formula II8 or a pharmaceutically acceptable salt thereof.

In some embodiments, provided herein is a compound represented by the structure of Formula II9 or a pharmaceutically acceptable salt thereof.

In some embodiments, provided herein is a compound represented by the structure of Formula II10 or a pharmaceutically acceptable salt thereof.

In some embodiments, provided herein is a compound represented by the structure of Formula II11 or a pharmaceutically acceptable salt thereof.

In some embodiments, provided herein is a compound represented by the structure of Formula II12 or a pharmaceutically acceptable salt thereof.

In some embodiments, provided herein is a compound represented by the structure of Formula II13 or a pharmaceutically acceptable salt thereof.

In some embodiments, provided herein is a compound represented by the structure of Formula II14 or a pharmaceutically acceptable salt thereof.

In some embodiments, provided herein is a compound represented by the structure of Formula II15 or a pharmaceutically acceptable salt thereof.

In some embodiments, provided herein is a compound represented by the structure of Formula II16 or a pharmaceutically acceptable salt thereof.

In some embodiments, provided herein is a compound represented by the structure of Formula II17 or a pharmaceutically acceptable salt thereof.

In some embodiments, provided herein is a compound represented by the structure of Formula II18 or a pharmaceutically acceptable salt thereof.

In some embodiments, provided herein is a compound represented by the structure of Formula II19 or a pharmaceutically acceptable salt thereof.

In some embodiments, provided herein is a compound represented by the structure of Formula II20 or a pharmaceutically acceptable salt thereof.

In some embodiments, provided herein is a compound represented by the structure of Formula II21 or a pharmaceutically acceptable salt thereof.

In some embodiments, provided herein is a compound represented by the structure of Formula II22 or a pharmaceutically acceptable salt thereof.

In some embodiments, provided herein is a compound represented by the structure of Formula II23 or a pharmaceutically acceptable salt thereof.

In some embodiments, provided herein is a ddh- or a deoxy-ddh-compound represented by the structure of Formula III

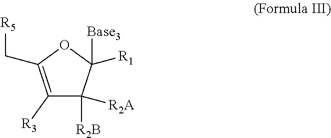

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
R₅ is

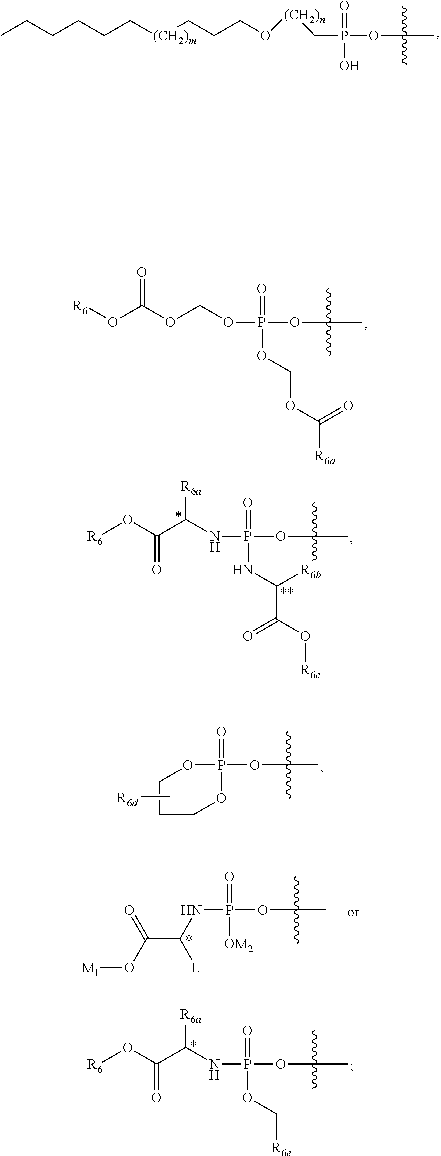

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₃ is a C-linked base, wherein the C-linked base comprises

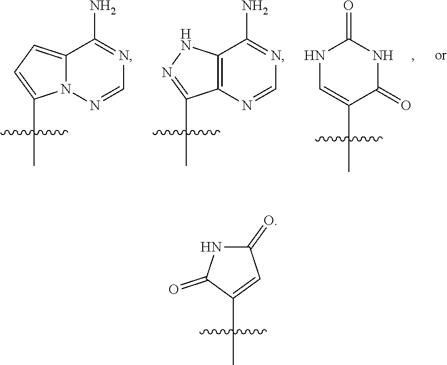

In some embodiments, provided herein is a ddh- or a deoxy-ddh-compound represented by the structure of Formula III

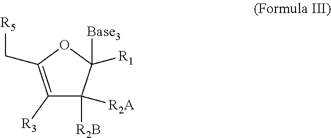

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
R₅ is

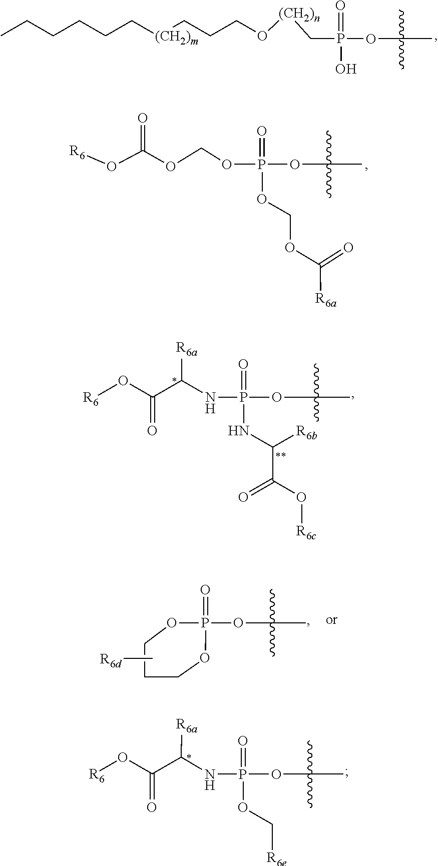

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₃ is a C-linked base, wherein the C-linked base comprises

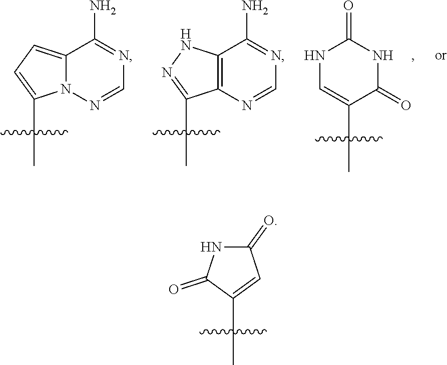

In some embodiments, provided herein is a compound represented by the structure of Formula III,

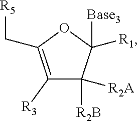

wherein Formula III is represented by Formulas III1-III4 or a pharmaceutically acceptable salt thereof,
wherein
R₁, R₂A, R₂B, R₃, R₅, R₆, R₆ₐ, R_{6b}, R_{6c}, R_{6d}, R₆ₑ, R₈, R₉, R₁₀, n and m are as defined in Formula III, and Base₃ is a C-linked base represented by the following structure in Table 3.

TABLE 3
C-linked base- based ddh-or deoxy ddh-analog compounds

| Formula number | Base₃- C-linked base |
|---|---|
| III1 | 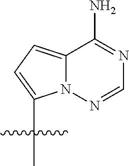 [SMILES: *c1ccc2c(N)ncnn12] |
| III2 | 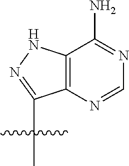 [SMILES: *c1n[nH]c2c(N)ncnc12] |
| III3 | 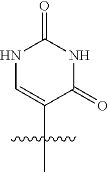 [SMILES: *c1c[nH]c(=O)[nH]c1=O] |
| III4 | 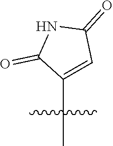 [SMILES: *C1=CC(=O)NC1=O] |

In some embodiments, provided herein is a compound represented by the structure of Formula III1 or a pharmaceutically acceptable salt thereof.

In some embodiments, provided herein is a compound represented by the structure of Formula III2 or a pharmaceutically acceptable salt thereof.

In some embodiments, provided herein is a compound represented by the structure of Formula III3 or a pharmaceutically acceptable salt thereof.

In some embodiments, provided herein is a compound represented by the structure of Formula III4 or a pharmaceutically acceptable salt thereof.

In some embodiments, provided herein is a ddh- or a deoxy-ddh-compound represented by the structure of Formula IV

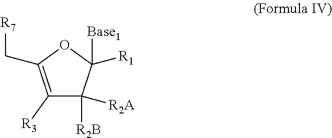

or a pharmaceutically acceptable salt thereof,
wherein
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is halo, H or an alkyl;
wherein:
when R₃ is a halo, then R₁ is H, —CN, —N₃, an alkyne-R₈, or an alkyl; when R₃ is H or an alkyl, then R₁ is —N₃, an alkyne-R₈, or an alkyl;
R₇ is OH

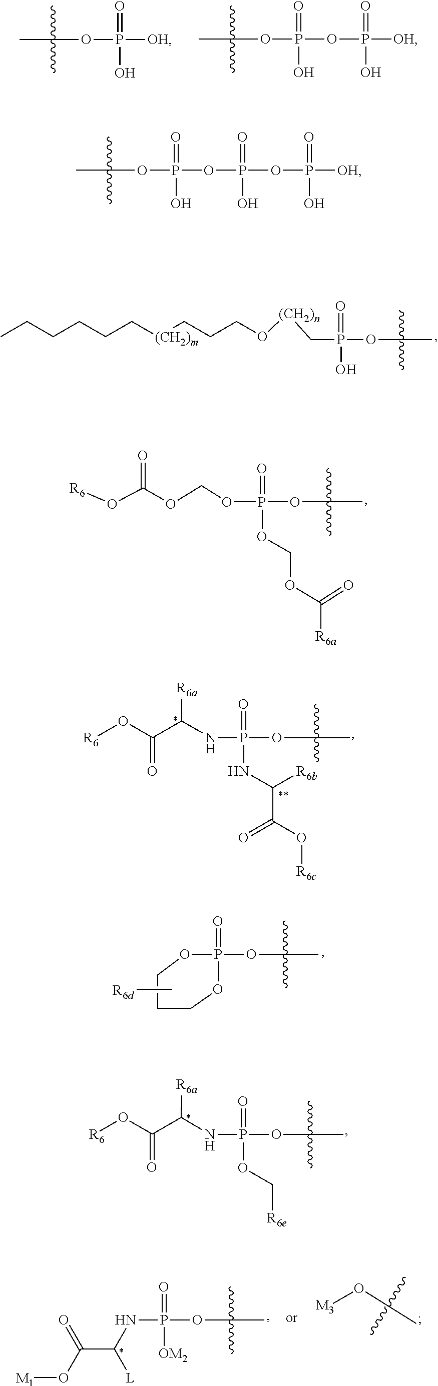

M₁ is a branched or linear alkyl;
L is a side chain of a natural or unnatural amino acid;
M₂ is substituted or unsubstituted aryl, or substituted or unsubstituted heteroaryl;
M₃ is

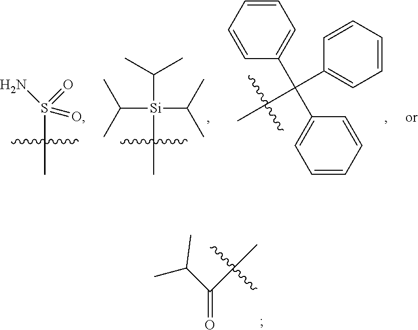

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₁ is a N-linked natural base, wherein the N-linked natural base comprises:

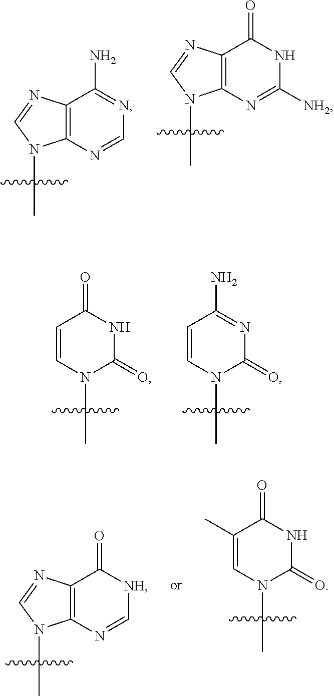

In some embodiments, provided herein is a ddh- or a deoxy-ddh-compound represented by the structure of Formula IV

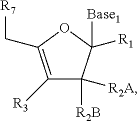

wherein Formula IV is represented by Formulas IV1-IV6 or a pharmaceutically acceptable salt thereof,
wherein
R₁, R₂A, R₂B, R₃, R₆, R₆ₐ, R_{6b}, R_{6c}, R_{6d}, R₆ₑ, R₇, R₈, R₉, R₁₀, n, m, M₁, M₂, M₃ and L are as defined in Formula IV, and Base₁ is a N-linked natural base represented by the following structure in Table 4.

TABLE 4
N-linked natural base ddh-or deoxy ddh-analog compounds

| Formula number | Base₁- N-linked natural base |
|---|---|
| IV1 | 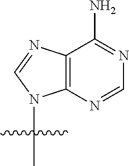 [SMILES: *n1cnc2c(N)ncnc21] |
| IV2 | 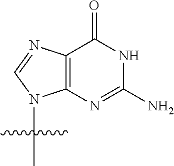 [SMILES: *n1cnc2c(=O)[nH]c(N)nc21] |
| IV3 | 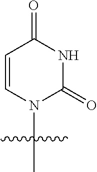 [SMILES: *n1ccc(=O)[nH]c1=O] |
| IV4 | 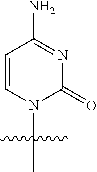 [SMILES: *n1ccc(N)nc1=O] |
| IV5 | 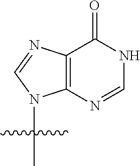 [SMILES: *n1cnc2c(=O)[nH]cnc21] |
| IV6 | 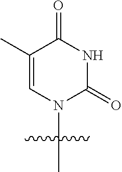 [SMILES: *n1cc(C)c(=O)[nH]c1=O] |

In some embodiments, provided herein is a compound represented by the structure of Formula IV1 or a pharmaceutically acceptable salt thereof.

In some embodiments, provided herein is a compound represented by the structure of Formula IV2 or a pharmaceutically acceptable salt thereof.

In some embodiments, provided herein is a compound represented by the structure of Formula IV3 or a pharmaceutically acceptable salt thereof.

In some embodiments, provided herein is a compound represented by the structure of Formula IV4 or a pharmaceutically acceptable salt thereof.

In some embodiments, provided herein is a compound represented by the structure of Formula IV5 or a pharmaceutically acceptable salt thereof.

In some embodiments, provided herein is a compound represented by the structure of Formula IV6 or a pharmaceutically acceptable salt thereof.

In some embodiments, provided herein is a ddh- or a deoxy-ddh-compound represented by the structure of Formula V

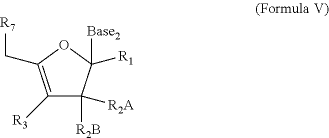

or a pharmaceutically acceptable salt thereof,
wherein
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is halo, H or an alkyl;
wherein:
when R₃ is a halo, then R₁ is H, —CN, —N₃, an alkyne-R₈, or an alkyl;
when R₃ is H or an alkyl, then R₁ is —N₃, an alkyne-R₈, or an alkyl;
R₇ is OH,

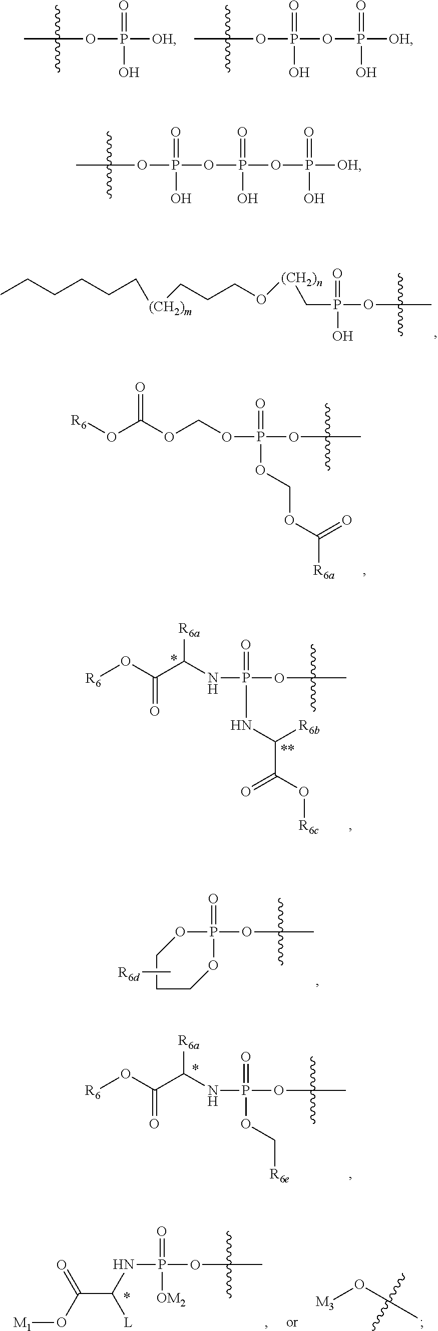

M₁ is a branched or linear alkyl;
L is a side chain of a natural or unnatural amino acid;
M₂ is substituted or unsubstituted aryl, or substituted or unsubstituted heteroaryl;
M₃ is

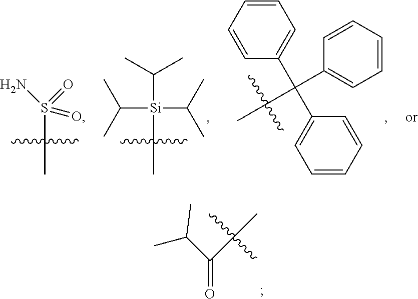

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₂ is a N-linked modified purine or pyrimidine base, wherein the N-linked modified purine or pyrimidine base comprises:

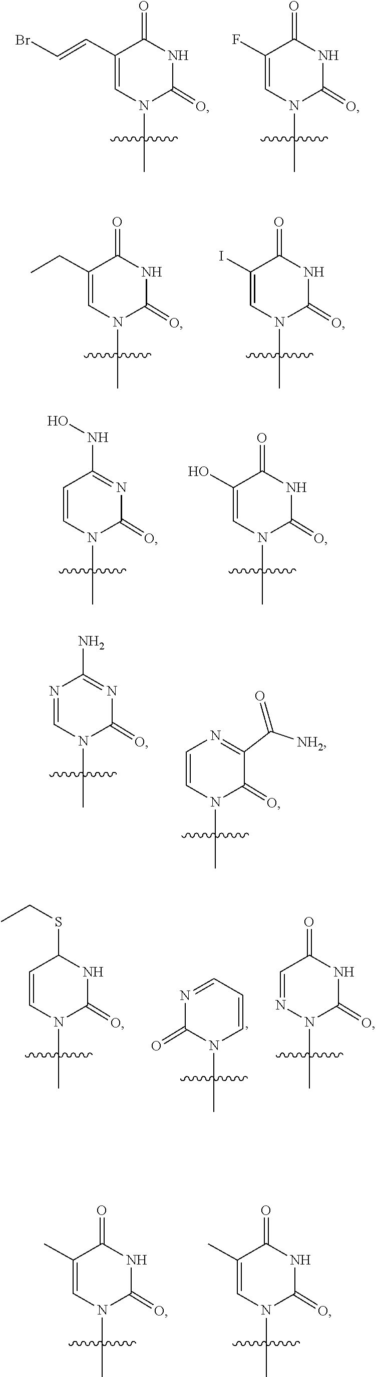

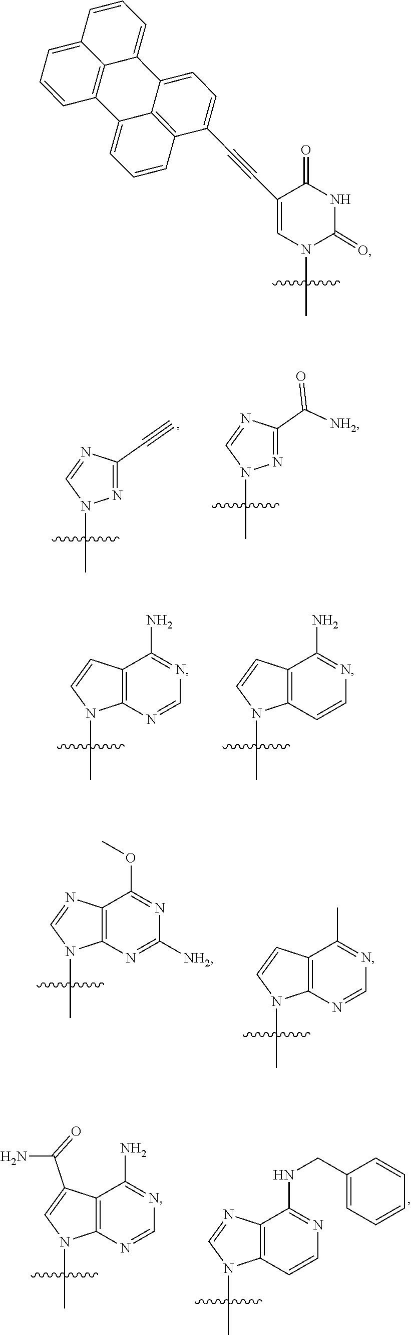

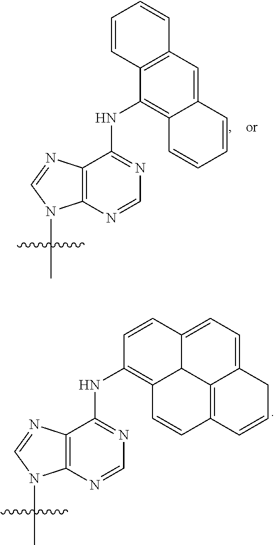 [SMILES: *n1cnc2c(Nc3c4ccccc4cc4ccccc34)ncnc21 ; *n1cnc2c(NC3=C4C=CC5=C6C(=CC=C(C=C3)C46)CC=C5)ncnc21]

In some embodiments, provided herein is a compound represented by the structure of Formula V

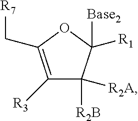

wherein Formula V is represented by Formulas V1-V23 or a pharmaceutically acceptable salt thereof,
wherein
R₁, R₂A, R₂B, R₃, R₆, R₆ₐ, R_{6b}, R_{6c}, R_{6d}, R₆ₑ, R₇, R₈, R₉, R₁₀, n, m, M₁, M₂, M₃ and L are as defined in Formula V, and Base₂ is a N-linked modified purine or pyrimidine base represented by the following structure in Table 5.

TABLE 5
N-linked modified purine or pyrimidine base ddh-or deoxy ddh-
analog compounds.

| Formula | |
|---|---|
| number | Base₂- N-linked modified purine or pyrimidine base |
| V1 | 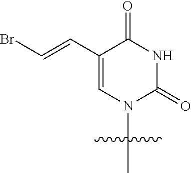 [SMILES: *n1cc(/C=C/Br)c(=O)[nH]c1=O] |
| V2 | 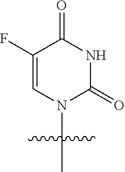 [SMILES: *n1cc(F)c(=O)[nH]c1=O] |
| V3 | 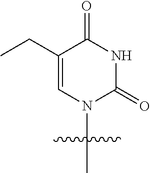 [SMILES: *n1cc(CC)c(=O)[nH]c1=O] |
| V4 | 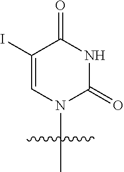 [SMILES: *n1cc(I)c(=O)[nH]c1=O] |
| V5 | 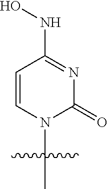 [SMILES: *n1ccc(NO)nc1=O] |
| V6 | 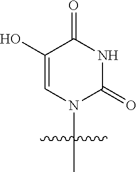 [SMILES: *n1cc(O)c(=O)[nH]c1=O] |
| V7 | 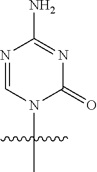 [SMILES: *n1cnc(N)nc1=O] |
| V8 | 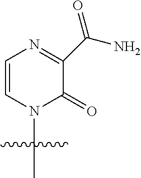 [SMILES: *n1ccnc(C(N)=O)c1=O] |
| V9 | 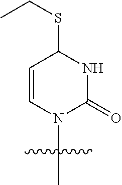 [SMILES: *N1C=CC(SCC)NC1=O] |
| V10 | 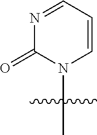 [SMILES: *n1cccnc1=O] |
| V11 | 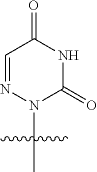 [SMILES: *n1ncc(=O)[nH]c1=O] |
| V12 | 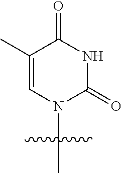 [SMILES: *n1cc(C)c(=O)[nH]c1=O] |
| V13 | 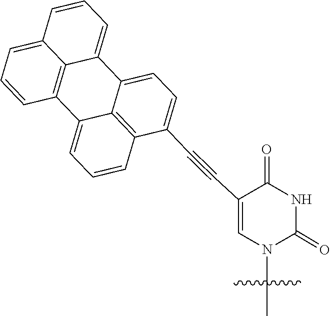 [SMILES: *n1cc(C#Cc2ccc3c4cccc5cccc(c6cccc2c63)c54)c(=O)[nH]c1=O] |
| V14 | 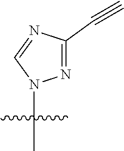 [SMILES: *n1cnc(C#C)n1] |
| V15 | 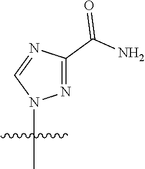 [SMILES: *n1cnc(C(N)=O)n1] |
| V16 | 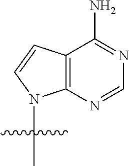 [SMILES: *n1ccc2c(N)ncnc21] |
| V17 | 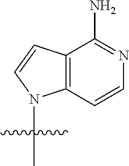 [SMILES: *n1ccc2c(N)nccc21] |
| V18 | 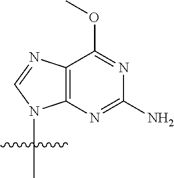 [SMILES: *n1cnc2c(OC)nc(N)nc21] |
| V19 | 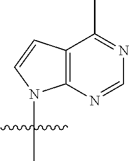 [SMILES: *n1ccc2c(C)ncnc21] |
| V20 | 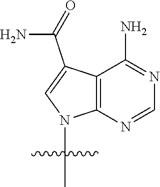 [SMILES: *n1cc(C(N)=O)c2c(N)ncnc21] |
| V21 | 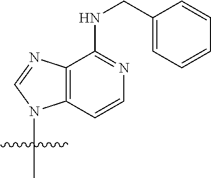 [SMILES: *n1cnc2c(NCc3ccccc3)nccc21] |
| V22 | 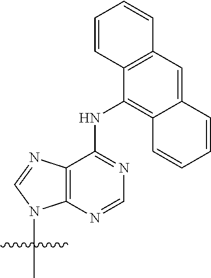 [SMILES: *n1cnc2c(Nc3c4ccccc4cc4ccccc34)ncnc21] |
| V23 | 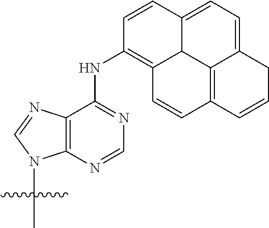 [SMILES: *n1cnc2c(NC3=C4C=CC5=C6C(=CC=C(C=C3)C46)CC=C5)ncnc21] |

In some embodiments, provided herein is a compound represented by the structure of Formula V1 or a pharmaceutically acceptable salt thereof.

In some embodiments, provided herein is a compound represented by the structure of Formula V2 or a pharmaceutically acceptable salt thereof.

In some embodiments, provided herein is a compound represented by the structure of Formula V3 or a pharmaceutically acceptable salt thereof.

In some embodiments, provided herein is a compound represented by the structure of Formula V4 or a pharmaceutically acceptable salt thereof.

In some embodiments, provided herein is a compound represented by the structure of Formula V5 or a pharmaceutically acceptable salt thereof.

In some embodiments, provided herein is a compound represented by the structure of Formula V6 or a pharmaceutically acceptable salt thereof.

In some embodiments, provided herein is a compound represented by the structure of Formula V7 or a pharmaceutically acceptable salt thereof.

In some embodiments, provided herein is a compound represented by the structure of Formula V8 or a pharmaceutically acceptable salt thereof.

In some embodiments, provided herein is a compound represented by the structure of Formula V9 or a pharmaceutically acceptable salt thereof.

In some embodiments, provided herein is a compound represented by the structure of Formula V10 or a pharmaceutically acceptable salt thereof.

In some embodiments, provided herein is a compound represented by the structure of Formula V11 or a pharmaceutically acceptable salt thereof.

In some embodiments, provided herein is a compound represented by the structure of Formula V12 or a pharmaceutically acceptable salt thereof.

In some embodiments, provided herein is a compound represented by the structure of Formula V13 or a pharmaceutically acceptable salt thereof.

In some embodiments, provided herein is a compound represented by the structure of Formula V14 or a pharmaceutically acceptable salt thereof.

In some embodiments, provided herein is a compound represented by the structure of Formula V15 or a pharmaceutically acceptable salt thereof.

In some embodiments, provided herein is a compound represented by the structure of Formula V16 or a pharmaceutically acceptable salt thereof.

In some embodiments, provided herein is a compound represented by the structure of Formula V17 or a pharmaceutically acceptable salt thereof.

In some embodiments, provided herein is a compound represented by the structure of Formula V18 or a pharmaceutically acceptable salt thereof.

In some embodiments, provided herein is a compound represented by the structure of Formula V19 or a pharmaceutically acceptable salt thereof.

In some embodiments, provided herein is a compound represented by the structure of Formula V20 or a pharmaceutically acceptable salt thereof.

In some embodiments, provided herein is a compound represented by the structure of Formula V21 or a pharmaceutically acceptable salt thereof.

In some embodiments, provided herein is a compound represented by the structure of Formula V22 or a pharmaceutically acceptable salt thereof.

In some embodiments, provided herein is a compound represented by the structure of Formula V23 or a pharmaceutically acceptable salt thereof.

In some embodiments, provided herein is a ddh- or a deoxy-ddh-compound represented by the structure of Formula VI

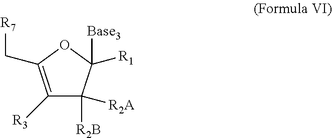

or a pharmaceutically acceptable salt thereof,
wherein
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is halo, H or an alkyl;
wherein:
when R₃ is a halo, then R₁ is H, —CN, —N₃, an alkyne-R₈, or an alkyl;
when R₃ is H or an alkyl, then R₁ is —N₃, an alkyne-R₈, or an alkyl;
R₇ is OH,

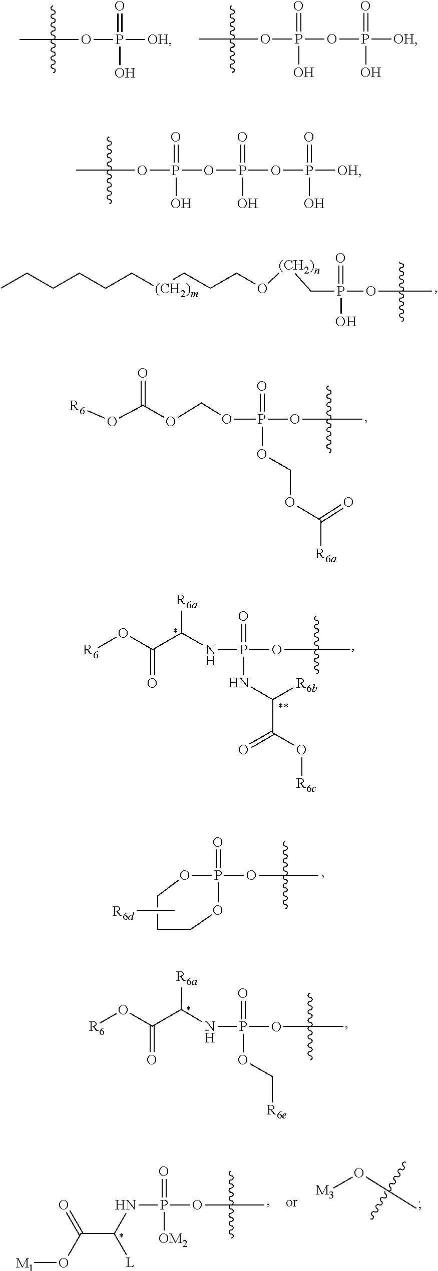

M₁ is a branched or linear alkyl;
L is a side chain of a natural or unnatural amino acid;
M₂ is substituted or unsubstituted aryl, or substituted or unsubstituted heteroaryl;
M₃ is

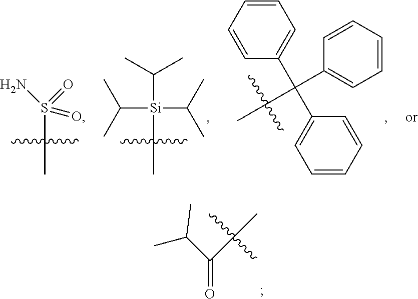

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₃ is a C-linked base, wherein the C-linked base comprises:

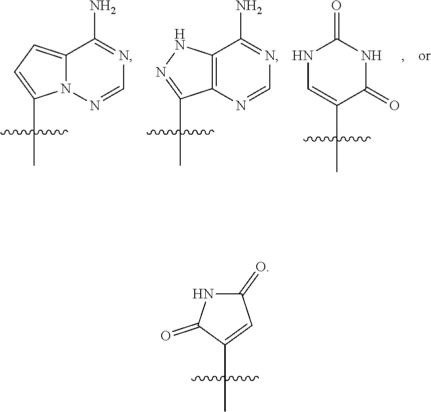

In some embodiments, provided herein is a compound represented by the structure of Formula VI

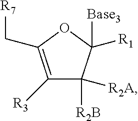

wherein Formula VI is represented by Formulas VI1-VI4 or a pharmaceutically acceptable salt thereof,
wherein
R₁, R₂A, R₂B, R₃, R₆, R₆ₐ, R_{6b}, R_{6c}, R_{6d}, R₆ₑ, R₇, R₈, R₉, R₁₀, n, m, M₁, M₂, M₃ and L are as defined in Formula VI, and Base₃ is a C-linked base represented by the following structure in Table 6.

TABLE 6
C-linked base- based ddh-or deoxy ddh-analog compounds

| Formula number | Base₃- C-linked base |
|---|---|
| VI1 | 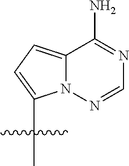 [SMILES: *c1ccc2c(N)ncnn12] |
| VI2 | 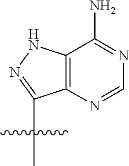 [SMILES: *c1n[nH]c2c(N)ncnc12] |
| VI3 | 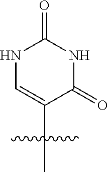 [SMILES: *c1c[nH]c(=O)[nH]c1=O] |
| VI4 | 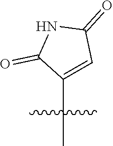 [SMILES: *C1=CC(=O)NC1=O] |

In some embodiments, provided herein is a compound represented by the structure of Formula VI1 or a pharmaceutically acceptable salt thereof.

In some embodiments, provided herein is a compound represented by the structure of Formula VI2 or a pharmaceutically acceptable salt thereof.

In some embodiments, provided herein is a compound represented by the structure of Formula VI3 or a pharmaceutically acceptable salt thereof.

In some embodiments, provided herein is a compound represented by the structure of Formula VI4 or a pharmaceutically acceptable salt thereof.

In some embodiments, the ddh- or a deoxy-ddh-compound of a compound of Formula III or Formula VI is represented by the structures of Compound 15 and Compound 16:

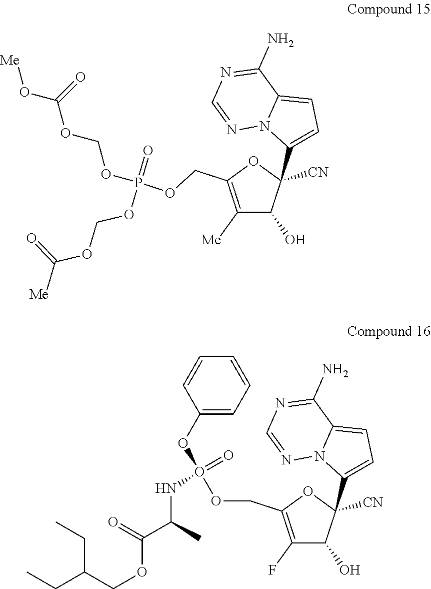

In some embodiments, provided herein is a ddh- or a deoxy-ddh-compound represented by the structure of Formula VII

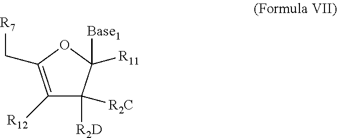

or a pharmaceutically acceptable salt thereof,
wherein
R₁₁ is H or —CN;
R₂C is an alkyl or an alkyne-R₉;
R₂D is an alkyl or an alkyne-R₁₀;
R₁₂ is H or an alkyl;
R₇ is OH,

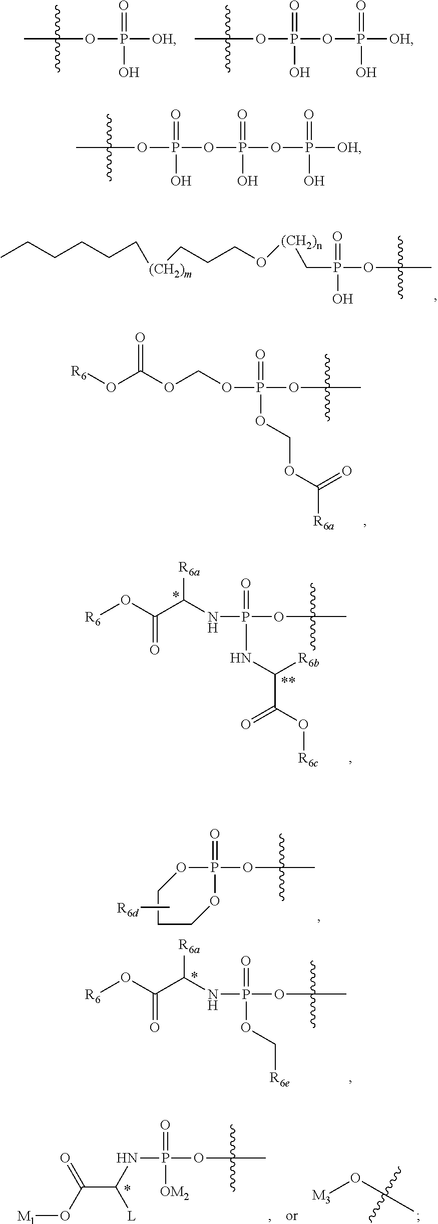

M₁ is a branched or linear alkyl;
L is a side chain of a natural or unnatural amino acid;
M₂ is substituted or unsubstituted aryl, or substituted or unsubstituted heteroaryl;
M₃ is

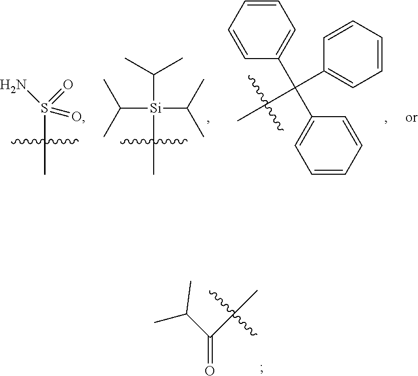

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₉, and R₁₀ are each independently an alkyl; and
Base₁ is a N-linked natural base, wherein the N-linked natural base comprises:

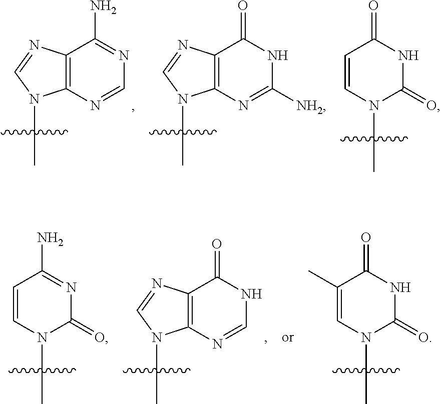

In some embodiments, provided herein is a ddh- or a deoxy-ddh-compound represented by the structure of Formula VII

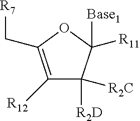

wherein Formula VII is represented by Formulas VII1-VII6 (Formula VII1-VII6) or a pharmaceutically acceptable salt thereof,
wherein
R₁₁, R₂C, R₂D, R₁₂, R₆, R₆ₐ, R_{6b}, R_{6c}, R_{6d}, R₆ₑ, R₇, R₉, R₁₀, n, m, M₁, M₂, M₃ and L are as defined in Formula VII, and Base₁ is a N-linked natural base represented by the following structure in Table 7.

TABLE 7
N-linked natural base ddh-or deoxy ddh-analog compounds

| Formula number | Base₁- N-linked natural base |
|---|---|
| VII1 | 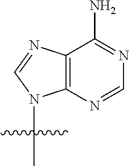 [SMILES: *n1cnc2c(N)ncnc21] |
| VII2 | 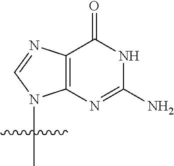 [SMILES: *n1cnc2c(=O)[nH]c(N)nc21] |
| VII3 | 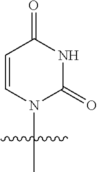 [SMILES: *n1ccc(=O)[nH]c1=O] |
| VII4 | 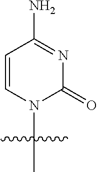 [SMILES: *n1ccc(N)nc1=O] |
| VII5 | 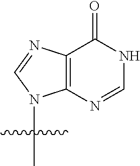 [SMILES: *n1cnc2c(=O)[nH]cnc21] |
| VII6 | 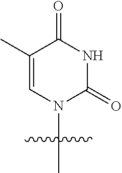 [SMILES: *n1cc(C)c(=O)[nH]c1=O] |

In some embodiments, provided herein is a compound represented by the structure of Formula VII1 or a pharmaceutically acceptable salt thereof.

In some embodiments, provided herein is a compound represented by the structure of Formula VII2 or a pharmaceutically acceptable salt thereof.

In some embodiments, provided herein is a compound represented by the structure of Formula VII3 or a pharmaceutically acceptable salt thereof.

In some embodiments, provided herein is a compound represented by the structure of Formula VII4 or a pharmaceutically acceptable salt thereof.

In some embodiments, provided herein is a compound represented by the structure of Formula VII5 or a pharmaceutically acceptable salt thereof.

In some embodiments, provided herein is a compound represented by the structure of Formula VII6 or a pharmaceutically acceptable salt thereof.

In some embodiments, provided herein is a ddh- or a deoxy-ddh-compound represented by the structure of Formula VIII

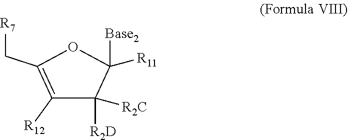

or a pharmaceutically acceptable salt thereof,
wherein
R₁₁ is H, or —CN;
R₂C is an alkyl, or an alkyne-R₉;
R₂D is an alkyl, or an alkyne-R₁₀;
R₁₂ is H or an alkyl;
R₇ is OH,

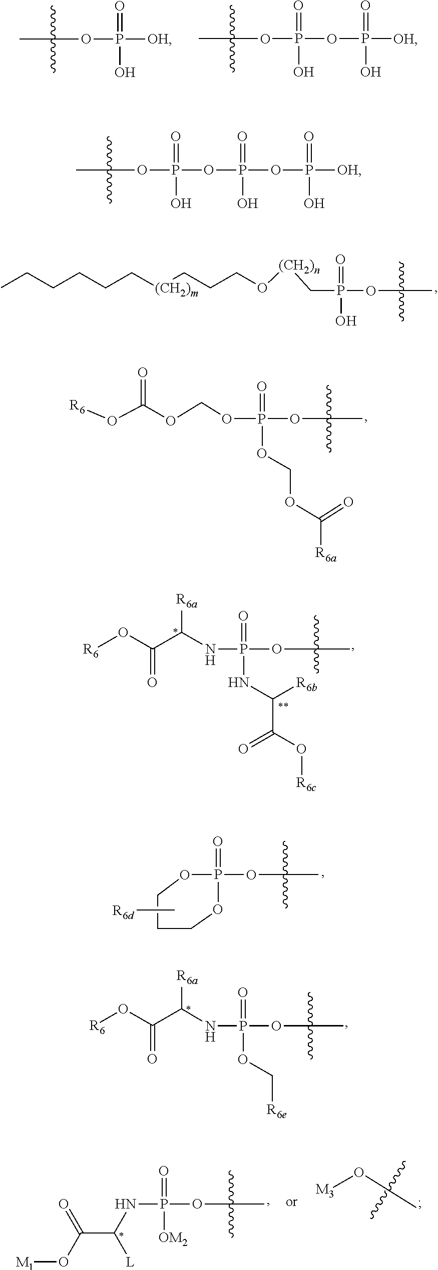

M₁ is a branched or linear alkyl;
L is a side chain of a natural or unnatural amino acid;
M₂ is substituted or unsubstituted aryl, or substituted or unsubstituted heteroaryl;
M₃ is

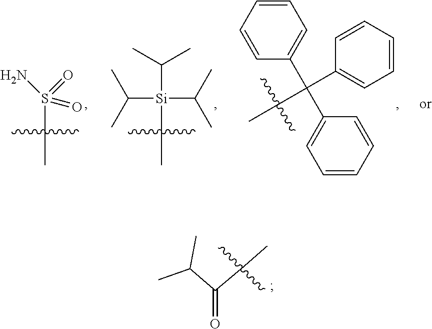

n is an integer between 1-4;
m is an integer between 1-4;
R₆, R₆ₐ, R_{6b}, and R_{6c} are each independently branched or linear alkyl;
R_{6d} is H or an alkyl;
R₆ₑ is a haloalkyl;
R₉, and R₁₀ are each independently an alkyl; and
Base₂ is a N-linked modified purine or pyrimidine base, wherein the N-linked modified purine or pyrimidine base comprises:

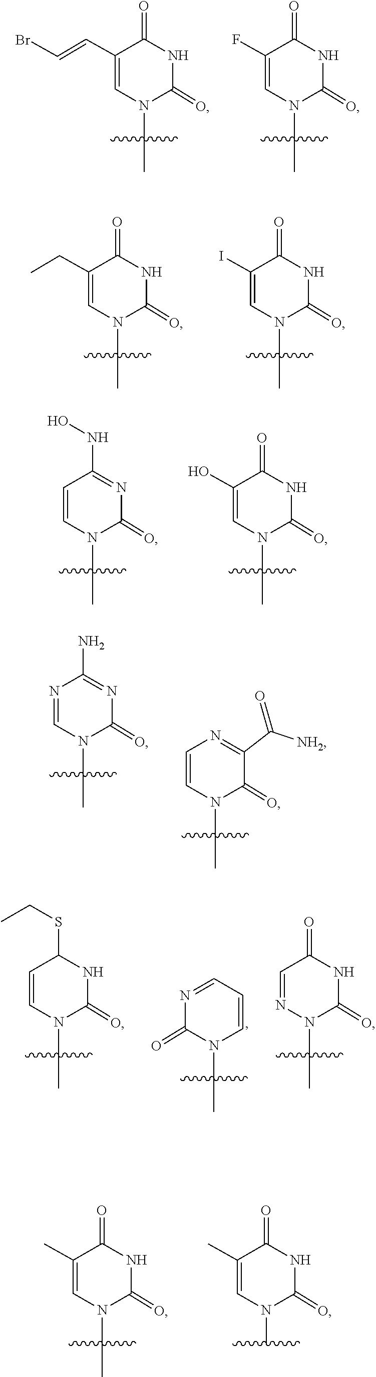

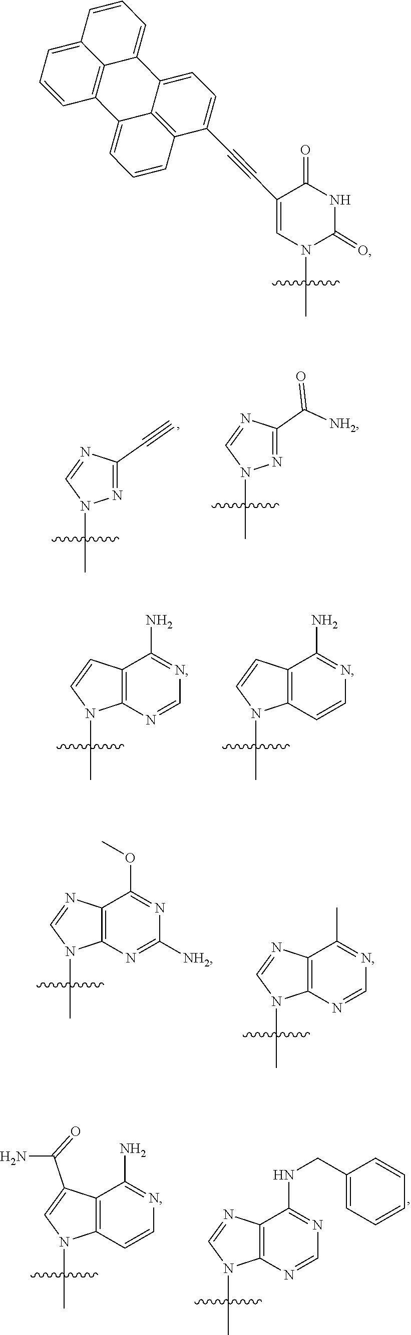

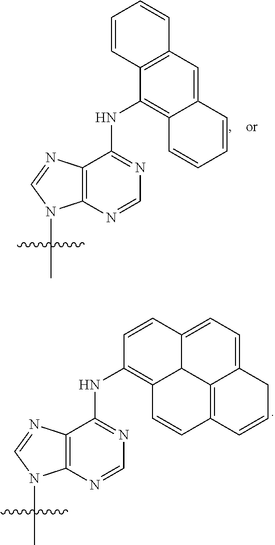 [SMILES: *n1cnc2c(Nc3c4ccccc4cc4ccccc34)ncnc21 ; *n1cnc2c(NC3=C4C=CC5=C6C(=CC=C(C=C3)C46)CC=C5)ncnc21]

In some embodiments, provided herein is a compound represented by the structure of Formula VIII

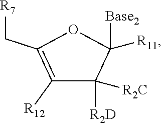

wherein Formula VIII is represented by Formulas VIII1-VIII23 or a pharmaceutically acceptable salt thereof,
wherein R₁₁, R₂C, R₂D, R₁₂, R₆, R₆ₐ, R_{6b}, R_{6c}, R_{6d}, R₆ₑ, R₇, R₉, R₁₀, n, m, M₁, M₂, M₃ and L are as defined in Formula VIII, and Base₂ is a N-linked modified purine or pyrimidine base represented by the following structure in Table 8.

TABLE 8
N-linked modified purine or pyrimidine base ddh-or deoxy ddh-
analog compounds.

| Formula | |
|---|---|
| number | Base₂- N-linked modified purine or pyrimidine base |
| VIII1 | 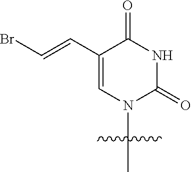 [SMILES: *n1cc(/C=C/Br)c(=O)[nH]c1=O] |
| VIII2 | 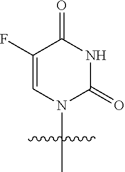 [SMILES: *n1cc(F)c(=O)[nH]c1=O] |
| VIII3 | 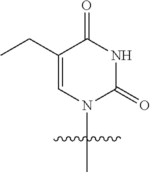 [SMILES: *n1cc(CC)c(=O)[nH]c1=O] |
| VIII4 | 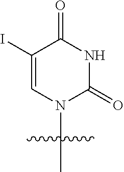 [SMILES: *n1cc(I)c(=O)[nH]c1=O] |
| VIII5 | 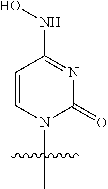 [SMILES: *n1ccc(NO)nc1=O] |
| VIII6 | 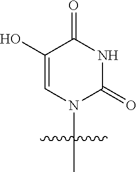 [SMILES: *n1cc(O)c(=O)[nH]c1=O] |
| VIII7 | 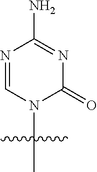 [SMILES: *n1cnc(N)nc1=O] |
| VIII8 | 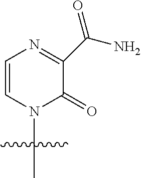 [SMILES: *n1ccnc(C(N)=O)c1=O] |
| VIII9 | 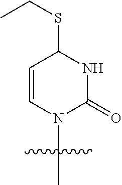 [SMILES: *N1C=CC(SCC)NC1=O] |
| VIII10 | 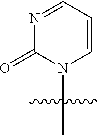 [SMILES: *n1cccnc1=O] |
| VIII11 | 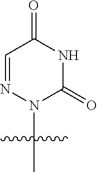 [SMILES: *n1ncc(=O)[nH]c1=O] |
| VIII12 | 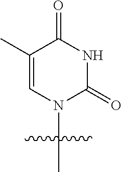 [SMILES: *n1cc(C)c(=O)[nH]c1=O] |
| VIII13 | 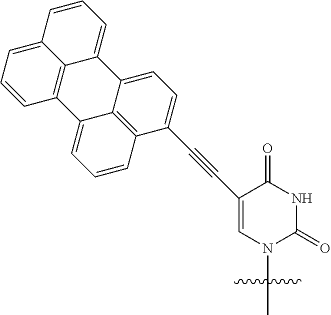 [SMILES: *n1cc(C#Cc2ccc3c4cccc5cccc(c6cccc2c63)c54)c(=O)[nH]c1=O] |
| VIII14 | 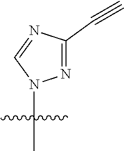 [SMILES: *n1cnc(C#C)n1] |
| VIII15 | 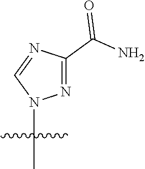 [SMILES: *n1cnc(C(N)=O)n1] |
| VIII16 | 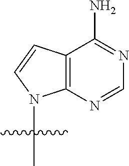 [SMILES: *n1ccc2c(N)ncnc21] |
| VIII17 | 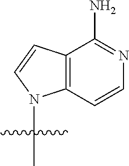 [SMILES: *n1ccc2c(N)nccc21] |
| VIII18 | 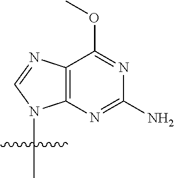 [SMILES: *n1cnc2c(OC)nc(N)nc21] |
| VIII19 | 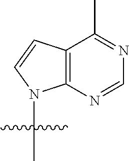 [SMILES: *n1ccc2c(C)ncnc21] |
| VIII20 | 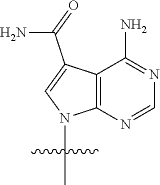 [SMILES: *n1cc(C(N)=O)c2c(N)ncnc21] |
| VIII21 | 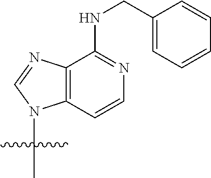 [SMILES: *n1cnc2c(NCc3ccccc3)nccc21] |
| VIII22 | 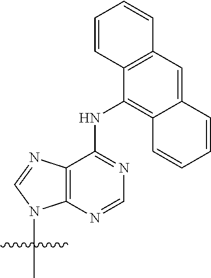 [SMILES: *n1cnc2c(Nc3c4ccccc4cc4ccccc34)ncnc21] |
| VIII23 | 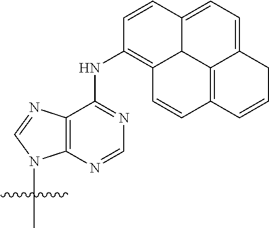 [SMILES: *n1cnc2c(NC3=C4C=CC5=C6C(=CC=C(C=C3)C46)CC=C5)ncnc21] |

In some embodiments, provided herein is a compound represented by the structure of Formula VII1 or a pharmaceutically acceptable salt thereof.

In some embodiments, provided herein is a compound represented by the structure of Formula VIII2 or a pharmaceutically acceptable salt thereof.

In some embodiments, provided herein is a compound represented by the structure of Formula VIII3 or a pharmaceutically acceptable salt thereof.

In some embodiments, provided herein is a compound represented by the structure of Formula VIII4 or a pharmaceutically acceptable salt thereof.

In some embodiments, provided herein is a compound represented by the structure of Formula VIII5 or a pharmaceutically acceptable salt thereof.

In some embodiments, provided herein is a compound represented by the structure of Formula VIII6 or a pharmaceutically acceptable salt thereof.

In some embodiments, provided herein is a compound represented by the structure of Formula VIII7 or a pharmaceutically acceptable salt thereof.

In some embodiments, provided herein is a compound represented by the structure of Formula VIII8 or a pharmaceutically acceptable salt thereof.

In some embodiments, provided herein is a compound represented by the structure of Formula VIII9 or a pharmaceutically acceptable salt thereof.

In some embodiments, provided herein is a compound represented by the structure of Formula VIII10 or a pharmaceutically acceptable salt thereof.

In some embodiments, provided herein is a compound represented by the structure of Formula VIII11 or a pharmaceutically acceptable salt thereof.

In some embodiments, provided herein is a compound represented by the structure of Formula VIII12 or a pharmaceutically acceptable salt thereof.

In some embodiments, provided herein is a compound represented by the structure of Formula VIII13 or a pharmaceutically acceptable salt thereof.

In some embodiments, provided herein is a compound represented by the structure of Formula VIII14 or a pharmaceutically acceptable salt thereof.

In some embodiments, provided herein is a compound represented by the structure of Formula VIII15 or a pharmaceutically acceptable salt thereof.

In some embodiments, provided herein is a compound represented by the structure of Formula VIII16 or a pharmaceutically acceptable salt thereof.

In some embodiments, provided herein is a compound represented by the structure of Formula VIII17 or a pharmaceutically acceptable salt thereof.

In some embodiments, provided herein is a compound represented by the structure of Formula VIII18 or a pharmaceutically acceptable salt thereof.

In some embodiments, provided herein is a compound represented by the structure of Formula VIII19 or a pharmaceutically acceptable salt thereof.

In some embodiments, provided herein is a compound represented by the structure of Formula VIII20 or a pharmaceutically acceptable salt thereof.

In some embodiments, provided herein is a compound represented by the structure of Formula VIII21 or a pharmaceutically acceptable salt thereof.

In some embodiments, provided herein is a compound represented by the structure of Formula VIII22 or a pharmaceutically acceptable salt thereof.

In some embodiments, provided herein is a compound represented by the structure of Formula VIII23 or a pharmaceutically acceptable salt thereof.

In some embodiments, provided herein is a ddh- or a deoxy-ddh-compound represented by the structure of Formula IX

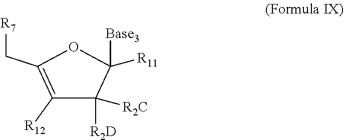

or a pharmaceutically acceptable salt thereof,
wherein
R₁₁ is H, or —CN;
R₂C is an alkyl or an alkyne-R₉;
R₂D is an alkyl or an alkyne-R₁₀;
R₁₂ is H or an alkyl;
R₇ is OH,

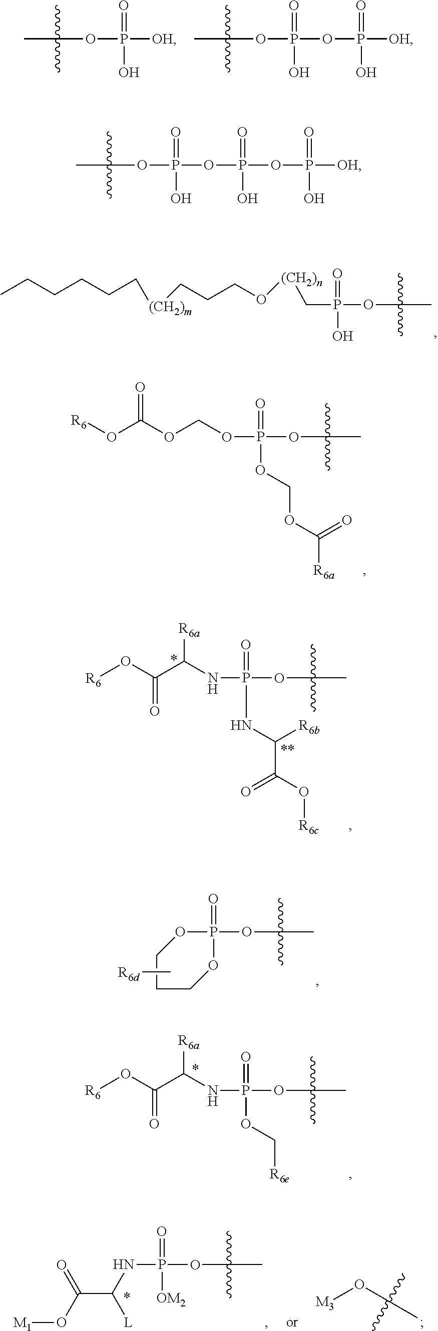

M₁ is a branched or linear alkyl;
L is a side chain of a natural or unnatural amino acid;
M₂ is substituted or unsubstituted aryl, or substituted or unsubstituted heteroaryl;
M₃ is

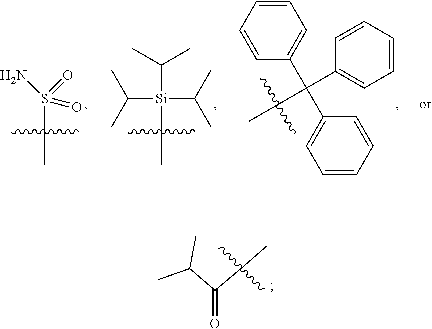

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl; R₉, and R₁₀ are each independently an alkyl;
Base₃ is a C-linked base, wherein the C-linked base comprises:

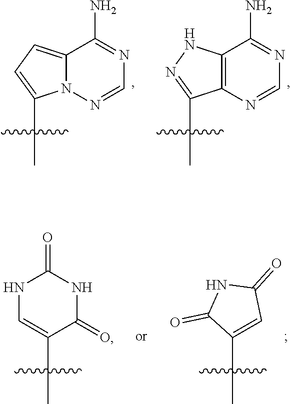 [SMILES: *c1ccc2c(N)ncnn12 ; *c1n[nH]c2c(N)ncnc12 ; *c1c[nH]c(=O)[nH]c1=O ; "or" ; *C1=CC(=O)NC1=O ; ";"]

and

In some embodiments, provided herein is a compound represented by the structure of Formula IX

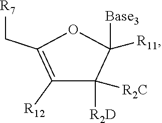

wherein Formula IX is represented by Formulas IX1-IX4 or a pharmaceutically acceptable salt thereof,
wherein
R₁₁, R₂C, R₂D, R₁₂, R₆, R₆ₐ, R_{6b}, R_{6c}, R_{6d}, R₆ₑ, R₇, R₉, R₁₀, n, m, M₁, M₂, M₃ and L are as defined in Formula IX, and Base₃ is a C-linked base represented by the following structure in Table 9.

TABLE 9
C-linked base- based ddh-or deoxy ddh-analog compounds.

| Formula number | Base₃- C-linked base |
|---|---|
| IX1 | 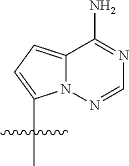 [SMILES: *c1ccc2c(N)ncnn12] |
| IX2 | 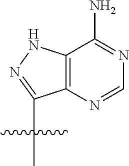 [SMILES: *c1n[nH]c2c(N)ncnc12] |
| IX3 | 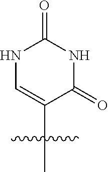 [SMILES: *c1c[nH]c(=O)[nH]c1=O] |
| IX4 | 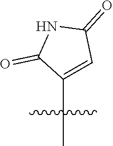 [SMILES: *C1=CC(=O)NC1=O] |

In some embodiments, provided herein is a compound represented by the structure of Formula IX1 or a pharmaceutically acceptable salt thereof.

In some embodiments, provided herein is a compound represented by the structure of Formula IX2 or a pharmaceutically acceptable salt thereof.

In some embodiments, provided herein is a compound represented by the structure of Formula IX3 or a pharmaceutically acceptable salt thereof.

In some embodiments, provided herein is a compound represented by the structure of Formula IX4 or a pharmaceutically acceptable salt thereof.

In one aspect, provided herein is a ddh- or a deoxy-ddh-compound represented by the structure of Formula X

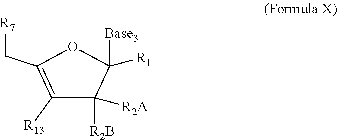

or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₁₃ is a small alkyl(C1-C6);
R₇ is OH,

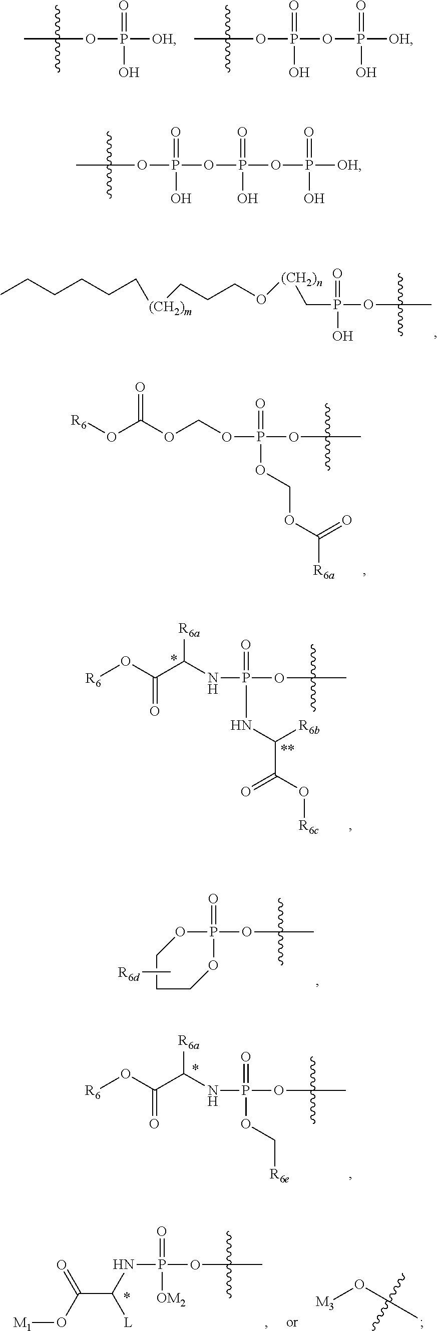

M₁ is a branched or linear alkyl;
L is a side chain of a natural or unnatural amino acid;
M₂ is substituted or unsubstituted aryl;
M₃ is

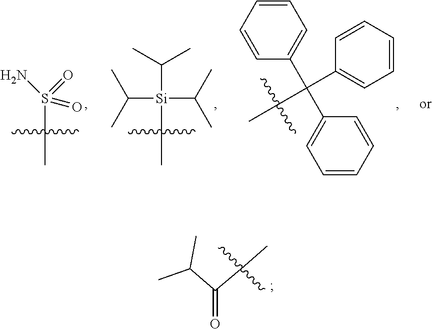

n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl; and
Base₃ is a C-linked base, wherein the C-linked base comprises:

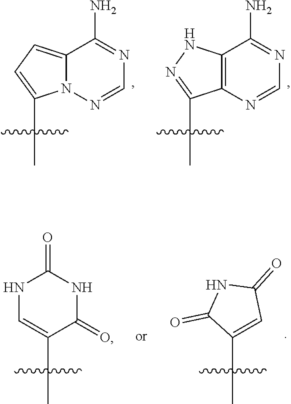 [SMILES: *c1ccc2c(N)ncnn12 ; *c1n[nH]c2c(N)ncnc12 ; *c1c[nH]c(=O)[nH]c1=O ; "or" ; *C1=CC(=O)NC1=O ; "."]

In some embodiments, provided herein is a compound represented by the structure of Formula X

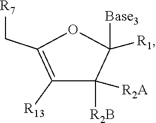

wherein Formula X is represented by Formulas X1-X4 or a pharmaceutically acceptable salt thereof,
wherein
R₁, R₂A, R₂B, R₁₃, R₆, R₆ₐ, R_{6b}, R_{6c}, R_{6d}, R₆ₑ, R₇, R₈, R₉, R₁₀, n, m, M₁, M₂, M₃ and L are as defined in Formula X, and Base₃ is a C-linked base represented by the following structure in Table 10.

TABLE 10
C-linked base- based ddh-or deoxy ddh-analog compounds

| Formula number | Base₃- C-linked base |
|---|---|
| X1 | 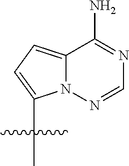 [SMILES: *c1ccc2c(N)ncnn12] |
| X2 | 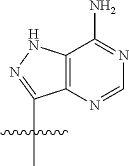 [SMILES: *c1n[nH]c2c(N)ncnc12] |
| X3 | 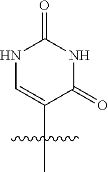 [SMILES: *c1c[nH]c(=O)[nH]c1=O] |
| X4 | 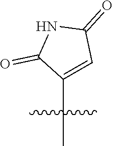 [SMILES: *C1=CC(=O)NC1=O] |

In some embodiments, provided herein is a compound represented by the structure of Formula X1 or a pharmaceutically acceptable salt thereof.

In some embodiments, provided herein is a compound represented by the structure of Formula X2 or a pharmaceutically acceptable salt thereof.

In some embodiments, provided herein is a compound represented by the structure of Formula X3 or a pharmaceutically acceptable salt thereof.

In some embodiments, provided herein is a compound represented by the structure of Formula X4 or a pharmaceutically acceptable salt thereof.

In some embodiment, provided herein is a compound of Formula X represented by the structure of Compound 14

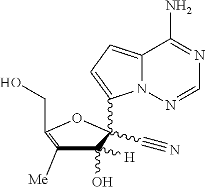

or a pharmaceutically acceptable salt thereof. In another embodiment, Compound 14 is (2R,3R)-2-(4-aminopyrrolo[2,1-f][1,2,4]triazin-7-yl)-3-hydroxy-5-(hydroxymethyl)-4-methyl-2,3-dihydrofuran-2-carbonitrile or a pharmaceutically acceptable salt thereof. In another embodiment, C1′ of Compound 14 is in R configuration. In another embodiment, C1′ of Compound 14 is in S configuration. In another embodiment, C2′ of Compound 14 is in R configuration. In another embodiment, C2′ of Compound 14 is in S configuration. In another embodiment, compound 14 is obtain as racemic mixture.

In some embodiments, R₁ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, X4, A14, A15, or A16 is —H, —CN, —N₃, alkyne-R₈, or an alkyl. In another embodiment, R₁ is H. In another embodiment, R₁ is —CN. In another embodiment, R₁ is —N₃. In another embodiment, R₁ is alkyne-R₈. In another embodiment, R₁ is an alkyl. In another embodiment, R₁ is C1-C3 alkyl. In another embodiment, R₁ is C1-C5 alkyl. In another embodiment, R₁ is C1-C8 alkyl.

In one embodiment, R₁ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, X4, A14, A15, or A16 is alkyne-R₈. In another embodiment, R₈ is an alkyl. In another embodiment, R₈ is C1-C3 alkyl. In another embodiment, R₈ is C1-C5 alkyl. In another embodiment, R₈ is C1-C8 alkyl.

In some embodiments, R₁₁ of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 is H or —CN. In another embodiment, R₁₁ is H. In another embodiment, R₁₁ is —CN.

In some embodiments, R₂A of Formula I, I1, I2, I3, I4, I5, I6, II, I11, I12, I13, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, or X4, is H, —OH, an alkyl, a halo, an alkyne-R₉, or O—C(O)OR₉. In another embodiment, R₂A is H. In another embodiment, R₂A is OH. In another embodiment, R₂A is an alkyl. In another embodiment, R₂A is a halo. In another embodiment, R₂A is an alkyne-R₉. In another embodiment, R₂A is O—C(O)OR₉. In another embodiment, R₂A is C1-C3 alkyl. In another embodiment, R₂A is C1-C5 alkyl. In another embodiment, R₂A is C1-C8 alkyl.

In one embodiment, R₂A of Formula I, I1, I2, I3, I4, I5, I6, II, II1, II2, II3, II4, II5, II6, II7, II8, II9, II10, II1, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, or X4 is O—C(O)OR₉ or an alkyne-R₉. In another embodiment, R₉ is an alkyl. In another embodiment, R₉ is C1-C3 alkyl. In another embodiment, R₉ is C1-C5 alkyl. In another embodiment, R₉ is C1-C8 alkyl.

In some embodiments, R₂B of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, or X4 is H, OH, an alkyl, a halo, an alkyne-R₁₀, or O—C(O)OR₁₀. In another embodiment, R₂B is H. In another embodiment, R₂B is OH. In another embodiment, R₂B is an alkyl. In another embodiment, R₂B is a halo. In another embodiment, R₂B is an alkyne-R₁₀. In another embodiment, R₂B is O—C(O)OR₁₀. In another embodiment, R₂B is C1-C3 alkyl. In another embodiment, R₂B is C1-C5 alkyl. In another embodiment, R₂B is C1-C8 alkyl.

In one embodiment, R₂B of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, or VI4, X, X1, X2, X3, or X4 is an alkyne-R₁₀, or O—C(O)OR₁₀. In another embodiment, R₁₀ is an alkyl. In another embodiment, R₁₀ is C1-C3 alkyl. In another embodiment, R₁₀ is C1-C5 alkyl. In another embodiment, R₁₀ is C1-C8 alkyl.

In some embodiments, R₂C of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 is an alkyl, or an alkyne-R₉. In another embodiment, R₂C an alkyl. In another embodiment, R₂C is an alkyne-R₉. In another embodiment, R₂C is C1-C3 alkyl. In another embodiment, R₂C is C1-C5 alkyl. In another embodiment, R₂Cis C1-C8 alkyl. In another embodiment, R₂C is C2-C5 alkyl. In another embodiment, R₂C is C2-C8 alkyl.

In one embodiment, R₂C of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 is alkyne-R₉. In another embodiment, R₉ is an alkyl. In another embodiment, R₉ is C1-C3 alkyl. In another embodiment, R₉ is C1-C5 alkyl. In another embodiment, R₉ is C1-C8 alkyl.

In some embodiments, R₂D of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 is an alkyl, or an alkyne-R₁₀. In another embodiment, R₂D an alkyl. In another embodiment, R₂D is an alkyne-R₁₀. In another embodiment, R₂D is C1-C3 alkyl. In another embodiment, R₂D is C1-C5 alkyl. In another embodiment, R₂D is C1-C8 alkyl. In another embodiment, R₂D is C2-C5 alkyl. In another embodiment, R₂D is C2-C8 alkyl.

In one embodiment, R₂D of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 is an alkyl, or an alkyne-R₁₀. In another embodiment, R₁₀, is an alkyl. In another embodiment, R₁₀ is C1-C3 alkyl. In another embodiment, R₁₀ is C1-C5 alkyl. In another embodiment, R₁₀ is C1-C8 alkyl. In another embodiment, R₁₀ is C2-C5 alkyl.

In some embodiment, if R₂A and R₂B of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, X, X1, X2, X3, or X4 are different then C2′ is chiral. In another embodiment, C2′ is in R-configuration. In another embodiment, C2′ is in R-configuration. S-configuration

In some embodiments, if R₂C and R₂D of Formula VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, or IX4 are different then C2′ is chiral. In another embodiment, C2′ is in R-configuration. In another embodiment, C2′ is in R-configuration. S-configuration

In some embodiments, R₃ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, or VI4 is H, a halo or an alkyl. In another embodiment, R₃ is H. In another embodiment, R₃ is a halo. In another embodiment, R₃ is an alkyl. In another embodiment, R₃ is a methyl. In another embodiment, R₃ is C1-C3 alkyl. In another embodiment, R₃ is C1-C5 alkyl. In another embodiment, R₃ is C1-C8 alkyl. In another embodiment, R₃ is C1. In another embodiment, R₃ is Br. In another embodiment, R₃ is I. In another embodiment, R₃ is F.

In some embodiments, when R₃ is a halo, then R₁ is H, —CN, —N₃, an alkyne-R₈, or an alkyl, for Formula IV, V and VI. In another embodiment, when R₃ is a halo, then R₁ is H. In another embodiment, when R₃ is a halo, then R₁ is —CN. In another embodiment, when R₃ is a halo, then R₁ is —N₃. In another embodiment, when R₃ is a halo, then R₁ is an alkyne-R₈. In another embodiment, when R₃ is a halo, then R₁ is an alkyl. In another embodiments, R₈ is an alkyl.

In some embodiments, for Formula IV, V and VI when R₃ is H or an alkyl, then R₁ is —N₃, an alkyne-R₈, or an alkyl

In another embodiment, when R₃ is H, then R₁ is —N₃. In another embodiment, when R₃ is H, then R₁ is an alkyne-R₈. In another embodiment, when R₃ is H, then R₁ is an alkyl. In another embodiment, when R₃ is an alkyl, then R₁ is —N₃. In another embodiment, when R₃ is an alkyl, then R₁ is an alkyne-R₈. In another embodiment, when R₃ is an alkyl, then R₁ is an alkyl.

In some embodiments, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, or III4 is

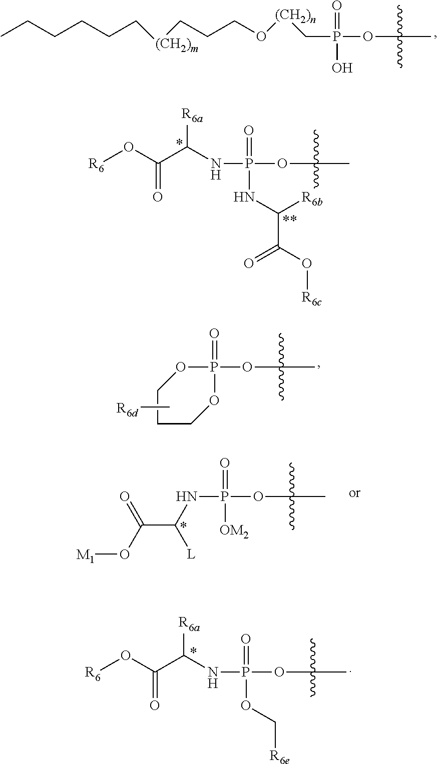

In another embodiment, R₅ is

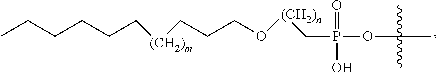

In another embodiment, R₅ is

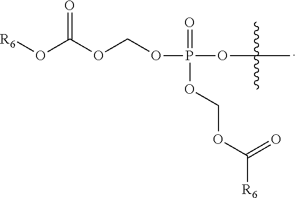

In another embodiment, R₅ is

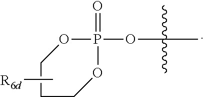

In another embodiment, R₅ is

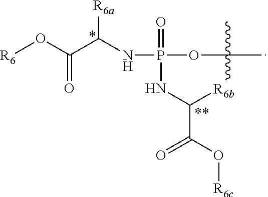

In another embodiment, R₅ is

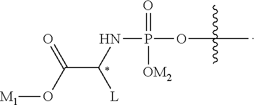

In another embodiment, R₅ is

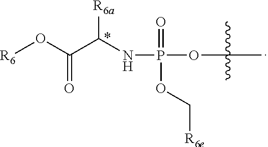

In some embodiments, R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is OH,

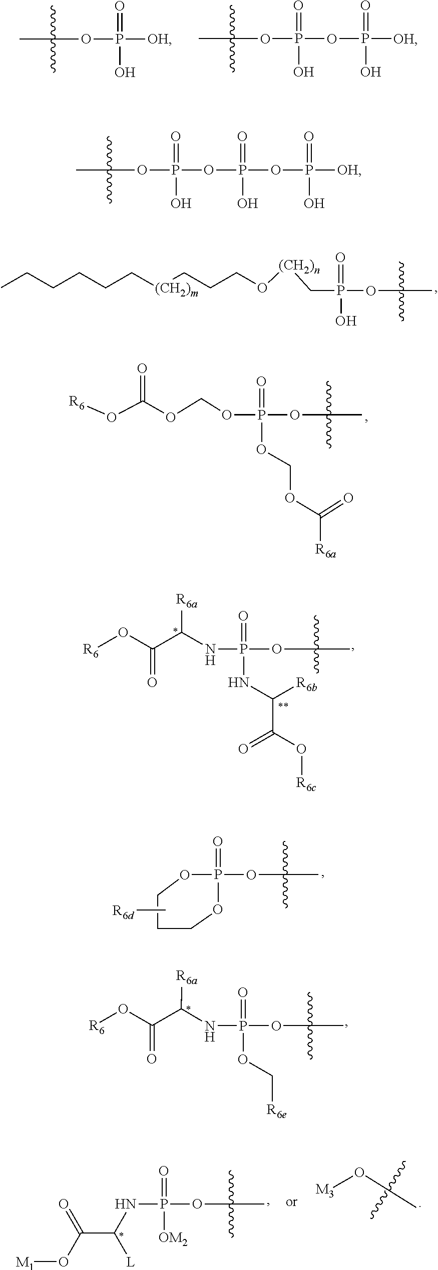

In another embodiment, R₇ is OH. In another embodiment, R₇ is

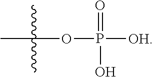 [SMILES: *OP(=O)(O)O]

In another embodiment, R₇ is

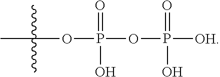 [SMILES: *OP(=O)(O)OP(=O)(O)O]

In another embodiment, R₇ is

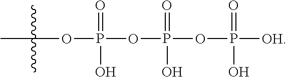 [SMILES: *OP(=O)(O)OP(=O)(O)OP(=O)(O)O]

In another embodiment, R₇ is

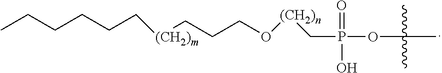

In another embodiment, R₇ is

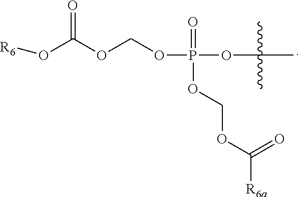

In another embodiment, R₇ is

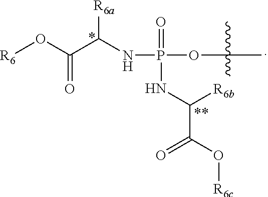

In another embodiment, R₇ is

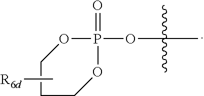

In another embodiment, R₇ is

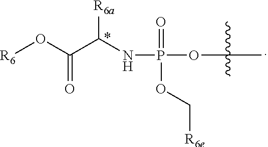

In another embodiment, R₇ is

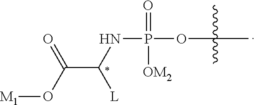

In another embodiment, R₇ is or

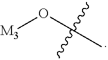

In one embodiment, R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

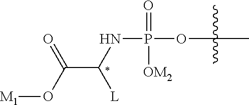

In another embodiment, M₁ is a branched or linear alkyl. In another embodiment, M₁ is branched alkyl. In another embodiment, M₁ is branched C3-C8alkyl. In another embodiment, M₁ is branched C3-C10alkyl. In another embodiment, M₁ is an alkyl. In another embodiment, M₁ is C1-C5alkyl. In another embodiment, M₁ o is C1-C8alkyl. In another embodiment, M₁ is

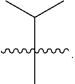 [SMILES: *C(C)C]

In another embodiment, M₁ is

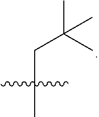 [SMILES: *CC(C)(C)C]

In another embodiment, M₁ is

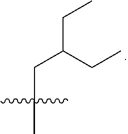 [SMILES: *CC(CC)CC]

In one embodiment, M₂ is substituted or unsubstituted aryl, or substituted or unsubstituted heteroaryl. In another embodiment, M₂ is

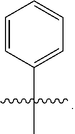 [SMILES: *c1ccccc1]

In another embodiment, M₂ is

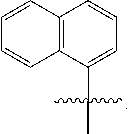 [SMILES: *c1cccc2ccccc12]

In another embodiment, M₂ is an unsubstituted aryl. In another embodiment, M₂ is a substituted aryl. In another embodiment, M₂ is an unsubstituted heteroaryl. In another embodiment, M₂ is a substituted heteroaryl. In one embodiment, L is a side chain of a natural or unnatural amino acid. In another embodiment, L is methyl. In one embodiment, R₇ is

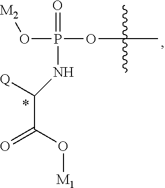

wherein M₁ is

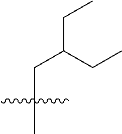 [SMILES: *CC(CC)CC]

### and M₂ is

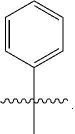 [SMILES: *c1ccccc1]

In one embodiment, R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

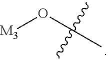

In another embodiment, M₃ is

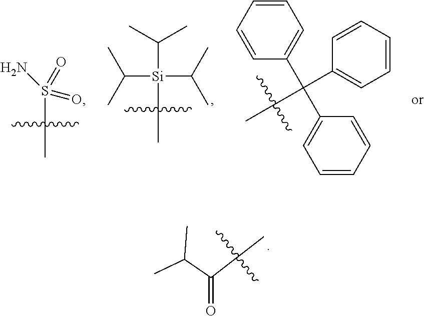

In another embodiment, M₃ is

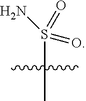 [SMILES: *S(N)(=O)=O]

In another embodiment, M₃ is

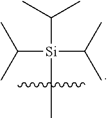 [SMILES: *[Si](C(C)C)(C(C)C)C(C)C]

In another embodiment, M₃ is

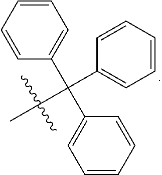 [SMILES: *C(c1ccccc1)(c1ccccc1)c1ccccc1]

In another embodiment, M₃ is

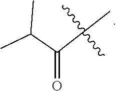 [SMILES: *C(=O)C(C)C]

In one embodiment, the chiral carbon of

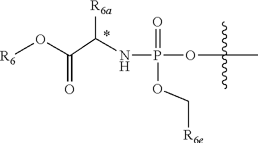

is S or R. In another embodiment, the chiral carbon of

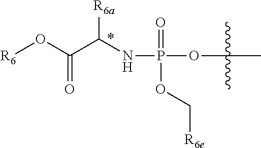

is S. In another embodiment, the chiral carbon of

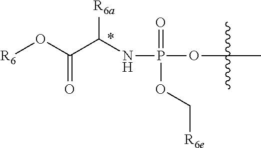

is R. In one embodiment, the chiral carbon (*) and (**) of

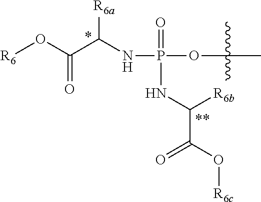

are each independently S or R. In another embodiment, the chiral carbon (*) and (**) of

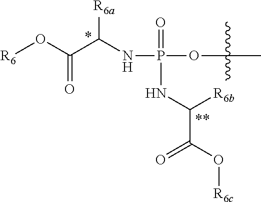

are each independently S. In another embodiment, the chiral carbon (*) and (**) of

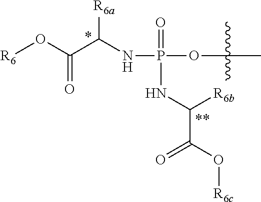

are each independently R.

In one embodiment, the chiral carbon of

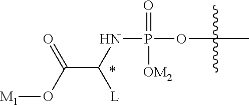

is S or R. In another embodiment, the chiral carbon of

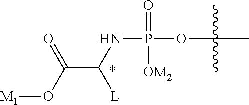

is S. In another embodiment, the chiral carbon of

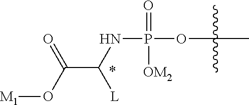

### is R.

In some embodiments, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

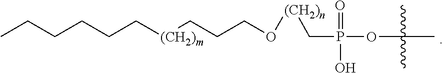

In another embodiment, n is an integer between 1 to 4. In another embodiment, n is 1. In another embodiment, n is 2. In another embodiment, n is 3. In another embodiment, n is 4. In another embodiment, m is an integer between 1 to 4. In another embodiment, m is 1. In another embodiment, m is 2. In another embodiment, m is 3. In another embodiment, m is 4.

In some embodiments, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, or III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

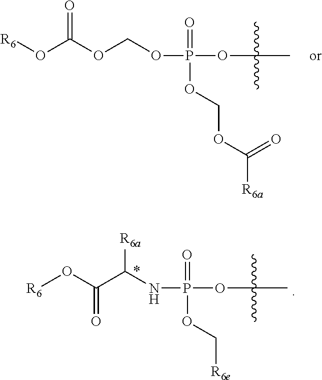

In another embodiment, R₆ is branched or linearlinear alkyl. In another embodiment, R₆ is branched alkyl. In another embodiment, R₆ is C3-C8 branched alkyl. In another embodiment, R₆ is C3-C10 branched alkyl. In another embodiment, R₆ is linear alkyl. In another embodiment, R₆ is C1-C3 alkyl. In another embodiment, R₆ is C1-C5 alkyl. In another embodiment, R₆ is C1-C8 alkyl.

In some embodiments, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

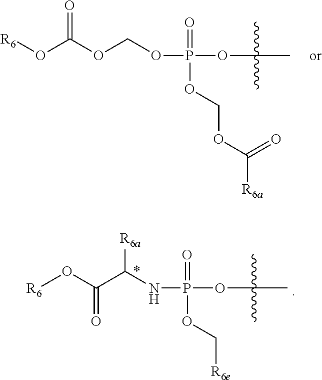

In another embodiment, R₆ₐ is branched or linear alkyl. In another embodiment, R₆ₐ is branched alkyl. In another embodiment, R₆ₐ is C3-C8 branched alkyl. In another embodiment, R₆ₐ is C3-C10 branched alkyl. In another embodiment, R₆ₐ is linear alkyl. In another embodiment, R₆ₐ is C1-C3 alkyl. In another embodiment, R₆ₐ is C1-C5 alkyl. In another embodiment, R₆ₐ is C1-C8 alkyl.

In one embodiment, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

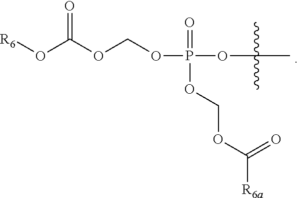

In another embodiment, R_{6b} is branched or linear alkyl. In another embodiment, R_{6b} is branched alkyl. In another embodiment, R_{6b} is C3-C8 branched alkyl. In another embodiment, R_{6b} is C3-C10 branched alkyl. In another embodiment, R_{6b} is linear alkyl. In another embodiment, R_{6b} is C1-C3 alkyl. In another embodiment, R_{6b} is C1-C5 alkyl. In another embodiment, R_{6b} is C1-C8 alkyl.

In one embodiment, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

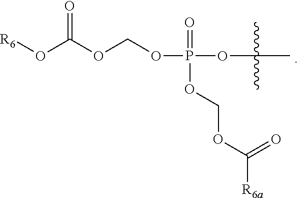

In another embodiment, R_{6c} is branched or linear alkyl. In another embodiment, R_{6c} is branched alkyl. In another embodiment, R_{6c} is C3-C8 branched alkyl. In another embodiment, R_{6c} is C3-C10 branched alkyl. In another embodiment, R_{6c} is linear alkyl. In another embodiment, R_{6c} is C1-C3 alkyl. In another embodiment, R_{6c} is C1-C5 alkyl. In another embodiment, R_{6c} is C1-C8 alkyl.

In one embodiment, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

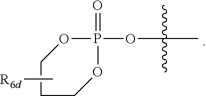

In another embodiment, R_{6d} is H or an alkyl. In another embodiment, R_{6d} is H. In another embodiment, R_{6d} is C1-C3 alkyl. In another embodiment, R_{6d} is C1-C5 alkyl. In another embodiment, R_{6d} is C1-C8 alkyl.

In one embodiment, R₅ of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, or R₇ of Formula IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15 or A16 is

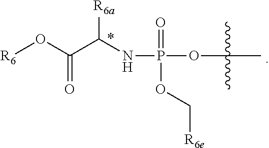

In another embodiment, R₆ₑ is a haloalkyl. In another embodiment, R₆ₑ is CF₃. In another embodiment, R₆ₑ is halo-C1-C3 alkyl. In another embodiment, R₆ₑ is halo-C1-C5 alkyl. In another embodiment, R₆ₑ is halo-C1-C8 alkyl.

In some embodiments, R₁₂ of VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, and IX4, is H or an alkyl. In another embodiment, R₁₂ is H. In another embodiment, R₁₂ is C1-C3 alkyl. In another embodiment, R₁₂ is C1-C5 alkyl. In another embodiment, R₁₂ is C1-C8 alkyl. In another embodiment, R₁₂ is C1-C12 alkyl.

In some embodiments, R₁₃ of Formula X, A14, or A15, is a small alkyl(C1-C6). In another embodiment, R₁₃ is C1-C6 alkyl. In another embodiment, R₁₃ is C1-C3 alkyl. In another embodiment, R₁₃ is C1-C5 alkyl. In another embodiment, R₁₃ is C2-C6 alkyl. In another embodiment, R₁₃ is methyl.

In some embodiment, C1′ or C2′ of the compounds provided herein are each can be either or in the R- or S-configuration.

In some embodiments, C1′ of a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VIII, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A15, A16, A14, 14, 15 or 16 comprises a chiral carbon. In another embodiment, the chiral carbon C1′ is an S isomer. In another embodiment, the chiral carbon C1′ is an R isomer. In some embodiment, C1′ or C2′ are not chiral.

In some embodiments, a compound described herein is a prodrug. In some embodiments, the ddh- or deoxy-ddh-prodrug compound or active metabolites thereof can be synthesized and administered directly to cells or a subject. Upon entering the cells, these ddh- or deoxy-ddh-compounds can be further phosphorylated by one or more cellular kinases to produce the active metabolites that inhibit DNA or RNA replication, or both. In one embodiment, the ddh- or deoxy-ddh-compound comprise a prodrug.

One skilled in the art would appreciate that the term “prodrug” may in certain embodiments, encompass any compound that when administered to a biological system could be converted into an active compound or metabolite thereof as a result of spontaneous chemical reaction(s), enzyme catalyzed chemical reaction(s), photolysis, and/or metabolic chemical reaction(s). The active compound or metabolites in the present disclosure are the DNA or RNA chain terminators or both, which in some embodiments may be synthesized using methods known in the art.

In certain embodiments, the ddh- and deoxy-ddh-compounds described herein can be used to block cellular DNA/RNA replication or to treat a disease in a subject. In some embodiments, the ddh- and deoxy-ddh-compounds are administered to cells in a form that can enter the cells (e.g., nucleoside form or prodrug form). Once inside the cells, these the ddh- and deoxy-ddh-compounds can be converted (e.g., phosphorylation by one or more cellular kinases).

In some embodiments, a prodrug facilitates the crossing of the plasma membrane of a cell by the compound. In some embodiments, the prodrug form of a compound facilitates passive diffusion through the cell membrane by masking negative charge until the compound is within the cell.

In some embodiments, a prodrug comprises a protective chemical group. In some embodiments, a protective chemical group comprises

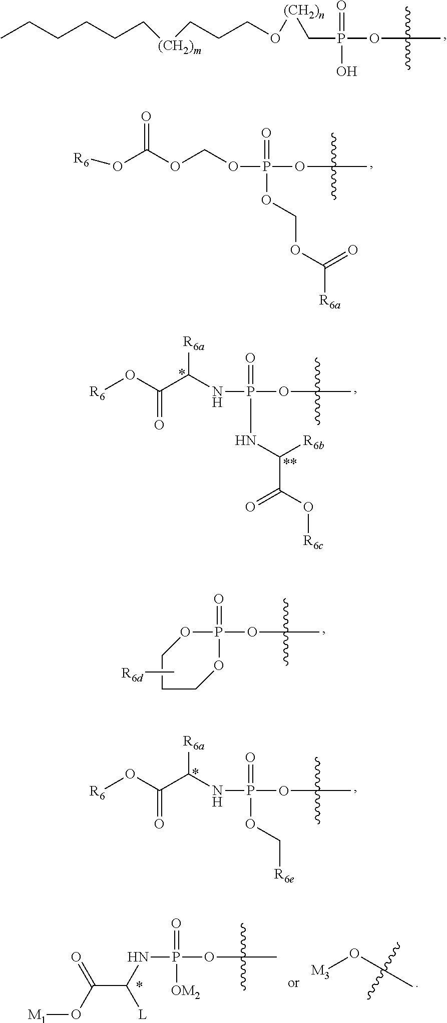

M₁ is a branched or unbranched alkyl;
L is a side chain of a natural or unnatural amino acid;
M₂ is substituted or unsubstituted aryl, or substituted or unsubstituted heteroaryl;
M₃ is

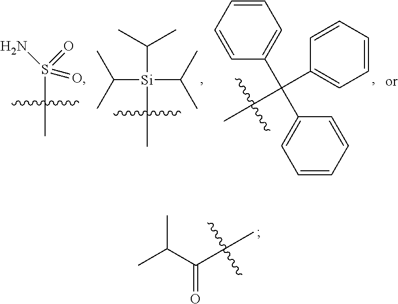

n is an integer between 1-4;
m is an integer between 1-4;
R₆, R₆ₐ, R_{6b}, and R_{6c} are each independently a branched or unbranched alkyl;
R_{6d} is H or an alkyl;
R₆ₑ is a haloalkyl;
In another embodiment, the protective chemical group is

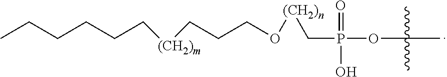

In another embodiment, the protective chemical group is

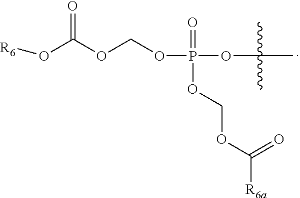

In another embodiment, the protective chemical group is

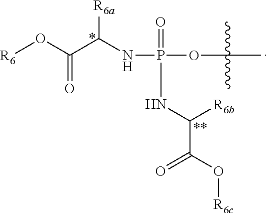

In another embodiment, the protective chemical group is

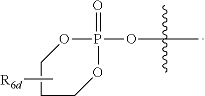

In another embodiment, the protective chemical group is

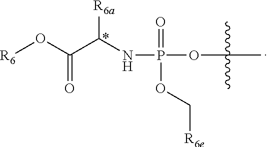

In another embodiment, the protective chemical group
is

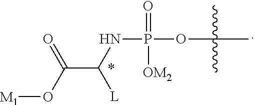

In another embodiment, the protective chemical group is

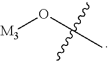

In some embodiments, the present disclosure includes all forms of prodrugs that are covalently modified analogs or latent forms of the therapeutically active metabolites (the DNA/RNA chain terminators). In one embodiment, the prodrugs comprise the ddh- or deoxy-ddh-compounds described herein or modified structures thereof. The prodrug form can serve to enhance solubility, absorption and lipophilicity to optimize drug delivery, bioavailability and efficacy of the comprise the ddh- or deoxy-ddh-compounds. In one embodiment, a prodrug comprises the comprise the ddh- or deoxy-ddh-compounds with a chemical structure that can be oxidized, reduced, aminated, deaminated, esterified, de-esterified, alkylated, dealkylated, acylated, deacylated, phosphorylated, dephosphorylated, photolyzed, hydrolyzed, or other functional group change or conversion to produce the therapeutically active metabolite (the DNA/RNA chain terminators), or produce the active metabolite that can be transported across cell membrane. Enzymes which are capable of enzymatic activation of prodrugs include, but are not limited to, amidases, esterases, microbial enzymes, phospholipases, cholinesterases, and phosphatases. Designs and uses of prodrugs are generally known in the art, e.g., Bundgaard, Hans, “Design and Application of Prodrugs” in Textbook of Drug Design and Development (1991), P. Krogsgaard-Larsen and H. Bundgaard, Eds. Harwood Academic Publishers.

As used herein, the term alkyl, used alone or as part of another group, refers, in one embodiment, to a “C₁ to C₁₈ alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups containing from 1 to 6 carbon atoms (C1 to C₆ alkyls), or alkyl groups containing from 1 to 4 carbon atoms (C₁ to C₄ alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like.

In another embodiment, the term alkyl used alone or as part of another group, refers, in one embodiment, to a “C₁ to C₃ alkyl” and denotes linear and branched, saturated or unsaturated (e.g., alkenyl, alkynyl) groups, the latter only when the number of carbon atoms in the alkyl chain is greater than or equal to two, and can contain mixed structures. In another embodiment, the term alkyl used alone or as part of another group, refers, in one embodiment, to a “C₁ to C₅ alkyl” and denotes linear and branched, saturated or unsaturated (e.g., alkenyl, alkynyl) groups, the latter only when the number of carbon atoms in the alkyl chain is greater than or equal to two, and can contain mixed structures. In another embodiment, the term alkyl used alone or as part of another group, refers, in one embodiment, to a “C₁ to C₆ alkyl” and denotes linear and branched, saturated or unsaturated (e.g., alkenyl, alkynyl) groups, the latter only when the number of carbon atoms in the alkyl chain is greater than or equal to two, and can contain mixed structures. In another embodiment, the term alkyl used alone or as part of another group, refers, in one embodiment, to a “C₁ to C₈ alkyl” and denotes linear and branched, saturated or unsaturated (e.g., alkenyl, alkynyl) groups, the latter only when the number of carbon atoms in the alkyl chain is greater than or equal to two, and can contain mixed structures. Similarly, the term “C₁ to C₁₂ alkylene” denotes a bivalent radical of 1 to 12 carbons.

As used herein, the term small alkyl, used alone or as part of another group, refers to, in one embodiment, to a “C₁ to C6 alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups containing from 1 to 6 carbon atoms (C₁ to C₆ alkyls), or alkyl groups containing from 1 to 4 carbon atoms (C₁ to C₄ alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like.

The alkyl group (also the small alkyl) can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

The term “haloalkyl” used herein alone or as part of another group, refers to, in some embodiments, to an alkyl group as defined above, which is substituted by one or more halogen atoms, e.g., by F, Cl, Br or I.

The term “alkyne” used herein alone or as part of another group, refers to an alkyl as defined above with at least one triple bond. The “alkyne” in some embodiment, refer to have 2 to 20 carbon atoms (C₂-C₁₈ alkyne). The Alkyne, in some embodiments, is unsubstituted. The Alkyne, in some embodiments, is substituted with one or more substituents selected from the group consisting of aryl, halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

“Alkenyl” is a hydrocarbon containing normal, secondary, tertiary or cyclic carbon atoms with at least one site of unsaturation, i.e., a carbon-carbon, sp² double bond. For example, an alkenyl group can have 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of suitable alkenyl groups include, but are not limited to, ethenyl or vinyl (both having a structure —CH═C2) allyl (—CH₂CH═CH₂), cyclopentenyl (—C₅H₇), and 5-hexenyl (—CH₂CH₂CH₂CH₂CH═CH₂).

“Alkynyl” is a hydrocarbon containing normal, secondary, tertiary or cyclic carbon atoms with at least one site of unsaturation, i.e., a carbon-carbon, sp triple bond. For example, an alkynyl group can have 2 to 20 carbon atoms (i.e., C₂-C₂₀ alkynyl), 2 to 8 carbon atoms (i.e., C₂-C₈ alkyne,), 2 to 6 carbon atoms (i.e., C₂-C₆ alkynyl), or 2 to 4 carbon atoms (i.e., C₂-C₄ alkynyl). Examples of suitable alkynyl groups include, but are not limited to, ethynyl or acetylenic (—C≡CH), propargyl (—CH₂C═CH), and the like.

“Alkylene” refers to a saturated, branched or straight chain or cyclic hydrocarbon radical having two monovalent radical centers derived by the removal of two hydrogen atoms from the same or two different carbon atoms of a parent alkane. For example, an alkylene group can have 1 to 20 carbon atoms, 1 to 10 carbon atoms, or 1 to 6 carbon atoms. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl —OCN, —SCN, —N═C═O, —NCS, —NO, —Na, —OP(═O)(OR*)₂, —P(═O)(OR*)₂, —P(═O)(O⁻)₂, —P(═O)(OH)₂, —P(O)(OR*)(O⁻), —C(═O)R*, —C(═O)X, —C(S)R*, —C(S)OR*, —C(O)SR*, —C(S)SR*, —C(S)NR*₂ or —C(═NR*)NR*₂ groups, where each R* is independently H, alkyl, aryl, arylalkyl, a heterocycle, or a protecting group or prodrug moiety groups. Any substituents can be unsubstituted or further substituted with any one of these aforementioned substituents.

The term “heteroaryl” refers to an aromatic ring system containing from 5-14-member ring having at least one heteroatom in the ring. Non-limiting examples of suitable heteroatoms which can be included in the aromatic ring include oxygen, sulfur, phosphate and nitrogen. Non-limiting examples of heteroaryl rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc. The heteroaryl group can be unsubstituted or substituted through available carbon atoms with one or more groups such as. halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, amido, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl, —OCN, —SCN, —N═C═O, —NCS, —NO, —N₃, —OP(═O)(OR*)₂, —P(═O)(OR*)₂, —P(═O)(O⁻)₂, —P(═O)(OH)₂, —P(O)(OR*)(O⁻), —C(═O)R*, —C(═O)X, —C(S)R*, —C(S)OR*, —C(O)SR*, —C(S)SR*, —C(S)NR*₂ or —C(═NR*)NR*₂ groups, where each R* is independently H, alkyl, aryl arylalkyl, a heterocycle, or a protecting group or prodrug moiety. Any substituents can be unsubstituted or further substituted with any one of these aforementioned substituents.

The term “halogen” or “halo” as used herein refers to —Cl, —Br, —F, or —I groups.

As used herein, the term “amido”, used alone or as part of another group, refers, to the formula —C(═O)NRR′ or —NHCO—R or —N(R)—C(O)—R, wherein R and R′ are each individually is H or an C₁ to C₁₀ alkyl, aryl, cycloalkyl, heterocycle as defined above.

As used herein, the term “side chain of a natural or unnatural amino acid” refers to the side group of each amino acid, such as substituent that is specific to each amino acid, wherein the “side chain” is an organic substituent. The “side chain of an amino acid” comprises H refers to the side chain of Glycine, methyl refers to the side chain of Alanine, benzyl refers to the side chain of Phenylalanine, iso-propyl refers to the side chain of Valine, iso-butyl refers to the side chain of Leucine, sec-butyl refers to the side chain of Isoleucine, —CH₂OH refers to the side chain of Serine,

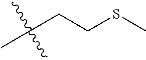 [SMILES: *CCSC]

refers to the side chain of Methionine,

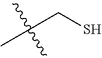 [SMILES: *CS]

refers to the side chain of Cysteine,

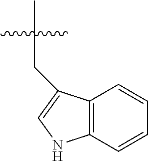 [SMILES: *Cc1c[nH]c2ccccc12]

refers to the side chain of Tryptophan,

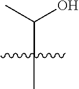 [SMILES: *C(C)O]

refers to the side chain of Threonine, —CH₂CONH₂ refers to the side chain of Asparagine,

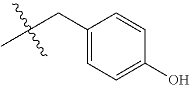 [SMILES: *Cc1ccc(O)cc1]

refers to the side chain of Tyrosine, —CH₂COOH or CH₂COO— refers to the side chain of Aspartic acid, —CH₂CH₂COOH or —CH₂CH₂COO⁻ refers to the side chain of Glutamic acid,
—CH₂CH₂CONH₂ refers to the side chain of Glutamine —CH₂CH₂CH₂NH₂ or —CH₂CH₂CH₂NH₃⁺ refer to the side chain of Lysine,

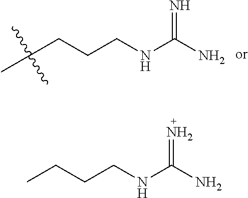

refer to the side chain of Arginine,

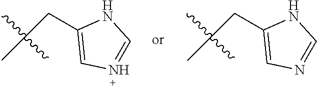 [SMILES: *Cc1cnc[nH]1 ; *Cc1cnc[nH]1]

refer to the side chain of Histidine, —CH₂CH₂CH₂— connected to the N of the R₁ structure refer to the side chain of Proline, —CH₂SeH refer to the side chain of Selenocysteine,

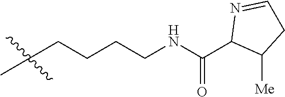 [SMILES: *CCCCNC(=O)C1N=CCC1C]

refer to the side chain of Pyrrolysine.

A skilled artisan would appreciate that “a pharmaceutical acceptable salt” of a compound of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A14, A15, A16, 14, 15 or 16 may in some embodiments be formed by the reaction of a compound of formula I with an acid or base. The term “pharmaceutically acceptable salt” may encompass those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present compounds provided herein.

Suitable pharmaceutically-acceptable salts of amines of compounds provided herein may be prepared from an inorganic acid or from an organic acid. In some embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In some embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of phosphite may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of phosphite may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In some embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

### Synthesis

In some embodiments, provided herein is a process for the preparation of a compound of Formula A16, as represented by the structure of

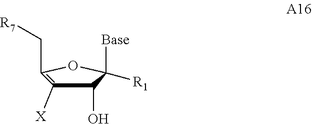

wherein
R₁ is H, —CN, —N₃, an alkyne-R₈, or an alkyl;
X is a halo;
R₇ is OH,

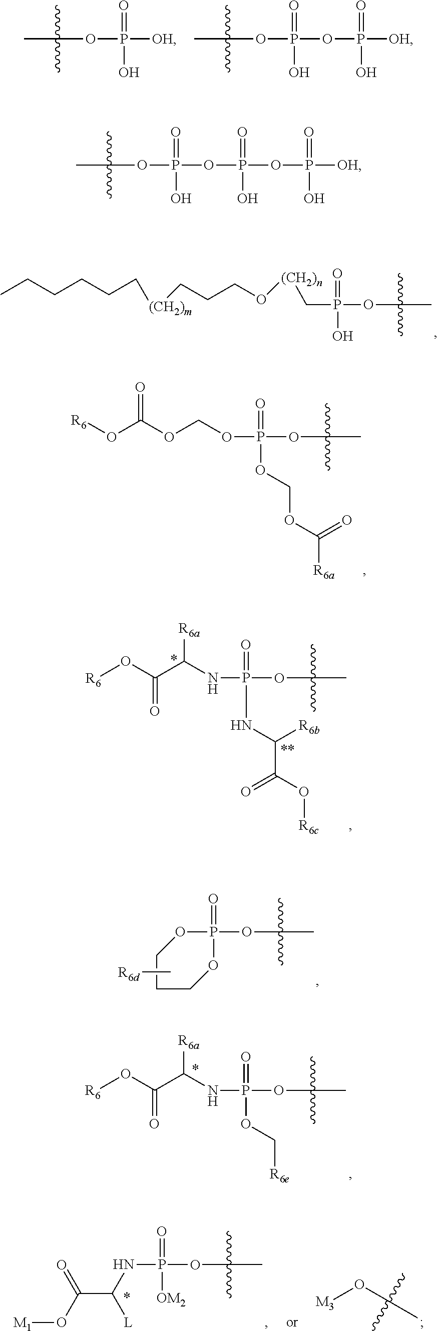

M₁ is a branched or linear alkyl;
L is a side chain of a natural or unnatural amino acid;
M₂ is substituted or unsubstituted aryl, or substituted or unsubstituted heteroaryl;
M₃ is

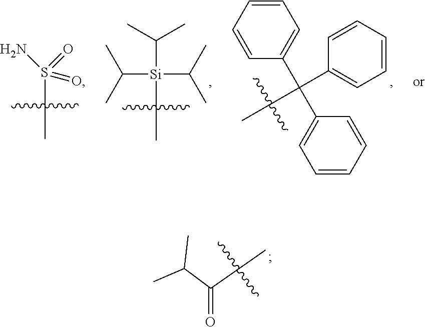

n is an integer between 1-4;
m is an integer between 1-4;
R₆, R₆ₐ, R_{6b}, and R_{6c} are each independently branched or linear alkyl;
R_{6d} is H or an alkyl;
R₆ₑ is a haloalkyl; and
Base is a N-linked natural base, N-linked modified purine or pyrimidine base or C-linked base, as described in Formulas I-VI; wherein A16 is a compound of Formulas I-VI.
wherein the process comprises the steps below.

### Step 1: Preparation of A2

Selectively protecting the OH groups at C3′ and C5′ of a nucleoside, natural or synthetic (for example Compound A1 as shown below), followed by protecting C2′ with an orthogonal protecting group, which optionally will also protect the base (see Greene and Wuts, Greene's Protective Groups in Organic Synthesis, fourth edition, 2006). (as shown in scheme 1)

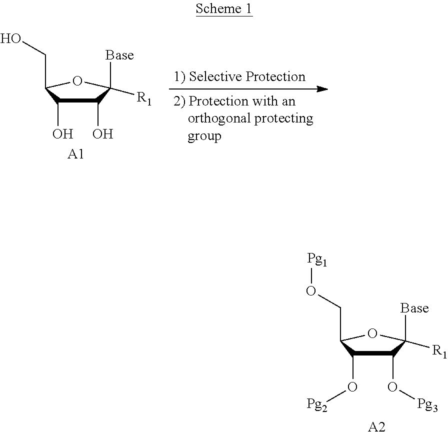

wherein
Pg₁ and Pg₂ are each independently protecting group, or Pg₁ and Pg₂ form 6-10 membered ring;
Pg₃ is an orthogonal protecting group;
Base is N-linked natural base, N-linked modified purine or pyrimidine base or C-linked base; and
R₁ is as defined above.

### Step 2: Preparation of A3

Selectively de-protecting positions C3′ and C5′ to obtain compound A3 (as shown in scheme 2)

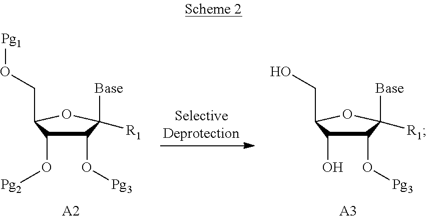

wherein
R₁, Pg₁, Pg₂, Pg₃, and a Base are as defined above.

### Step 3: Preparation of A4

Selectively re-protecting on the OH group at C5′ of compound A3 to obtain compound A4 (as shown in scheme 3)

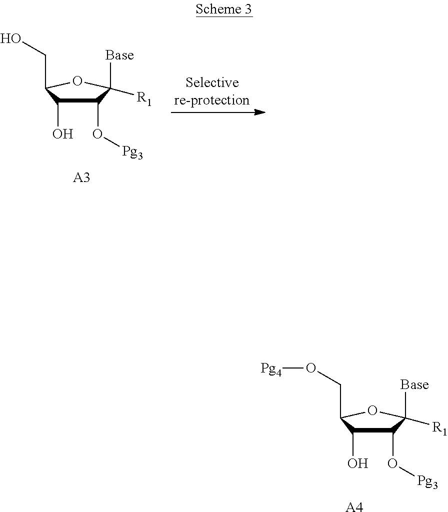

wherein Pg₄ is a protecting group; and
R₁, Pg₃, and a Base are as defined above.

### Step 4: Preparation of A5

Oxidizing compound A4 to obtain the C3′ ketone—compound A5 (as shown in scheme 4)

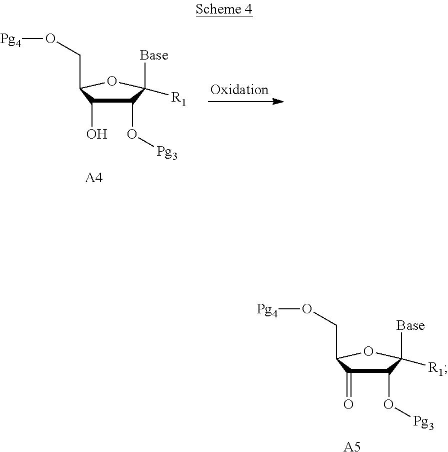

wherein Pg₄, R₁, Pg₃, and a Base are as defined above.

### Step 5: Preparation of A6

Halogenating Compound A5 to obtain the C3-dihalo compound A6 (as shown in scheme 5)

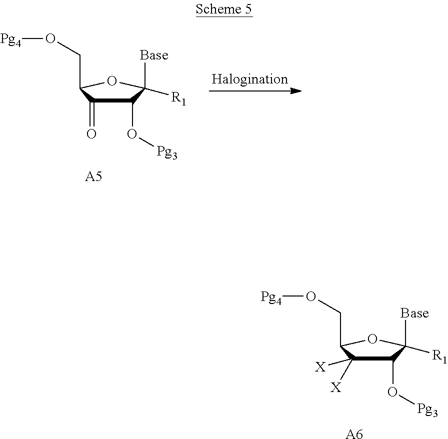

wherein X is a halo; and
wherein Pg₄, R₁, Pg₃, and a Base are as defined above.

### Step 6: Preparation of A7

selectively de-protecting C5′ of compound A6 to obtain compound A7 (as shown in scheme 6)

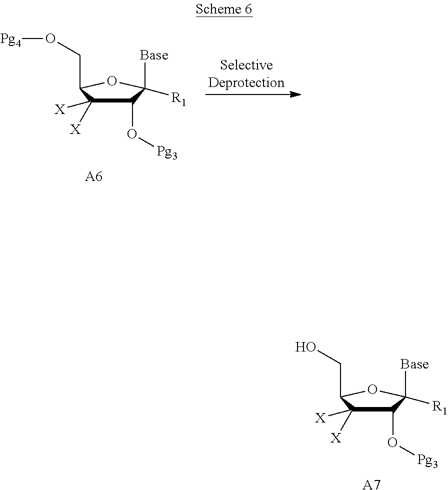

wherein X, Pg₄, R₁, Pg₃, and a Base are as defined above.

### Step 7: Preparation of A8

Oxidizing compound A7 followed by elimination and reduction to obtain compound A8 (as shown in scheme 7)

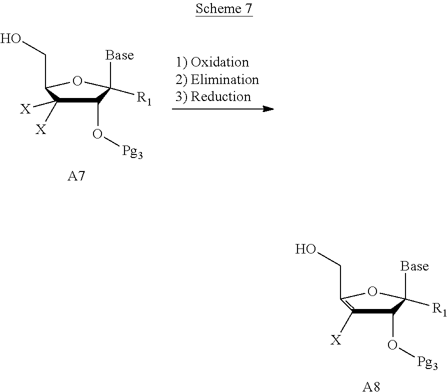

wherein X, R₁, Pg₃, and a Base are as defined above.

### Step 8: Preparation of Compound A16

Deprotection of compound A8 followed by substitution at position C5′ with R₇ group (as shown in scheme 8);

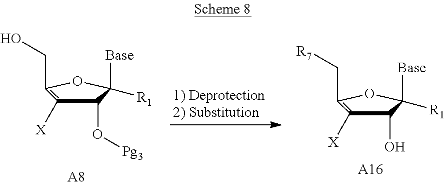

wherein X, R₁, Pg₃, and a Base are as defined above; and
R₇ is OH,

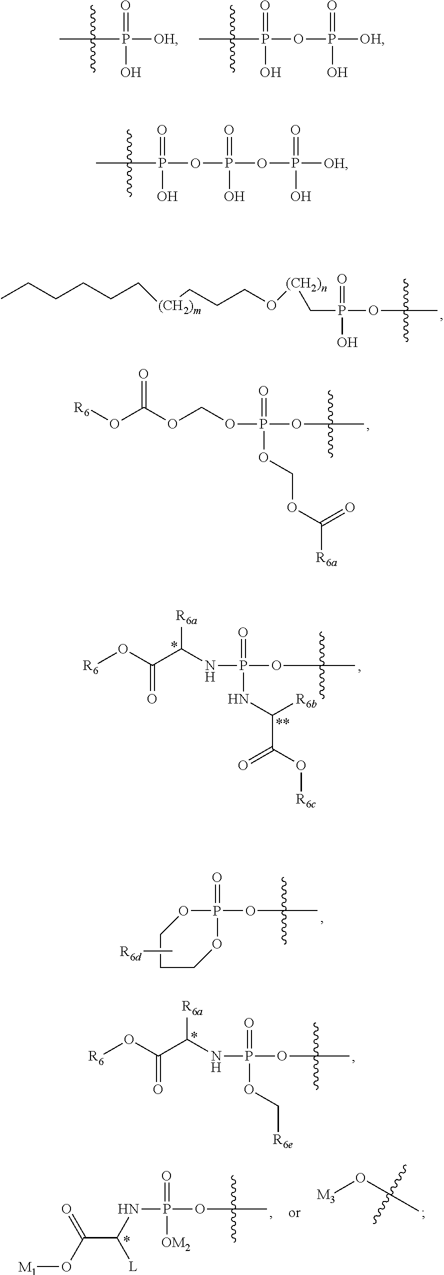

M₁ is a branched or linear alkyl;
L is a side chain of a natural or unnatural amino acid;
M₂ is substituted or unsubstituted aryl, or substituted or unsubstituted heteroaryl;
M₃ is

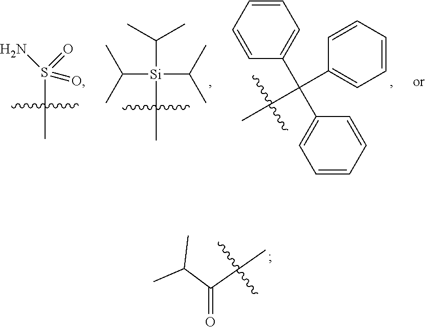

n is an integer between 1-4;
m is an integer between 1-4;
R₆, R₆ₐ, R_{6b}, and R_{6c} are each independently branched or linear alkyl;
R_{6d} is H or an alkyl; and
R₆ₑ is a haloalkyl.

In some embodiments, provided herein is a process for the preparation of compound A15, as represented by the structure compound A15

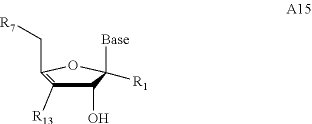

wherein
R₁ is H, —CN, —N₃, an alkyne-R₈, or an alkyl;
R₁₃ is a small alkyl (C1-C6);
R₇ is OH,

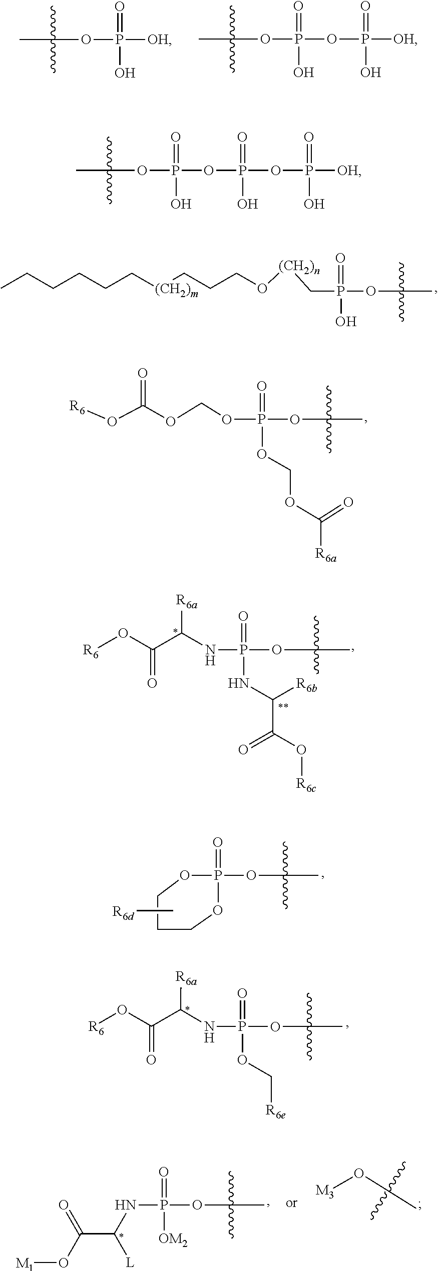

M₁ is a branched or linear alkyl;
L is a side chain of a natural or unnatural amino acid;
M₂ is substituted or unsubstituted aryl, or substituted or unsubstituted heteroaryl;
M₃ is

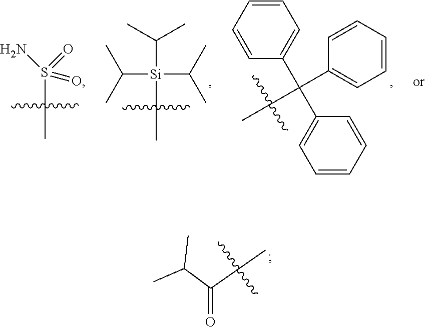

n is an integer between 1-4;
m is an integer between 1-4;
R₆, R₆ₐ, R_{6b}, and R_{6c} are each independently branched or linear alkyl;
R_{6d} is H or an alkyl; and
R₆ₑ is a haloalkyl.

Base is N-linked natural base, N-linked modified purine or pyrimidine base or C-linked base; wherein compound 15A is a compound of Formulas I-VI and X.

In some embodiments, provided herein is a process for the preparation of compound A14, wherein compound A14 is a compound of Formulas I-IV and X, and also an intermediate of a compound A15. In some embodiment, provided herein is a process for the preparation of compound A15,
wherein the process comprises:

### Step 1: Preparation of A9a and A9b

Reacting compound A5 with reagents such as Grignard reagents and the like to obtain compounds A9a and A9b (as shown in scheme 9)

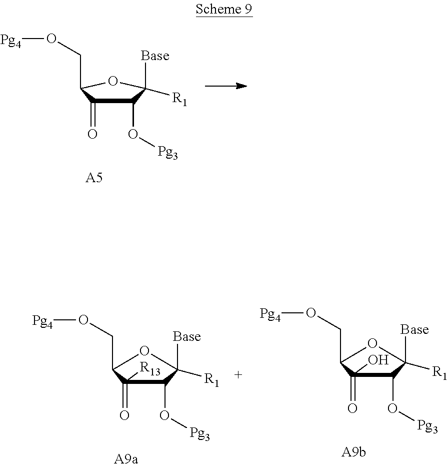

wherein
R₁, R₁₃, Pg₃, Pg₄, and a Base are as defined above.

### Step 2: Preparation of A10a-b

Converting the OH group into a leaving group (LG) to obtain compounds A10a-b
(as shown in scheme 10)

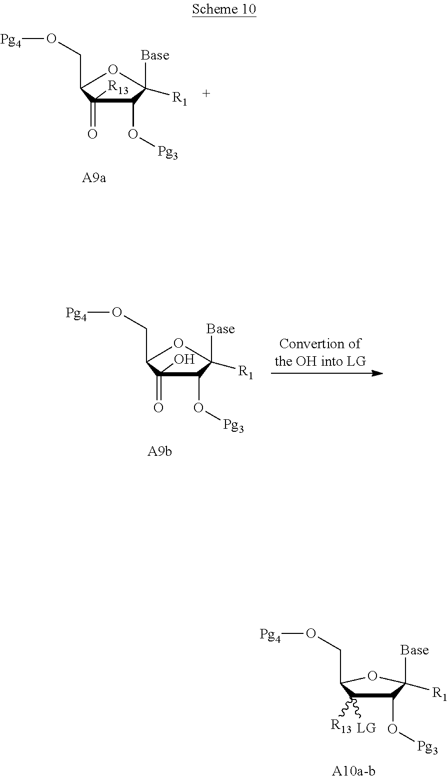

wherein
LG is leaving group; and
R₁, R₁₃, Pg₃, Pg₄, and a Base are as defined above.

### Step 3: Preparation of A11a-b

selectively de-protecting C5′ of compounds A10a-b to obtain compounds A11a-b
(as shown in scheme 11)

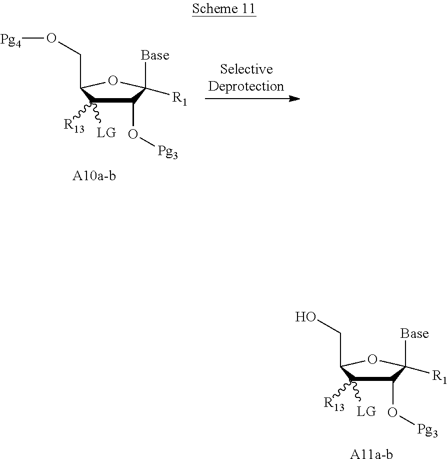

wherein
R₁, R₁₃, Pg₃, Pg₄, LG and a Base are as defined above.

### Step 4: Preparation of A13

Oxidizing compounds A11a-b followed by elimination and reduction to obtain compound A13 (as shown in scheme 12)

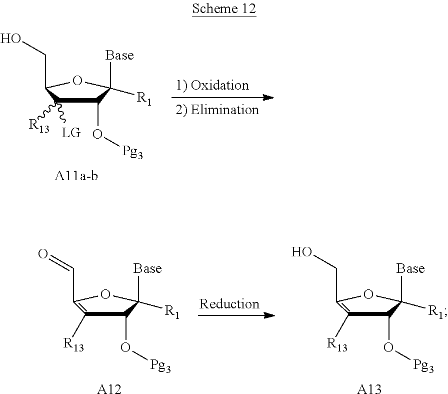

wherein
R₁, R₁₃, Pg₃, LG and a Base are as defined above.

### Step 5: Preparation of A14—Deprotection of A13

Removal of the protecting group of compound A13 (as shown in scheme 13)

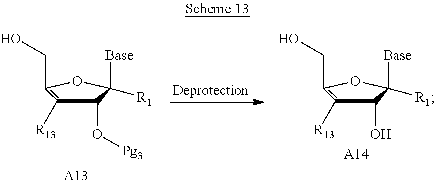

wherein
R₁, R, Pg₃, and a Base are as defined above.

In one embodiment, the base can be substituted with a protecting group. In one embodiment, the protecting group substituted on the base is removed at step 5.

### Step 6: Preparation of A15

Substitution of position C5′ with R₇ group (as shown in scheme 14)

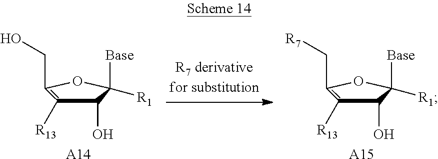

wherein
R₁, R₁₃, and the Base are as defined above;
R₇ is OH,

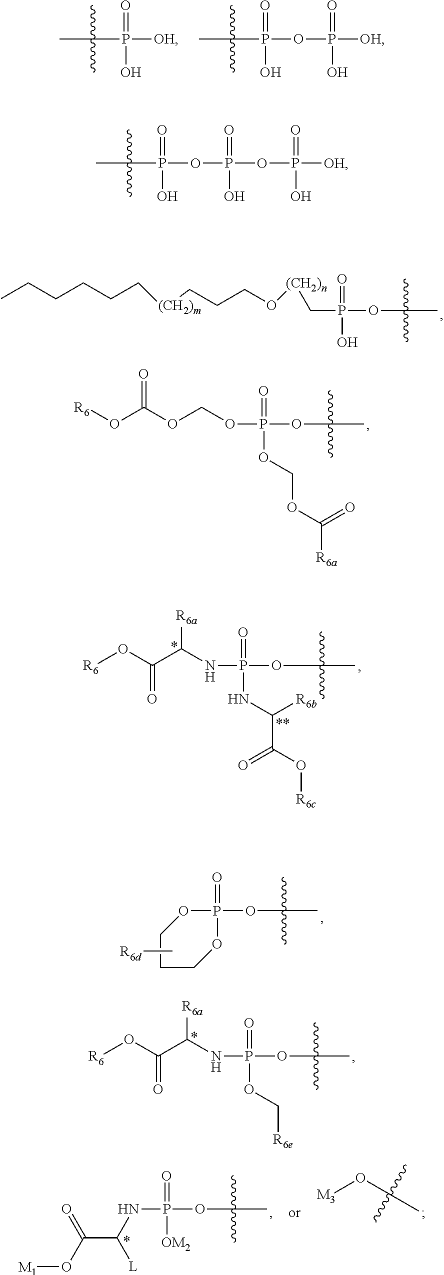

M₁ is a branched or linear alkyl;
L is a side chain of a natural or unnatural amino acid;
M₂ is substituted or unsubstituted aryl, or substituted or unsubstituted heteroaryl;
M₃ is

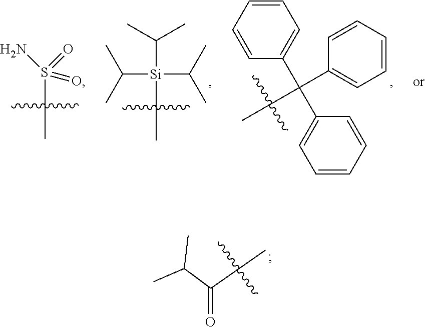

n is an integer between 1-4;
m is an integer between 1-4;
R₆, R₆ₐ, R_{6b}, and R_{6c} are each independently branched or linear alkyl;
R_{6d} is H or an alkyl; and
R₆ₑ is a haloalkyl.

In some embodiments, the base of Compounds A1-A16 is as defined in Formulas I-IX. In another embodiment, the base of Compounds A1-A14 is substituted with one or more protecting group.

In one embodiment, the reagent used in the process of scheme 1 for selective protection comprises silyl reagent such as

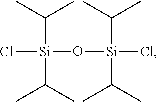 [SMILES: CC(C)[Si](Cl)(O[Si](Cl)(C(C)C)C(C)C)C(C)C]

### TsCl, MsCl,

In one embodiment, the reagent used in the process of scheme 1 for protection with an orthogonal protecting group comprises acyl chloride for example and not limiting to, benzoyl chloride, and acetyl chloride.

In one embodiment, the process of scheme 1 for protection with an orthogonal protecting group comprises a base. In another embodiment, the base is pyridine.

In one embodiment, the deprotection step of scheme 2 comprises reacting compound A2 with any known reagent for deprotection of alcohol protecting group as disclosed in Greene's. (for example, TBAF/THF, MeNH₂ and the like)

In one embodiment, the selective deprotection of position C5′ of compound A6 as shown in scheme 6. comprises reacting compound A6 with any known reagent for deprotection of alcohol protecting group as disclosed in Greene's (for example, DCA and the like)

In one embodiment, the deprotection step of scheme 8 comprises reacting compound A8 with any known reagent for deprotection of alcohol protecting group as disclosed in Greene's, (for example MeNH₂ and the like).

In one embodiment, the deprotection step of scheme 13 comprises reacting compound A13 with any known reagent for deprotection of alcohol protecting group as disclosed in Greene's, (for example MeNH₂ and the like) to obtain compound A14.

In one embodiment, the oxidizing agent used in scheme 4 comprises: Martin's reagent, Des-Martin, chromium reagents (Jone's reagents) and the like.

In one embodiment, the halogenation in scheme 5, is fluorination, chlorination, bromination or iodination, wherein the fluorination comprises reacting compound A5 with fluorinating reagents such as but limited to DAST furnishes the C3′-difluoro compound A6.

In one embodiment, the conversion of the C3′-OH of compounds A9a-b is converted directly to a leaving group—such as but not limiting to OTs, OMs, to obtain compounds A10a-b.

In one embodiment, the conversion of the C3′-OH of compounds A9a-b is converted indirectly into a leaving group such as but not limiting to a halo, to obtain compounds A10a-b.

### Pharmaceutical Compositions

In some embodiments provided herein is a pharmaceutical composition comprising any one of the compounds disclosed herein. In some embodiments, provided herein is a pharmaceutical composition comprising a pharmaceutically acceptable salt of any one of the compounds disclosed herein. In some embodiments, provided herein is a pharmaceutical composition comprising any one of the compounds disclosed herein and a pharmaceutically acceptable carrier. In some embodiments, a pharmaceutical composition comprises a preparation of one or more of the ddh- or deoxy-ddh-compounds, described herein with other chemical components, such as physiologically (pharmaceutically) suitable carriers and excipients.

In some embodiments provided herein is a pharmaceutical composition comprising at least two of the compounds disclosed herein. In some embodiments, provided herein is a pharmaceutical composition comprising a pharmaceutically acceptable salt of at least two of the compounds disclosed herein. In some embodiments, provided herein is a pharmaceutical composition comprising at least two of the compounds disclosed herein and a pharmaceutically acceptable carrier. In some embodiments, a pharmaceutical composition comprises a preparation of one or more of the ddh- or deoxy-ddh-compounds, described herein with other chemical components, such as physiologically (pharmaceutically) suitable carriers and excipients.

Pharmaceutically acceptable excipients and carriers are known to those skilled in the art, and have been amply described in a variety of publications, including, for example, A. Gennaro (1995) “Remington: The Science and Practice of Pharmacy”, 19th edition, Lippincott, Williams, & Wilkins Formulations. The purpose of a pharmaceutical composition is to facilitate administration of a compound to an organism. In certain embodiments, a pharmaceutical composition provides the pharmaceutical dosage form of a drug.

In some embodiments, provided herein is a pharmaceutical composition comprising a ddh- or deoxy ddh-analog compound represented by the structure of any one of the following Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A14, A15, A16, 14, 15, 16, or a combination thereof. In some embodiments, provided herein is a pharmaceutical composition comprising a ddh- or deoxy ddh-analog compound represented by the structure of any one of the following Formulas: I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A14, A15, A16, 14, 15, 16, or a pharmaceutically acceptable salt thereof. In some embodiments, provided herein is a pharmaceutical composition comprising a ddh- or deoxy ddh-analog compound or their pharmaceutical salt represented by the structure of any one of the following Formulas: I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, A14, A15, A16, 14, 15, 16, or a combination thereof.

In some embodiments, a pharmaceutical composition comprises a compound or a pharmaceutically acceptable salt thereof represented by the structure of Formula I. In some embodiments, a pharmaceutical composition comprises a compound or a pharmaceutically acceptable salt thereof represented by the structure of Formula I1. In some embodiments, a pharmaceutical composition comprises a compound or a pharmaceutically acceptable salt thereof represented by the structure of Formula I2. In some embodiments, a pharmaceutical composition comprises a compound or a pharmaceutically acceptable salt thereof represented by the structure of Formula I3. In some embodiments, a pharmaceutical composition comprises a compound or a pharmaceutically acceptable salt thereof represented by the structure of Formula I4. In some embodiments, a pharmaceutical composition comprises a compound represented by the structure of Formula I5. In some embodiments, a pharmaceutical composition comprises a compound or a pharmaceutical acceptable salt thereof represented by the structure of Formula I6. In some embodiments, a pharmaceutical composition comprises a compound or a pharmaceutically acceptable salt thereof represented by the structure of Formula II. In some embodiments, a pharmaceutical composition comprises a compound or a pharmaceutically acceptable salt thereof represented by the structure of Formula II1. In some embodiments, a pharmaceutical composition comprises a compound or a pharmaceutically acceptable salt thereof represented by the structure of Formula II2. In some embodiments, a pharmaceutical composition comprises a compound or a pharmaceutically acceptable salt thereof represented by the structure of Formula II3. In some embodiments, a pharmaceutical composition comprises a compound or a pharmaceutically acceptable salt thereof represented by the structure of Formula II4. In some embodiments, a pharmaceutical composition comprises a compound or a pharmaceutically acceptable salt thereof represented by the structure of Formula II5. In some embodiments, a pharmaceutical composition comprises a compound or a pharmaceutically acceptable salt thereof represented by the structure of Formula II6. In some embodiments, a pharmaceutical composition comprises a compound or a pharmaceutically acceptable salt thereof represented by the structure of Formula II7. In some embodiments, a pharmaceutical composition comprises a compound or a pharmaceutically acceptable salt thereof represented by the structure of Formula II8. In some embodiments, a pharmaceutical composition comprises a compound or a pharmaceutically acceptable salt thereof represented by the structure of Formula II9. In some embodiments, a pharmaceutical composition comprises a compound or a pharmaceutically acceptable salt thereof represented by the structure of Formula II10. In some embodiments, a pharmaceutical composition comprises a compound or a pharmaceutically acceptable salt thereof represented by the structure of Formula II11. In some embodiments, a pharmaceutical composition comprises a compound or a pharmaceutically acceptable salt thereof represented by the structure of Formula II12. In some embodiments, a pharmaceutical composition comprises a compound or a pharmaceutically acceptable salt thereof represented by the structure of Formula II13. In some embodiments, a pharmaceutical composition comprises a compound or a pharmaceutically acceptable salt thereof represented by the structure of Formula II14. In some embodiments, a pharmaceutical composition comprises a compound or a pharmaceutically acceptable salt thereof represented by the structure of Formula II15. In some embodiments, a pharmaceutical composition comprises a compound or a pharmaceutically acceptable salt thereof represented by the structure of Formula II16. In some embodiments, a pharmaceutical composition comprises a compound or a pharmaceutically acceptable salt thereof represented by the structure of Formula II17. In some embodiments, a pharmaceutical composition comprises a compound or a pharmaceutically acceptable salt thereof represented by the structure of Formula II18. In some embodiments, a pharmaceutical composition comprises a compound or a pharmaceutically acceptable salt thereof represented by the structure of Formula II19. In some embodiments, a pharmaceutical composition comprises a compound or a pharmaceutically acceptable salt thereof represented by the structure of Formula II20. In some embodiments, a pharmaceutical composition comprises a compound or a pharmaceutically acceptable salt thereof represented by the structure of Formula II21. In some embodiments, a pharmaceutical composition comprises a compound or a pharmaceutically acceptable salt thereof represented by the structure of Formula II22. In some embodiments, a pharmaceutical composition comprises a compound or a pharmaceutically acceptable salt thereof represented by the structure of Formula II23.

In some embodiments, a pharmaceutical composition comprises a compound or a pharmaceutically acceptable salt thereof represented by the structure of Formula III. In some embodiments, a pharmaceutical composition comprises a compound or a pharmaceutically acceptable salt thereof represented by the structure of Formula III1. In some embodiments, a pharmaceutical composition comprises a compound or a pharmaceutically acceptable salt thereof represented by the structure of Formula III2. In some embodiments, a pharmaceutical composition comprises a compound or a pharmaceutically acceptable salt thereof represented by the structure of Formula III3. In some embodiments, a pharmaceutical composition comprises a compound or a pharmaceutically acceptable salt thereof represented by the structure of Formula III4.

In some embodiments, a pharmaceutical composition comprises a compound or a pharmaceutically acceptable salt thereof represented by the structure of Formula IV. In some embodiments, a pharmaceutical composition comprises a compound or a pharmaceutically acceptable salt thereof represented by the structure of Formula IV1. In some embodiments, a pharmaceutical composition comprises a compound or a pharmaceutically acceptable salt thereof represented by the structure of Formula IV2. In some embodiments, a pharmaceutical composition comprises a compound or a pharmaceutically acceptable salt thereof represented by the structure of Formula IV3. In some embodiments, a pharmaceutical composition comprises a compound or a pharmaceutically acceptable salt thereof represented by the structure of Formula IV4. In some embodiments, a pharmaceutical composition comprises a compound or a pharmaceutically acceptable salt thereof represented by the structure of Formula IV5. In some embodiments, a pharmaceutical composition comprises a compound or a pharmaceutically acceptable salt thereof represented by the structure of Formula IV6.

In some embodiments, a pharmaceutical composition comprises a compound or a pharmaceutically acceptable salt thereof represented by the structure of Formula V. In some embodiments, a pharmaceutical composition comprises a compound or a pharmaceutically acceptable salt thereof represented by the structure of Formula V1. In some embodiments, a pharmaceutical composition comprises a compound or a pharmaceutically acceptable salt thereof represented by the structure of Formula V2. In some embodiments, a pharmaceutical composition comprises a compound or a pharmaceutically acceptable salt thereof represented by the structure of Formula V3. In some embodiments, a pharmaceutical composition comprises a compound or a pharmaceutically acceptable salt thereof represented by the structure of Formula V4. In some embodiments, a pharmaceutical composition comprises a compound or a pharmaceutically acceptable salt thereof represented by the structure of Formula V5. In some embodiments, a pharmaceutical composition comprises a compound or a pharmaceutically acceptable salt thereof represented by the structure of Formula V6. In some embodiments, a pharmaceutical composition comprises a compound or a pharmaceutically acceptable salt thereof represented by the structure of Formula V7. In some embodiments, a pharmaceutical composition comprises a compound or a pharmaceutically acceptable salt thereof represented by the structure of Formula V8. In some embodiments, a pharmaceutical composition comprises a compound or a pharmaceutically acceptable salt thereof represented by the structure of Formula V9. In some embodiments, a pharmaceutical composition comprises a compound or a pharmaceutically acceptable salt thereof represented by the structure of Formula V10. In some embodiments, a pharmaceutical composition comprises a compound or a pharmaceutically acceptable salt thereof represented by the structure of Formula V11. In some embodiments, a pharmaceutical composition comprises a compound or a pharmaceutically acceptable salt thereof represented by the structure of Formula V12. In some embodiments, a pharmaceutical composition comprises a compound or a pharmaceutically acceptable salt thereof represented by the structure of Formula V13. In some embodiments, a pharmaceutical composition comprises a compound or a pharmaceutically acceptable salt thereof represented by the structure of Formula V14. In some embodiments, a pharmaceutical composition comprises a compound or a pharmaceutically acceptable salt thereof represented by the structure of Formula V15. In some embodiments, a pharmaceutical composition comprises a compound or a pharmaceutically acceptable salt thereof represented by the structure of Formula V16. In some embodiments, a pharmaceutical composition comprises a compound or a pharmaceutically acceptable salt thereof represented by the structure of Formula V17. In some embodiments, a pharmaceutical composition comprises a compound or a pharmaceutically acceptable salt thereof represented by the structure of Formula V18. In some embodiments, a pharmaceutical composition comprises a compound or a pharmaceutically acceptable salt thereof represented by the structure of Formula V19. In some embodiments, a pharmaceutical composition comprises a compound or a pharmaceutically acceptable salt thereof represented by the structure of Formula V20. In some embodiments, a pharmaceutical composition comprises a compound or a pharmaceutically acceptable salt thereof represented by the structure of Formula V21. In some embodiments, a pharmaceutical composition comprises a compound or a pharmaceutically acceptable salt thereof represented by the structure of Formula V22. In some embodiments, a pharmaceutical composition comprises a compound or a pharmaceutically acceptable salt thereof represented by the structure of Formula V23. In some embodiments, a pharmaceutical composition comprises a compound or a pharmaceutically acceptable salt thereof represented by the structure of Formula VI. In some embodiments, a pharmaceutical composition comprises a compound or a pharmaceutically acceptable salt thereof represented by the structure of Formula VI1. In some embodiments, a pharmaceutical composition comprises a compound or a pharmaceutically acceptable salt thereof represented by the structure of Formula VI2. In some embodiments, a pharmaceutical composition comprises a compound or a pharmaceutically acceptable salt thereof represented by the structure of Formula VI3. In some embodiments, a pharmaceutical composition comprises a compound or a pharmaceutically acceptable salt thereof represented by the structure of Formula VI4. In some embodiments, a pharmaceutical composition comprises a compound or a pharmaceutically acceptable salt thereof represented by the structure of Formula VII. In some embodiments, a pharmaceutical composition comprises a compound or a pharmaceutically acceptable salt thereof represented by the structure of Formula VII1. In some embodiments, a pharmaceutical composition comprises a compound or a pharmaceutically acceptable salt thereof represented by the structure of Formula VII2. In some embodiments, a pharmaceutical composition comprises a compound or a pharmaceutically acceptable salt thereof represented by the structure of Formula VII3.

In some embodiments, a pharmaceutical composition comprises a compound or a pharmaceutically acceptable salt thereof represented by the structure of Formula VII4. In some embodiments, a pharmaceutical composition comprises a compound or a pharmaceutically acceptable salt thereof represented by the structure of Formula VII5. In some embodiments, a pharmaceutical composition comprises a compound or a pharmaceutically acceptable salt thereof represented by the structure of Formula VII6.

In some embodiments, a pharmaceutical composition comprises a compound or a pharmaceutically acceptable salt thereof represented by the structure of Formula VIII. In some embodiments, a pharmaceutical composition comprises a compound or a pharmaceutically acceptable salt thereof represented by the structure of Formula VIII1. In some embodiments, a pharmaceutical composition comprises a compound or a pharmaceutically acceptable salt thereof represented by the structure of Formula VIII2. In some embodiments, a pharmaceutical composition comprises a compound or a pharmaceutically acceptable salt thereof represented by the structure of Formula VIII3. In some embodiments, a pharmaceutical composition comprises a compound or a pharmaceutically acceptable salt thereof represented by the structure of Formula VIII4. In some embodiments, a pharmaceutical composition comprises a compound or a pharmaceutically acceptable salt thereof represented by the structure of Formula VIII5. In some embodiments, a pharmaceutical composition comprises a compound or a pharmaceutically acceptable salt thereof represented by the structure of Formula VIII6. In some embodiments, a pharmaceutical composition comprises a compound or a pharmaceutically acceptable salt thereof represented by the structure of Formula VIII7. In some embodiments, a pharmaceutical composition comprises a compound or a pharmaceutically acceptable salt thereof represented by the structure of Formula VIII8. In some embodiments, a pharmaceutical composition comprises a compound or a pharmaceutically acceptable salt thereof represented by the structure of Formula VIII9. In some embodiments, a pharmaceutical composition comprises a compound or a pharmaceutically acceptable salt thereof represented by the structure of Formula VIII10. In some embodiments, a pharmaceutical composition comprises a compound or a pharmaceutically acceptable salt thereof represented by the structure of Formula VIII11. In some embodiments, a pharmaceutical composition comprises a compound or a pharmaceutically acceptable salt thereof represented by the structure of Formula VIII12.

In some embodiments, a pharmaceutical composition comprises a compound or a pharmaceutically acceptable salt thereof represented by the structure of Formula VIII13. In some embodiments, a pharmaceutical composition comprises a compound or a pharmaceutically acceptable salt thereof represented by the structure of Formula VIII14. In some embodiments, a pharmaceutical composition comprises a compound or a pharmaceutically acceptable salt thereof represented by the structure of Formula VIII15. In some embodiments, a pharmaceutical composition comprises a compound or a pharmaceutically acceptable salt thereof represented by the structure of Formula VIII16. In some embodiments, a pharmaceutical composition comprises a compound or a pharmaceutically acceptable salt thereof represented by the structure of Formula VIII17. In some embodiments, a pharmaceutical composition comprises a compound or a pharmaceutically acceptable salt thereof represented by the structure of Formula VIII18. In some embodiments, a pharmaceutical composition comprises a compound or a pharmaceutically acceptable salt thereof represented by the structure of Formula VIII19. In some embodiments, a pharmaceutical composition comprises a compound or a pharmaceutically acceptable salt thereof represented by the structure of Formula VIII20. In some embodiments, a pharmaceutical composition comprises a compound or a pharmaceutically acceptable salt thereof represented by the structure of Formula VIII21. In some embodiments, a pharmaceutical composition comprises a compound or a pharmaceutically acceptable salt thereof represented by the structure of Formula VIII22. In some embodiments, a pharmaceutical composition comprises a compound or a pharmaceutically acceptable salt thereof represented by the structure of Formula VIII23.

In some embodiments, a pharmaceutical composition comprises a compound or a pharmaceutically acceptable salt thereof represented by the structure of Formula IX. In some embodiments, a pharmaceutical composition comprises a compound or a pharmaceutically acceptable salt thereof represented by the structure of Formula IX1. In some embodiments, a pharmaceutical composition comprises a compound or a pharmaceutically acceptable salt thereof represented by the structure of Formula IX2. In some embodiments, a pharmaceutical composition comprises a compound or a pharmaceutically acceptable salt thereof represented by the structure of Formula IX3. In some embodiments, a pharmaceutical composition comprises a compound or a pharmaceutically acceptable salt thereof represented by the structure of Formula IX4.

In some embodiments, a pharmaceutical composition comprises a compound or a pharmaceutically acceptable salt thereof represented by the structure of Formula X.

In some embodiments, a pharmaceutical composition comprises a compound or a pharmaceutically acceptable salt thereof represented by the structure of Formula X1.

In some embodiments, a pharmaceutical composition comprises a compound or a pharmaceutically acceptable salt thereof represented by the structure of Formula X2.

In some embodiments, a pharmaceutical composition comprises a compound or a pharmaceutically acceptable salt thereof represented by the structure of Formula X3

In some embodiments, a pharmaceutical composition comprises a compound or a pharmaceutically acceptable salt thereof represented by the structure of Formula X4.

In some embodiments, a pharmaceutical composition comprises a compound or a pharmaceutically acceptable salt thereof represented by the structure of Formula A14. In some embodiments, a pharmaceutical composition comprises a compound or a pharmaceutically acceptable salt thereof represented by the structure of Formula A15.

In some embodiments, a pharmaceutical composition comprises a compound or a pharmaceutically acceptable salt thereof represented by the structure of Formula A16.

In some embodiments, a pharmaceutical composition comprises a compound or a pharmaceutically acceptable salt thereof represented by the structure of Compound 14.

In some embodiments, a pharmaceutical composition comprises a compound or a pharmaceutically acceptable salt thereof represented by the structure of Compound 15.

In some embodiments, a pharmaceutical composition comprises a compound or a pharmaceutically acceptable salt thereof represented by the structure of Compound 16.

In one embodiment, provided herein is a pharmaceutical composition comprising a compound represented by the structure of Formula I, I1, I2, I3, I4, I5, I6, or combination thereof.

In one embodiment, provided herein is a pharmaceutical composition comprising a compound represented by the structure of Formula II, II1, II2, II3, II4, II5, II6, II7, II8, II9, II10, II11, II12, II13, II14, II15, II16, II17, II18, II19, II20, II21, II22, II23, or combination thereof.

In one embodiment, provided herein is a pharmaceutical composition comprising a compound represented by the structure of Formula III, III1, III2, III3, III4, or combination thereof.

In one embodiment, provided herein is a pharmaceutical composition comprising a compound represented by the structure of Formula IV, IV1, IV2, IV3, IV4, IV5, IV6, or combination thereof.

In one embodiment, provided herein is a pharmaceutical composition comprising a compound represented by the structure of Formula V, V1, V2, V3, V4, V5, V6, V7, V8, V9, V10, V11, V12, V13, V14, V15, V16, V17, V18, V19, V20, V21, V22, V23, or combination thereof.

In one embodiment, provided herein is a pharmaceutical composition comprising a compound represented by the structure of Formula VI, VI1, VI2, VI3, VI4, or combination thereof.

In one embodiment, provided herein is a pharmaceutical composition comprising a compound represented by the structure of Formula VII, VII1, VII2, VII3, VII4, VII5, VII6, or combination thereof.

In one embodiment, provided herein is a pharmaceutical composition comprising a compound represented by the structure of Formula VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, or combination thereof.

In one embodiment, provided herein is a pharmaceutical composition comprising a compound represented by the structure of Formula IX, IX1, IX2, IX3, IX4, or combination thereof.

In one embodiment, provided herein is a pharmaceutical composition comprising a compound represented by the structure of Formula X, X1, X2, X3, X4, or combination thereof.

In one embodiment, provided herein is a pharmaceutical composition comprising a compound represented by the structure of Formula A14, A15, A16, or combination thereof.

In one embodiment, provided herein is a pharmaceutical composition comprising a compound represented by the structure of Compound 14, 15, or combination thereof.

In one embodiment, provided herein is a pharmaceutical composition comprising a compound represented by the structure of Formula I, II, III, IV, V, VI, VI1, VIII, IX or X, or a combination thereof.

In one embodiment, provided herein is a pharmaceutical composition comprising at least two of the following Formulas: I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16.

In one embodiment, provided herein is a pharmaceutical composition comprising at least three of the following Formulas: I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16

In some embodiment, a pharmaceutical composition comprising a compound represented by the structure of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises a pharmaceutically acceptable carrier. In some embodiment, a pharmaceutical composition comprising a compound represented by the structure of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises a pharmaceutically acceptable excipient.

In some embodiment, a pharmaceutical composition comprising a compound represented by the structure of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, comprises a pharmaceutically acceptable carrier. In some embodiment, a pharmaceutical composition comprising a compound represented by the structure of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, comprises a pharmaceutically acceptable excipient.

In some embodiment, a pharmaceutical composition comprises at least two compounds represented by the structures of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16. In some embodiment, a pharmaceutical composition comprises at least two compounds represented by the structures of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof.

In some embodiment, a pharmaceutical composition comprises at least two compounds represented by the structures of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 and a pharmaceutically acceptable carrier. In some embodiment, a pharmaceutical composition comprises at least two compounds represented by the structures of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, and a pharmaceutically acceptable carrier.

In some embodiment, a pharmaceutical composition comprises at least two compounds represented by the structures of any one of Formulas I, I1, I2, I3, I4, I5, I6, II, II1, II2, II3, II4, I5, II6, II7, II8, II9, II10, II11, II12, II13, II14, I15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 and a pharmaceutically acceptable excipient. In some embodiment, a pharmaceutical composition comprises at least two compounds represented by the structures of any one of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, and a pharmaceutically acceptable excipient.

In one embodiment, the pharmaceutical composition comprises ddh- or deoxy-ddh-compounds that are in a prodrug form as described herein, comprising a protective chemical group.

In some embodiments, a pharmaceutical composition comprises any one of the compounds represented by the structures disclosed herein, and a pharmaceutically acceptable carrier. In some embodiments, a pharmaceutical composition comprises at least one of the compounds represented by the structures disclosed herein, and a pharmaceutically acceptable carrier. In some embodiments, a pharmaceutical composition comprises at least 2, 3, 4, 5, or 6 of the compounds represented by the structures disclosed herein, and a pharmaceutically acceptable carrier.

In some embodiments, a composition with an appropriate physiologically acceptable carrier may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients and/or suitable excipients such as salts, buffers and stabilizers may, but need not, be present within the composition. As used herein, the term “pharmaceutically acceptable carrier” may in some embodiments be used interchangeably with the terms “physiological carrier”, “physiologically acceptable carrier”, “pharmaceutically acceptable diluent” or “pharmaceutically acceptable excipient” having all the same qualities and meanings.

Administration of a pharmaceutical composition disclosed herein may be achieved by a variety of different routes, including oral, parenteral, nasal, intravenous, intradermal, subcutaneous or topical. In some embodiments, modes of administration depend upon the nature of the condition to be treated or prevented. In some embodiments, an amount that, following administration, reduces, inhibits, prevents or delays the progression and/or metastasis of a cancer is considered effective. In some embodiments, an amount that, following administration, reduces, inhibits, prevents or delays the progression of a viral infection or disease associated with a viral infection is considered effective. In some embodiments, an amount that, following administration, reduces, inhibits, prevents or delays the progression of a bacterial infection or disease associated with a bacterial infection is considered effective. In some embodiments, an amount that, following administration, reduces, inhibits, prevents or delays the progression of an immune disease or disorder is considered effective. In some embodiments, an amount that, following administration, reduces, inhibits, prevents or delays the progression of an autoimmune disease or disorder is considered effective. A skilled artisan would appreciate that the term “physiologically acceptable carrier, diluent or excipient”, may in some embodiments be used interchangeably with the term “pharmaceutically acceptable carrier” having all the same means and qualities.

A pharmaceutical composition may be in the form of a solid or liquid. In some embodiments, the pharmaceutically acceptable carrier(s) are particulate, so that the compositions are, for example, in tablet or powder form. The pharmaceutically acceptable carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible pharmaceutically acceptable carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid pharmaceutically acceptable carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of surfactants, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a ddh- or deoxy-ddh-compound as herein disclosed, such that a suitable dosage will be obtained.

The pharmaceutical composition may be intended for topical administration, in which case the pharmaceutically acceptable carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredient (a ddh- or deoxy-ddh-compound) may be encased in a gelatin capsule. The pharmaceutical composition in solid or liquid form may include an agent that binds to the ddh- or deoxy-ddh-compounds as disclosed herein, and thereby assists in the delivery of the compound. Suitable agents that may act in this capacity include monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a ddh- or deoxy-ddh-compound as described herein, and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the ddh- or deoxy-ddh-composition so as to facilitate dissolution or homogeneous suspension of ddh- or deoxy-ddh-compound in the aqueous delivery system.

The compositions may be administered in a therapeutically effective amount, which will vary depending upon a variety of factors including the activity of the ddh- or deoxy-ddh-compound employed; the metabolic stability and length of action of the ddh- or deoxy-ddh-compound; the age, body weight, general health, sex, and diet of the patient; the mode and time of administration; the rate of excretion; the drug combination; the severity of the particular allergic or respiratory disorder or condition; and the subject undergoing therapy.

In some embodiments, a pharmaceutically acceptable carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens, phenols or cresols, mercurials, chlorobutanol, methyl and propyl p-hydroxybenzoic acid esters, thimerosal, benzalkonium chloride and benzethonium chloride); antioxidants (such as ascorbic acid and sodium bisulfite; methionine, sodium thiosulfate, platinum, catalase, citric acid, cysteine, thioglycerol, thioglycolic acid, thiosorbitol, butylated hydroxyanisol, butylated hydroxytoluene, and/or propyl gallate) and chelating agents (such as ethylenediaminetetraacetic acid (EDTA)); buffers (such as acetates, citrates and phosphates). If administered intravenously, suitable pharmaceutically acceptable carriers include physiological saline or phosphate buffered saline (PBS), and solutions containing thickening and solubilizing agents, such as glucose, polyethylene glycol, polypropylene glycol and mixtures thereof.

The compositions comprising a ddh- or deoxy-ddh-compound as described herein, pharmaceutical salts thereof, may be prepared with pharmaceutically acceptable carriers that protect the ddh- or deoxy-ddh-compound against rapid elimination from the body, such as time release formulations or coatings. Such pharmaceutically acceptable carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

In some embodiments, the terms “pharmaceutical composition” and “composition” may be used interchangeably herein, having all the same meanings and qualities.

### Methods of Use

In some embodiments, the present disclosure provides compounds represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, for use in the treatment of a disease in a subject in need thereof. In some embodiments, the present disclosure provides a pharmaceutical composition comprising compounds represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, for use in the treatment of a disease in a subject in need thereof.

In some embodiments, the present disclosure provides a combination of compounds represented by the structure of I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, for use in the treatment of a disease in a subject in need thereof.

In some embodiments, the present disclosure provides a pharmaceutical composition comprising a combination of compounds represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, for use in the treatment of a disease in a subject in need thereof.

The ddh- or deoxy-ddh-compounds may in certain embodiments, be used to treat a disease as a result of a viral infection. In some embodiments, the ddh- or deoxy-ddh-compound represented by the structure of I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 as disclosed herein, comprises antiviral activity.

In some embodiments, the ddh- or deoxy-ddh-compound, represented by the structure of Formula I, I1, I2, I3, I4, I5, I6, II, II1, II2, II3, II4, I5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a DNA virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a RNA virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a double stranded (ds) DNA virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a single stranded (ss) DNA virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a (+) sense ssRNA virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a (−) sense ss RNA virus. In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a ssRNA-RT virus ((+) sense RNA with DNA intermediate in life-cycle). In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 comprises antiviral activity, wherein the virus is a dsDNA-RT virus.

In some embodiments, the ddh- or deoxy-ddh-compound, represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or pharmaceutical salts thereof, comprise a DNA and/or RNA chain terminating activity.

In some embodiments, treatment of a disease in a subject in need thereof, comprises treating a virus-induced disease, a cancer, an autoimmune disease, an immune disorder, a bacterial associated disease or infection, or a combination thereof. In some embodiments, treatment of a disease in a subject in need thereof, comprises treating a viral infection, a disease associated with a viral infection, or a cancer associated with a viral infection, or a combination thereof. In another embodiment, the disease is a virus-induced disease. In another embodiment, the disease is a cancer. In another embodiment, the cancer comprises a virus-associated cancer. In another embodiment, the disease is an autoimmune disease. In another embodiment, the disease is an immune disorder. In another embodiment, the disease is a bacterial associated disease. In another embodiment, the disease is a viral infection. In another embodiment, the disease is a disease associated with a viral infection. In another embodiment, the disease is a cancer associated with a viral infection.

In some embodiments, the viral infection comprises infection by an RNA virus. In some embodiments, the viral infection comprises infection by an RNA virus, wherein said RNA virus comprises a single stranded or double stranded virus. In some embodiments, the viral infection comprises infection by a DNA virus. In some embodiments, the viral infection comprises infection by an DNA virus, wherein said DNA virus comprises a single stranded or double stranded virus.

In another embodiment, methods of use treat a disease comprising an infection. In another embodiment, methods of use disclosed herein treat COVID19 because of SARS-CoV-2 infection.

In some embodiments, a method of treating a disease, treats a virus-induced disease. In some embodiments of a method of treating virus-induced disease, the disease is a Pneumoviridae virus infection. In some embodiments of a method of treating virus-induced disease, the disease is a Picornaviridae virus infection. In some embodiments of a method of treating virus-induced disease, the disease is a Flaviviridae virus infection. In some embodiments of a method of treating virus-induced disease, the disease is a Filoviridae virus infection.

In some embodiments, said Pneumoviridae virus infection comprises a respiratory syncytial virus infection or a human metapneumovirus infection. In some embodiments, said Pneumoviridae virus infection comprises a respiratory syncytial virus infection. In some embodiments, said Pneumoviridae virus infection comprises a human metapneumovirus infection. In some embodiments, said Picornaviridae virus infection comprises a human rhinovirus infection. In some embodiments, a Flaviviridae virus infection comprises a dengue virus infection, a yellow fever virus infection, a West Nile virus infection, a zika virus infection, or a hepatitis C virus infection. In some embodiments, a Flaviviridae virus infection comprises a dengue virus infection. In some embodiments, a Flaviviridae virus infection comprises a yellow fever virus infection. In some embodiments, a Flaviviridae virus infection comprises a West Nile virus infection. In some embodiments, a Flaviviridae virus infection comprises a zika virus infection. In some embodiments, a Flaviviridae virus infection comprises a hepatitis C virus infection. In some embodiments, a Filoviridae virus infection comprises an Ebola virus infection.

Subjects infected with EBV have an increased the risk for the development of several cancers and autoimmune diseases. Diseases associate with EBV infection included but are not limited to infectious mononucleosis, hemophagocytic lymphohistiocytosis, non-malignant or premalignant or malignant lymphoproliferative diseases such as Burkitt lymphoma, Hodgkin's lymphoma, non-lymphoid malignancies such as gastric cancer and nasopharyngeal carcinoma, hairy leukoplakia, central nervous system lymphomas, and multiple sclerosis. (See, Drosu et al., (2020) Tenofovir prodrugs potently inhibit Epstein-Barr virus lytic DNA replication by targeting the viral DNA polymerase. PNAS 117 (22): 12368-12374.) In some embodiment, methods of use disclosed herein treat an EBV infection-associated disease comprising infectious mononucleosis, hemophagocytic lymphohistiocytosis, non-malignant or premalignant or malignant lymphoproliferative diseases such as Burkitt lymphoma, Hodgkin's lymphoma, non-lymphoid malignancies such as gastric cancer and nasopharyngeal carcinoma, hairy leukoplakia, central nervous system lymphomas, and multiple sclerosis.

Double-stranded (ds) DNA virus infections often occur concomitantly in immunocompromised patients. In another embodiment, methods of use disclosed herein treat a viral induced disease occurring in an immunocompromised or immunosuppressed subject. In some embodiments, methods of use treat an immunocompromised patient infected with a BK virus, an adenovirus, a herpesvirus including Epstein-Barr virus, a poxvirus, or a polyoma virus including BK virus or JC virus (human polyomavirus 2)). Patients undergoing solid organ transplantation or allogeneic hematopoietic cell transplant (allo-HCT) are susceptible to viral infection, due to immunosuppressive environment created to support the transplant. In some embodiments, these patients are suffering from diseases including but not limited to nephropathy or hemorrhagic cystitis, etc. These transplant patients are particularly susceptible to dsDNA viral infections, for example EBV infections or polyoma viral infection including BKV and JCV infections.

In some embodiments, methods of treating disclosed herein, treat a disease associated with a dsDNA viral infection. In some embodiments, diseases associated dsDNA viral infections include diseases associated with a hematopoietic cell transplantation including nephropathy, hemorrhagic cystitis, etc.

In some embodiments, methods of use treat an immunosuppressed transplant patient, for example a subject undergoing a solid organ transplantation or a hematopoietic cell transplantation, wherein said patient has a dsDNA viral infection. In some embodiments, methods of use treat an immunosuppressed transplant patient, for example a subject undergoing a solid organ transplantation or a hematopoietic cell transplantation, wherein said patient has an EBV, BKV, herpes virus-6, adenovirus, CMV, or JCV infection. (See for example, Chemaly et al., (2019) In vitro comparison of currently available and investigational antiviral agents against pathogenic human double-stranded DNA viruses: A systematic literature review. Antiviral Research 163: 50-58.) In some embodiments, methods of use treat an immunosuppressed transplant patient with an EBV infection. In some embodiments, methods of use treat an immunosuppressed transplant patient with an BKV infection. In some embodiments, methods of use treat an immunosuppressed transplant patient with a herpes virus-6 infection. In some embodiments, methods of use treat an immunosuppressed transplant patient with an adenovirus infection. In some embodiments, methods of use treat an immunosuppressed transplant patient with a CMV infection. In some embodiments, methods of use treat an immunosuppressed transplant patient with a JCV infection.

In another embodiment, a disease treated by methods disclosed herein is caused by a viral infection, wherein the virus is selected from the group consisting of norovirus, rotavirus, hepatitis virus A, B, C, D, or E, rabies virus, West Nile virus, enterovirus, echovirus, coxsackievirus, herpes simplex virus (HSV), varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus (MERS-CoV), SARS coronavirus 2 (SARS-CoV-2), Epstein-Barr virus (EBV), influenza virus, influenza virus A2, influenza virus B, influenza virus A (H1N1), respiratory syncytial virus (RSV), polyoma viruses, JC virus, BK virus, Tacaribe virus, Ebola virus, Dengue virus, Pneumoviridae virus, respiratory syncytial virus, human metapneumovirus, Picornaviridae virus, human rhinovirus, Flaviviridae virus, yellow fever virus, hepatitis C virus (HCV), and Filoviridae virus, and any combination thereof.

In another embodiment, the disease is caused by an EBV infection. In another embodiment, the disease is caused by an BKV infection. In another embodiment, the disease is caused by a SARS-CoV-2 infection.

In another embodiment, the disease is caused by a virus selected from the group consisting of norovirus, rotavirus, hepatitis virus A, B, C, D, or E, rabies virus, West Nile virus, enterovirus, echovirus, coxsackievirus, herpes simplex virus (HSV), varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus (MERS-CoV), SARS coronavirus 2 (SARS-CoV-2), Epstein-Barr virus (EBV), influenza virus, influenza virus A2, influenza virus B, influenza virus A (H1N1), respiratory syncytial virus (RSV), polyoma viruses, JC virus, BK virus, Tacaribe virus, Ebola virus, Dengue virus, Pneumoviridae virus, respiratory syncytial virus, human metapneumovirus, Picornaviridae virus, human rhinovirus, Flaviviridae virus, yellow fever virus, hepatitis C virus (HCV), and Filoviridae virus, and any combination thereof.

In some embodiments, the methods of treating comprising use of a ddh- or deoxy-ddh-compound, a pharmaceutically acceptable salt thereof, as disclosed herein, terminates polynucleotide chain synthesis in a cell. In some embodiments, methods of use of a ddh- or deoxy-ddh-compound inhibit or reduce viral polynucleotide chain synthesis. In some embodiments, methods of use of a ddh- or deoxy-ddh-compound, treat a subject in need suffering from a viral infection or suffering from a disease associated with a viral infection, included a cancer associated with a viral infection, by inhibiting or reducing viral polynucleotide chain synthesis.

In one embodiment, a viral infection is caused by viruses in the Baltimore classification Group I group of viruses: double-stranded DNA viruses (e.g., Adenoviruses, Herpesviruses including Epstein-Barr virus, Poxviruses, Polyoma viruses including BK virus and JC virus (human polyomavirus 2)). In another embodiment, the viral infection is caused by viruses in the Baltimore classification Group II group of viruses: single-stranded (or “sense”) DNA viruses (e.g., Parvoviruses). In another embodiment, the viral infection is caused by viruses in the Baltimore classification Group III group of viruses: double-stranded RNA viruses (e.g., Reoviruses). In another embodiment, the viral infection is caused by viruses in the Baltimore classification Group IV group of viruses: single-stranded (sense) RNA viruses (e.g., Picornaviruses, Togaviruses, Coronavirus including SARS-CoV-2). In another embodiment, the viral infection is caused by viruses in the Baltimore classification Group V of viruses: single-stranded (antisense) RNA viruses (e.g., Orthomyxoviruses, Rhabdoviruses). In another embodiment, the viral infection is caused by viruses in the Baltimore classification Group VI group of viruses: single-stranded (sense) RNA viruses with DNA intermediate in life-cycle (e.g., Retroviruses). In another embodiment, the viral infection is caused by viruses in the Baltimore classification Group VII group of viruses: double-stranded DNA viruses with RNA intermediate in life-cycle (e.g., Hepadnaviruses).

In one embodiment, the virus-induced disease can be respiratory viral infection (e.g., common cold, seasonal influenzas), gastrointestinal viral infection, liver viral infection, nervous system viral infection, skin viral infection, sexually transmitted viral infection, placental viral infection, or fetal viral infection.

In one embodiment, examples of viral induced disease include, but are not limited to, gastroenteritis, keratoconjunctivitis, pharyngitis, croup, pharyngoconjunctival fever, pneumonia, cystitis (Adenovirus), Hand, foot and mouth disease, pleurodynia, aseptic meningitis, pericarditis, myocarditis (Coxsackievirus), infectious mononucleosis, Burkitt's lymphoma, Hodgkin's lymphoma, nasopharyngeal carcinoma (Epstein-Barr virus), acute hepatitis, chronic hepatitis, hepatic cirrhosis, hepatocellular carcinoma, herpes labialis, cold sores, gingivostomatitis in children, tonsillitis & pharyngitis in adults, skin vesicles, mucosal ulcers, oral and/or genital ulcers, Aseptic meningitis (Herpes simplex virus, type 2), Cytomegalic inclusion disease, liver, lung and spleen diseases in the newborn, congenital seizures in the newborn (Cytomegalovirus), Kaposi sarcoma, multicentric Castleman disease, primary effusion lymphoma (Human herpesvirus, type 8), AIDS (HIV), influenza, Reye syndrome (Influenza virus), measles, postinfectious encephalomyelitis (Measles virus), mumps, hyperplastic epithelial lesions (common, flat, plantar and anogenital warts, laryngeal papillomas, epidermodysplasia verruciformis), cervical carcinoma, squamous cell carcinomas (Human papillomavirus), bronchiolitis, common cold (Parainfluenza virus), poliomyelitis (Poliovirus), rabies, influenza-like syndrome, severe bronchiolitis with pneumonia (Respiratory syncytial virus), congenital rubella, German measles (Rubella virus), chickenpox, herpes zoster, Congenital varicella syndrome (Varicella-zoster virus).

In one embodiment, the disease is caused by one or more of the following viruses: norovirus, rotavirus, hepatitis virus A, B, C, D, or E, rabies virus, West Nile virus, enterovirus, echovirus, coxsackievirus, herpes simplex virus (HSV), varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus (MERS-CoV), SARS coronavirus 2 (SARS-CoV-2), Epstein-Barr virus (EBV), influenza virus, influenza virus A2, influenza virus B, influenza virus A (H1N1), respiratory syncytial virus (RSV), polyoma viruses, JV virus, BK virus, Tacaribe virus, Ebola virus, Pneumoviridae virus, respiratory syncytial virus, human metapneumovirus, Picornaviridae virus, human rhinovirus, Flaviviridae virus, yellow fever virus, hepatitis C virus (HCV), and Filoviridae virus, and Dengue virus.

In one embodiment, the disease is COVID19 caused by SARS coronavirus 2. In one embodiment, the disease is the result of an EBV infection. In one embodiment, the disease is the result of an BKV infection.

In one embodiment, the viral infection is caused by viruses of human or non-human origin. In some embodiments, the viral infection is caused by modified or unmodified viruses that originate from animals or any foreign organism, for example, infection caused by SARS coronavirus, SARS-CoV-2, etc.

In some embodiments, treating a viral infection comprises protecting an organism from foreign nucleic acid invasion. In some embodiments, treating a viral infection comprises decreasing viral nucleic acid replication.

In one embodiment, the above-described composition comprising one or more ddh- or deoxy-ddh-compounds synthesized using methods known in the art, can be used in the treatment of cancer or a tumor.

Representative examples of cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In some embodiments, a virus comprises a bacteriophage. Bacteriophage may have a positive or negative impact on bacteria present in our microbiomes. Further, there is evidence that manipulating the microbiome may positively affect anti-cancer therapy in general and immune-based anti-cancer therapy. In some embodiments, methods of use of a ddh- or deoxy-ddh-compound disclosed herein inhibits or reduces bacteriophage polynucleotide chain synthesis. In some embodiments, methods of use of a ddh- or deoxy-ddh-compound disclosed herein for treating cancer, comprise improving the anti-cancer treatment or therapy. In some embodiments, methods of use of a ddh- or deoxy-ddh-compound disclosed herein target the microbiome. In some embodiments, the microbiome comprises a GI microbiome.

In some embodiments, methods of use of a ddh- or deoxy-ddh-compound disclosed herein inhibit or reduce bacteriophage polynucleotide chain synthesis. In some embodiments, methods of use of a ddh- or deoxy-ddh compound, treat a subject in need suffering from an imbalance of their microbiome, included an imbalance associate with a cancer.

In one embodiment, the above-described composition comprising one or more ddh or deoxy-ddh compounds synthesized using methods known in the art, can be used in the treatment of autoimmune disease. Representative examples of autoimmune disease include, but are not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenic purpura, type 1 diabetes, ulcerative colitis, uveitis, vasculitis, and vitiligo.

As it is generally known in the art, in order to function as DNA or RNA chain terminators in vivo, a ddh- or deoxy ddh compound would have to be converted by one or more cellular kinases into their 5′-triphosphate form before they can compete with the natural substrates (dNTPs for DNA synthesis and NTPs for RNA synthesis) in the DNA or RNA polymerization reaction. Thus, the “active metabolite” for the purpose of DNA or RNA chain termination is the ddh- or deoxy ddh compound in a 5′-triphosphate form. In other words, the products synthesized, for example by chemical synthesis, comprise active metabolites as DNA or RNA chain terminators, and these products or active metabolites are in a 5′-triphosphate form. However, in order to administer these products or active metabolites to the cells and allow transport across cell membrane, these products or active metabolites need to be administered in a form without an attached triphosphate, or as provided herein in a prodrug form.

In another embodiment, terminating polynucleotide chain synthesis increases termination of DNA chain synthesis, or increases termination of RNA chain synthesis, or a combination thereof. In another embodiment, terminating polynucleotide chain synthesis increases termination of DNA chain synthesis. In another embodiment, the terminating polynucleotide chain synthesis increases termination of RNA chain synthesis.

In certain embodiments, disclosed herein is a method of treating a disease in a subject in need thereof, comprising administering a therapeutically effective amount of a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, wherein said disease comprises a viral infection, a disease associated with a viral infection, or a cancer associated with a viral infection, or a combination thereof. In certain embodiments, disclosed herein is a method of treating a disease in a subject in need thereof, comprising administering a therapeutically effective amount of a pharmaceutical composition comprising a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, wherein said disease comprises a viral infection, a disease associated with a viral infection, or a cancer associated with a viral infection, or a combination thereof. In some embodiments, the virus comprises an oncovirus.

In one embodiment, a prodrug comprises a chemical structure that can be oxidized, reduced, aminated, deaminated, esterified, de-esterified, alkylated, dealkylated, acylated, deacylated, phosphorylated, dephosphorylated, photolyzed, hydrolyzed, or other functional group change or conversion resulting in the ability to be transported across the cell membrane.

In some embodiments provided herein is a ddh- or deoxy-ddh compound, for use in a method disclosed herein, wherein the compound is represented by Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16.

In one embodiment, pharmaceutical compositions used in methods disclosed herein comprising one or more ddh or deoxy-ddh compounds, can be provided to the subject with additional active agents to achieve an improved therapeutic effect as compared to treatment with each agent by itself. In another embodiment, additional active agents comprise an anti-viral agent, an anti-bacterial agent, or anti-cancer agent. In another embodiment, additional active agents comprise an antibiotic.

### Administration

Administration of a ddh- or deoxy ddh-compound or a pharmaceutically acceptable salt thereof, or a pharmaceutical composition comprising a ddh- or deoxy ddh-compound or a pharmaceutically acceptable salt thereof, as disclosed herein, may be achieved by a variety of different routes, including oral, parenteral, nasal, intravenous, intradermal, subcutaneous or topical. In some embodiments, modes of administration depend upon the nature of the condition to be treated or prevented. In some embodiments, an amount that, following administration, reduces, inhibits, prevents or delays the progression and/or metastasis of a cancer is considered effective. In some embodiments, an amount that, following administration, reduces, inhibits, prevents or delays the progression of a viral infection or disease associated with a viral infection is considered effective. In some embodiments, an amount that, following administration, reduces, inhibits, prevents or delays the progression of a bacterial infection or disease associated with a bacterial infection is considered effective. In some embodiments, an amount that, following administration, reduces, inhibits, prevents or delays the progression of an immune disease or disorder is considered effective. In some embodiments, an amount that, following administration, reduces, inhibits, prevents or delays the progression of an autoimmune disease or disorder is considered effective. An advantage of the compounds of this invention is that they are orally bioavailable and can be dosed orally.

Bacterial infections can be caused by numerous bacterial pathogens. In general, bacterial pathogens may be classified as either Gram-positive or Gram-negative pathogens. In some embodiments, the ddh or deoxy-ddh compounds described herein may comprise effective activity against either a Gram-positive bacterium or a Gram-negative bacterium, or both. In some embodiments, the ddh or deoxy-ddh compounds described herein comprise a broad-spectrum antibiotic activity. For example, but not limited to, in some embodiments, a bacterial infection may be the result of infection from a Streptococcus pneumoniae; Staphylococcus aureus; Haemophilus influenza, Myoplasma species, or Moraxella catarrhalis.

In certain embodiments, methods disclosed herein administer a ddh- or deoxy-ddh compound or a pharmaceutically acceptable salt thereof, for treating a disease in a subject. In some embodiments, an effective amount of a ddh- or deoxy-ddh compound or a pharmaceutically acceptable salt thereof or a composition thereof, is administered to a subject in need for treating a viral infection, a disease associated with a viral infection, or a cancer associated with a viral infection, or a combination thereof.

In some embodiments, administration of a ddh- or deoxy-ddh compound or a pharmaceutically acceptable salt thereof, or a composition thereof, comprises an agent capable of polynucleotide chain termination for viral RNA dependent polymerases. In some embodiments, administration of a ddh- or deoxy-ddh compound or a pharmaceutically acceptable salt thereof, or a composition thereof, comprises an agent capable of polynucleotide chain termination for viral RNA dependent polymerases, wherein said viral RNA comprises single stranded or double stranded RNA. In some embodiments, administration of a ddh- or deoxy-ddh compound or a pharmaceutically acceptable salt thereof, or a composition thereof, comprises an agent capable of polynucleotide chain termination for viral DNA dependent polymerases, wherein said viral DNA comprises single or double stranded DNA. In some embodiments, administration of a ddh- or deoxy-ddh compound or a pharmaceutically acceptable salt thereof, or a composition thereof, comprises an agent capable of functioning as an anti-viral chain termination. In some embodiments, administration of a ddh- or deoxy-ddh compound or a pharmaceutically acceptable salt thereof, or a composition thereof, comprises an agent capable of functioning as an anti-bacterial chain termination.

In another embodiment, terminating polynucleotide chain synthesis confers viral resistance to a cell.

In another embodiment, the cell is a eukaryotic cell. In another embodiment, said eukaryotic cell is a tumor cell, or is infected by a virus or a foreign DNA. In another embodiment, said eukaryotic cell is a tumor cell. In another embodiment, said eukaryotic cell is infected by a virus or a foreign DNA.

In some embodiments, the cell in which termination of polynucleotide chain synthesis is desired is a eukaryotic cell. In some embodiments, the eukaryotic cell is a tumor cell. In some embodiments, termination of polynucleotide chain synthesis decreases DNA replication in a cell. In some embodiments, termination of polynucleotide chain synthesis decreases RNA transcription in a cell.

In one embodiment, the present disclosure provides a method of terminating polynucleotide chain synthesis in a cell, the method comprises contacting the cell with a composition comprising one or more ddh or deoxy-ddh compounds or pharmaceutical salts thereof. In one embodiment, the present disclosure provides a method of terminating polynucleotide chain synthesis in a cell, the method comprises contacting the cell with a composition comprising one or more ddh or deoxy-ddh compounds or pharmaceutical salts thereof, comprising a protective chemical group.

In some embodiments, termination of polynucleotide chain synthesis comprises increased termination of DNA chain synthesis. In some embodiments, termination of polynucleotide chain synthesis comprises increased termination of RNA chain synthesis. In some embodiments, termination of polynucleotide chain synthesis decreases proliferation of a cell. In some embodiments, termination of polynucleotide chain synthesis comprises an anti-tumor activity.

In some embodiments, terminating polynucleotide chain synthesis in a cell comprises reducing polynucleotide chain synthesis in a cell by at least 1%, by at least 2%, by at least 3%, by at least 4%, by at least 5%, by at least 6%, by at least 7%, by at least 8%, by at least 9%, by at least 10%, by at least 20%, by at least 30%, by at least 40%, by at least 50%, by at least 60%, by at least 70%, by at least 80%, by at least 90%, or by 100%.

In some embodiments, terminating polynucleotide chain synthesis in a cell comprises reducing viral DNA replication. In some embodiments, terminating polynucleotide chain synthesis in a cell comprises reducing viral RNA chain synthesis. In some embodiments, terminating polynucleotide chain synthesis in a cell comprises reducing viral DNA or RNA chain synthesis without modifying DNA replication of the host cell.

In some embodiments, terminating polynucleotide chain synthesis in a cell comprises reducing eukaryotic DNA replication. In some embodiments, the eukaryotic cell is a tumor cell.

In some embodiments, terminating polynucleotide chain synthesis in a cell comprises reducing polynucleotide chain synthesis in a cell by between about 0% and about 10%, between about 10% and about 20%, between about 20% and about 30%, between about 30% and about 40%, between about 40% and about 50%, between about 50% and about 60%, between about 60% and about 70%, between about 70% and about 80%, between about 80% and about 90%, or between about 90% and about 100%.

In some embodiments, provided herein is a method for treating a disease wherein the method comprises administration of a pharmaceutical composition described herein.

In some embodiments, provided herein is a method for treating a disease wherein the method comprises administration of a compound or a pharmaceutically acceptable salt thereof, described herein.

In some embodiments, administration of a ddh- or deoxy-ddh compound or a pharmaceutically acceptable salt thereof, or a composition thereof, comprises an agent capable of treating viral infection. In some embodiments, administration of a ddh- or deoxy-ddh compound or a pharmaceutically acceptable salt thereof, or a composition thereof, comprises an agent capable of treating viral infection, wherein said virus comprises an RNA or a DNA virus. In some embodiments, administration of a ddh- or deoxy-ddh compound or a pharmaceutically acceptable salt thereof, or a composition thereof, comprises an agent capable of treating viral infection, wherein said virus comprises a single stranded or double stranded virus. In some embodiments, administration of a ddh- or deoxy-ddh compound or a pharmaceutically acceptable salt thereof, or a composition thereof, comprises an agent capable of treating a viral infection, wherein said virus comprises an oncovirus.

In some embodiments, the agent administered comprises a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a composition thereof. In some embodiments, the agent administered comprises a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IVI, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VIII, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, or a composition thereof. In some embodiments, the agent administered comprises a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a composition thereof. In some embodiments, the agent administered comprises a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, or a composition thereof.

In some embodiments, an at least one additional agent is administered in combination with a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a composition thereof.

In some embodiments, the additional agent is administered in the same composition as a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof. In some embodiments, the additional agent is administered in a different composition as a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof.

In some embodiments, the additional agent is administered concurrent with administration of a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof.

In some embodiments, the additional agent is administered prior to administration of a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof. In some embodiments, the additional agent is administered following administration of a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof.

In some embodiments, the additional agent is administered independent of administration of a compound of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof.

In some embodiments, an amount of a ddh- or deoxy-ddh compound disclosed herein, or a pharmaceutically acceptable salt thereof, or a composition thereof, that following administration treats a disease in a subject in need, is considered an effective amount. In some embodiments, an amount of a ddh- or deoxy-ddh compound or a pharmaceutically acceptable salt thereof or a composition thereof, that following administration treats a viral infection, a disease associated with a viral infection, or a cancer associated with a viral infection in a subject in need, is considered an effective amount.

In some embodiments, an amount of a compound of represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, that following administration treats a disease in a subject in need is considered an effective amount.

In some embodiments, an amount of a compound of represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, that following administration treats a viral infection, a disease associated with a viral infection, or a cancer associated with a viral infection in a subject in need, is considered an effective amount.

A skilled artisan would recognize that an “effective amount” (or, a “therapeutically effective amount”) may encompass an amount sufficient to affect a beneficial or desired clinical result upon treatment, for example but not limited to treating a disease in a subject, for example but not limited to a viral infection, a disease associated with a viral infection, or a cancer associated with a viral infection, in the subject in need.

Compounds represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, as disclosed herein in detail, or pharmaceutical compositions thereof, may be administered in a therapeutically effective amount (dose).

Determination of a therapeutically effective amount is well within the capability of those skilled in the art. For any preparation used in the methods disclosed herein, the therapeutically effective amount or dose can be estimated initially from in vitro assays. For example, a dose can be formulated in animal models and such information can be used to more accurately determine useful doses in humans.

Toxicity and therapeutic efficacy of the ddh- or deoxy-ddh compound or a composition thereof, as described herein, can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

The amount of a composition to be administered will, of course, be dependent on e.g., the subject being treated, the severity of the affliction, the manner of administration, the judgment of the prescribing physician, etc.

An effective amount can be administered to a subject in one or more doses. In terms of treatment, an effective amount is an amount that is sufficient to treat a disease in a subject, for example but not limited to a viral infection, a disease associated with a viral infection, or a cancer associated with a viral infection. In some embodiments, an effective amount is an amount that is sufficient to inhibit viral replication, wherein said virus comprises an RNA virus or a DNA virus, or a single stranded virus or a double stranded virus, and any combination thereof. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the ddh- or deoxy-ddh compound or a pharmaceutically acceptable salt thereof or a composition thereof, being administered.

Compounds represented by the structure of Formulas I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16 or a pharmaceutically acceptable salt thereof, as disclosed herein in detail, or pharmaceutical compositions thereof, may be administered by a variety of different routes. In some embodiments, a ddh- or deoxy-ddh compound or a composition thereof, is administered orally, intravenously, intraperitoneally, or subcutaneously.

For oral preparations, a ddh- or deoxy-ddh compound or a pharmaceutically acceptable salt thereof or a composition thereof, may be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A ddh- or deoxy-ddh compound or a composition thereof, may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

A ddh- or deoxy-ddh compound or a pharmaceutically acceptable salt thereof or a composition thereof, may be utilized in aerosol formulation to be administered via inhalation. The compounds represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, disclosed herein may be formulated into pressurized acceptable propellants such as dichlorodifluoromethane, propane, nitrogen and the like.

Furthermore, a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, may be made into suppositories by mixing with a variety of bases such as emulsifying bases or water-soluble bases. A compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VIII, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, may be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise a ddh- or deoxy-ddh compound or a composition thereof, in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for the active agents depend on the particular compound employed and the effect to be achieved, and the pharmacodynamics associated with each compound in the host.

Other modes of administration will also find use with treating subjects in need of treating a disease in a subject in need, for example but not limited to treating a viral infection, a disease associated with a viral infection, or a cancer associated with a viral infection. For instance, a compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, may be formulated in suppositories and, in some cases, aerosol and intranasal compositions. For suppositories, vehicle composition will include traditional binders and carriers such as, polyalkylene glycols, or triglycerides. Such suppositories may be formed from mixtures containing the active ingredient in the range of about 0.5% to about 10% (w/w), or about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the ddh- or deoxy-ddh compound or a composition thereof by the nasal mucosa.

A compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, may be administered as injectables. Typically, injectable compositions are prepared as liquid solutions or suspensions; solid forms suitable for solution in, or suspension in, liquid vehicles prior to injection may also be prepared. The preparation may also be emulsified, or the compound represented by the structure of Formula I, I1, I2, I3, I4, I5, I6, II, II1, II2, II3, II4, II5, II6, II7, II8, II9, II10, II11, II12, II13, II14, II15, II16, II17, II18, II19, II20, II21, II22, II23, III, III1, III2, III3, III4, IV, IV1, IV2, IV3, IV4, IV5, IV6, V, V1, V2, V3, V4, V5, V6, V7, V8, V9, V10, V11, V12, V13, V14, V15, V16, V17, V18, V19, V20, V21, V22, V23, VI, VI1, VI2, VI3, VI4, VII, VII1, VII2, VII3, VII4, VII5, VII6, VIII, VIII1, VIII2, VIII3, VIII4, VIII5, VIII6, VIII7, VIII8, VIII9, VIII10, VIII11, VIII12, VIII13, VIII14, VIII15, VIII16, VIII17, VIII18, VIII19, VIII20, VIII21, VIII22, VIII23, IX, IX1, IX2, IX3, IX4, X, X1, X2, X3, X4, 14, 15 16, A14, A15, or A16, or a pharmaceutically acceptable salt thereof, as disclosed herein in detail, or pharmaceutical compositions thereof, may encapsulated in liposome vehicles.

In some embodiments, the methods described herein comprise the ddh- or a deoxy-ddh-compounds as disclosed herein in detail.

In some embodiments, the pharmaceutical compositions provided herein, comprises the ddh- or a deoxy-ddh-compounds as disclosed herein in detail.

In some embodiments, the use of the pharmaceutical composition provided herein comprises use of the ddh- or a deoxy-ddh-compounds as disclosed herein in detail.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

A skilled artisan would appreciate that the singular form “a”, “an” and “the” include plural references unless the context clearly dictates otherwise. For example, the term “an agent” or “at least an agent” may include a plurality of agents, including mixtures thereof.

In some embodiment, “treating” comprises therapeutic treatment and “preventing” comprises prophylactic or preventative measures, wherein the object is to prevent or lessen the targeted pathologic condition or disorder as described hereinabove. Thus, in some embodiments, treating may include directly affecting a viral infection or a disease associated with a viral infection including cancer. In some embodiments, “preventing” encompasses inter alia, delaying the onset of symptoms, preventing relapse to a disease, decreasing the number or frequency of relapse episodes, increasing latency between symptomatic episodes, or a combination thereof.

A skilled artisan would appreciate that the term “treatment” may encompass clinical intervention in an attempt to alter a disease course of the individual being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease, a treatment can prevent worsening of a disease in an affected or diagnosed subject or a subject suspected of having the disease, for example a subject infected with a virus, but not yet demonstrating any symptoms. In some embodiments, administration for treatment may prevent the onset of a disease or a symptom of the disease in a subject at risk for the disease or suspected of having the disease, for example but not limited to a subject infected with a virus but showing no symptoms of disease.

A skilled artisan would appreciate that the term “subject” may encompass a vertebrate, in some embodiments, to a mammal, and in some embodiments, to a human.

A skilled artisan would appreciate that the term “effective amount” may encompass an amount sufficient to have a therapeutic effect. In some embodiments, an “effective amount” is an amount sufficient to treat a disease or a symptom thereof in a subject in need, reduce or inhibit the progression of a disease, ameliorate or alleviate suffering from the disease, reduce or inhibit the spread of the disease, or any combination thereof.

Throughout this application, various embodiments disclosed herein may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

### EXAMPLES

Reference is now made to the following examples, which together with the above descriptions illustrate some embodiments in a non-limiting fashion.

### Example 1—Preparation of Compound 2

Compound 2 is prepared as described in Scheme 15:

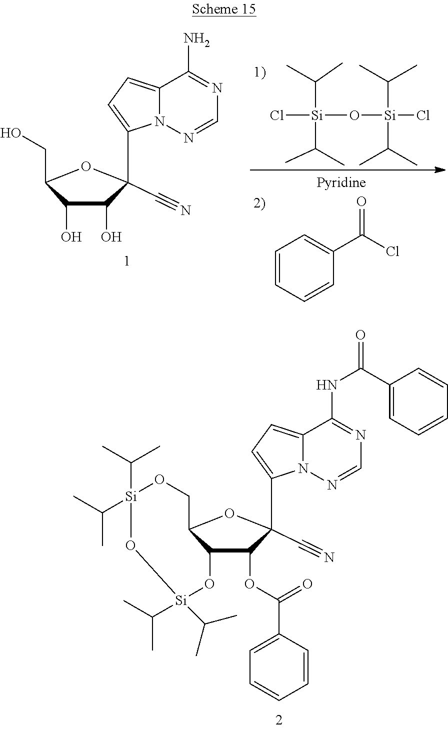

Compound 2 is prepared by protecting the OH group of C3′ and C′5, followed by protecting C2′ with a benzoyl group.

### Example 2—Preparation of Compound 3

Compound 3 is prepared as described in Scheme 16:

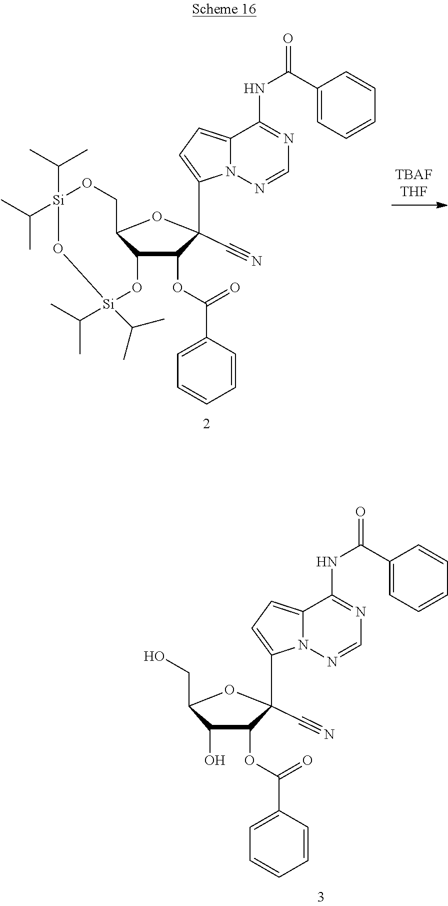

Compound 3 is prepared by reacting compound 2 with TBAF to selectively deprotect positions C3′ and C5′.

### Example 3—Preparation of Compound 4

Compound 4 is prepared as described in Scheme 17:

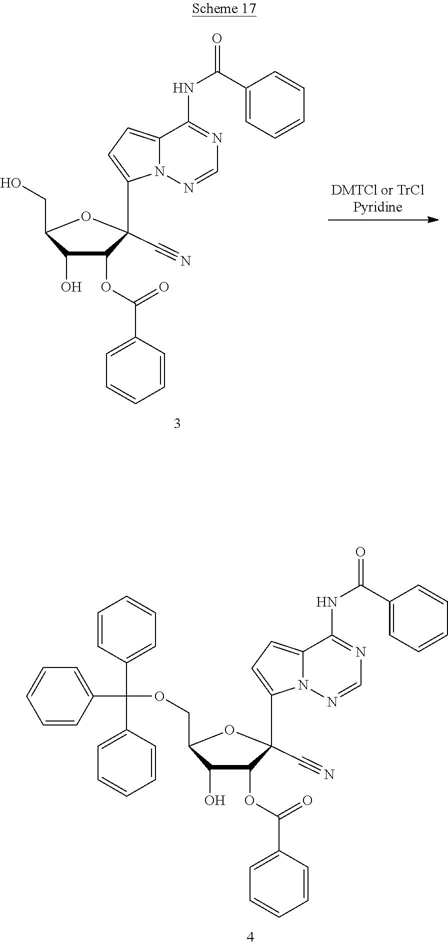

Compound 4 is prepared by reacting compound 3 with DMTCl (4,4′-Dimethoxytrityl chloride) or TrCl (trityl chloride), in the present of pyridine to obtain the C3′ protected compound 4.

### Example 4—Preparation of Compound 5

Compound 5 is prepared as described in Scheme 18:

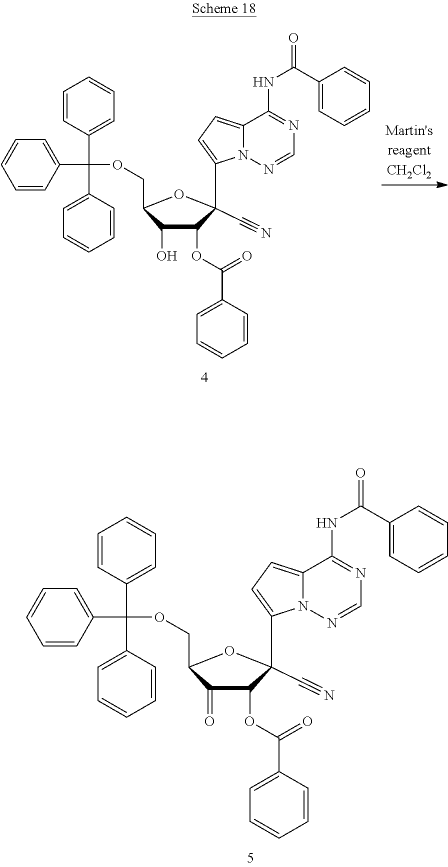

Compound 5 is prepared by oxidizing compound 4 at position C3′.

### Example 5—Preparation of Compound 6

Compound 6 is prepared as described in Scheme 19:

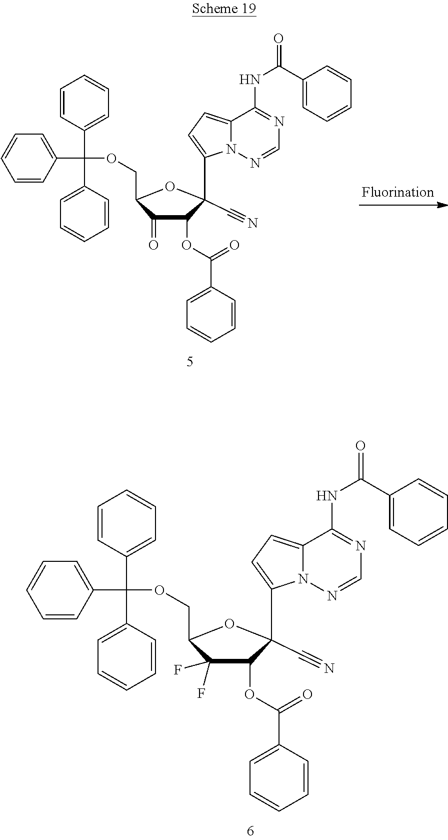 [SMILES: N#C[C@@]1(c2ccc3c(NC(=O)c4ccccc4)ncnn23)O[C@H](COC(c2ccccc2)(c2ccccc2)c2ccccc2)C(=O)[C@@H]1OC(=O)c1ccccc1 ; N#C[C@@]1(c2ccc3c(NC(=O)c4ccccc4)ncnn23)O[C@H](COC(c2ccccc2)(c2ccccc2)c2ccccc2)C(F)(F)[C@@H]1OC(=O)c1ccccc1]

Compound 6 is prepared by fluorinating compound 5 at position C3′-reacting compound 5 with DAST reagent (Diethylaminosulfur trifluoride).

### Example 6—Preparation of Compound 7

Compound 7 is prepared as described in Scheme 20:

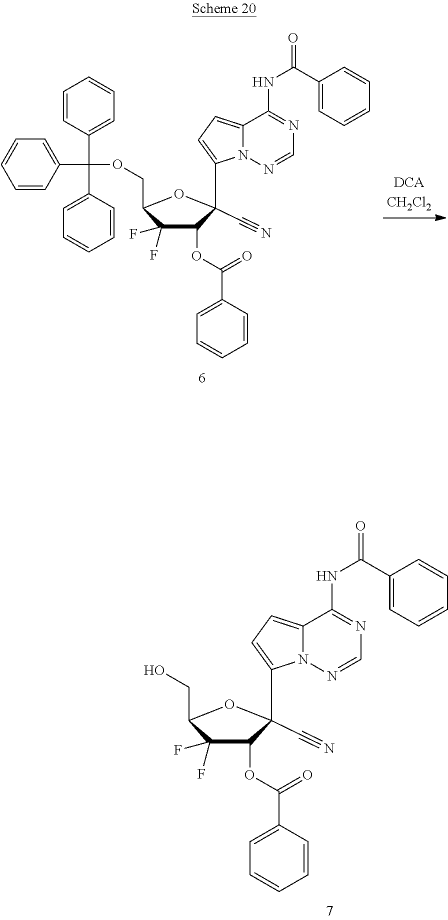 [SMILES: N#C[C@@]1(c2ccc3c(NC(=O)c4ccccc4)ncnn23)O[C@H](COC(c2ccccc2)(c2ccccc2)c2ccccc2)C(F)(F)[C@@H]1OC(=O)c1ccccc1 ; N#C[C@@]1(c2ccc3c(NC(=O)c4ccccc4)ncnn23)O[C@H](CO)C(F)(F)[C@@H]1OC(=O)c1ccccc1]

Compound 7 is prepared by reacting compound 6 with an acid (DCA—dichloroacetic acid) for the removal of the trityl group.

### Example 7—Preparation of Compound 8

Compound 8 is prepared as described in Scheme 21:

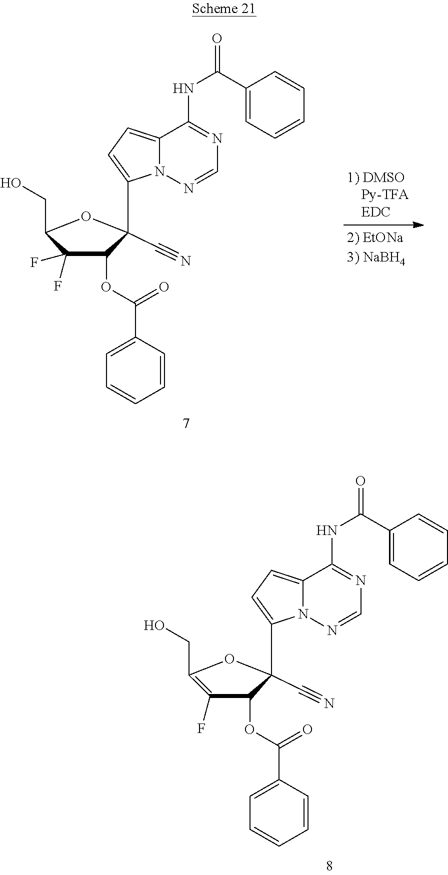 [SMILES: N#C[C@@]1(c2ccc3c(NC(=O)c4ccccc4)ncnn23)O[C@H](CO)C(F)(F)[C@@H]1OC(=O)c1ccccc1 ; N#C[C@@]1(c2ccc3c(NC(=O)c4ccccc4)ncnn23)OC(CO)=C(F)[C@@H]1OC(=O)c1ccccc1]

Compound 8 is prepared by reacting compound 7 with an oxidizing agent, followed by a base for elimination reaction, and a reducing agent.

### Example 8—Preparation of Compound 16

Compound 16 is prepared as described in Scheme 22:

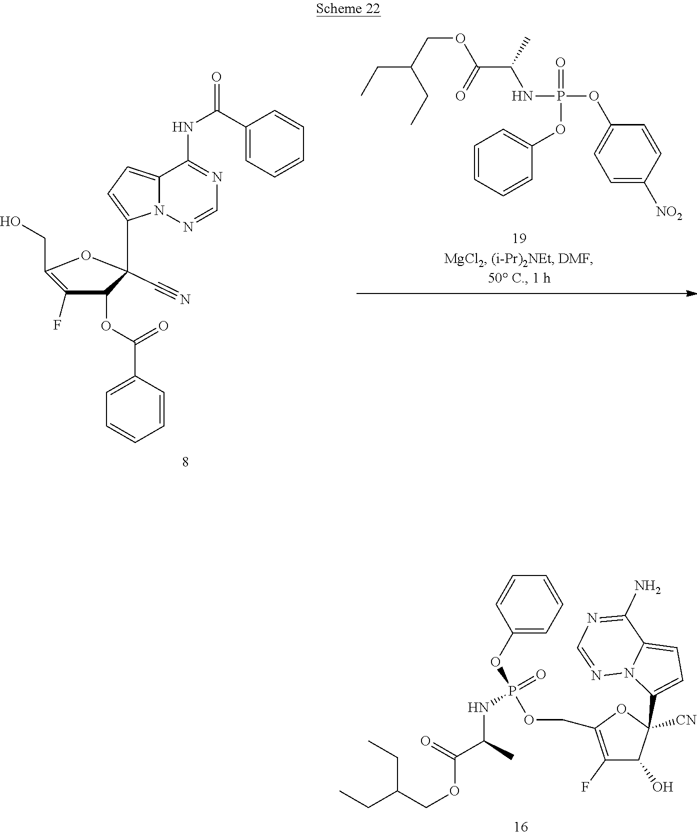 [SMILES: N#C[C@@]1(c2ccc3c(NC(=O)c4ccccc4)ncnn23)OC(CO)=C(F)[C@@H]1OC(=O)c1ccccc1 ; CCC(CC)COC(=O)[C@H](C)NP(=O)(Oc1ccccc1)Oc1ccc([N+](=O)[O-])cc1 ; CCC(CC)COC(=O)[C@H](C)N[P@](=O)(OCC1=C(F)[C@@H](O)[C@](C#N)(c2ccc3c(N)ncnn23)O1)Oc1ccccc1]

Compound 16 is prepared by reacting compound 8 with Compound 19.

### Example 9—Preparation of Compounds 9a and 9b

Compounds 9a and 9b are prepared as described in Scheme 23:

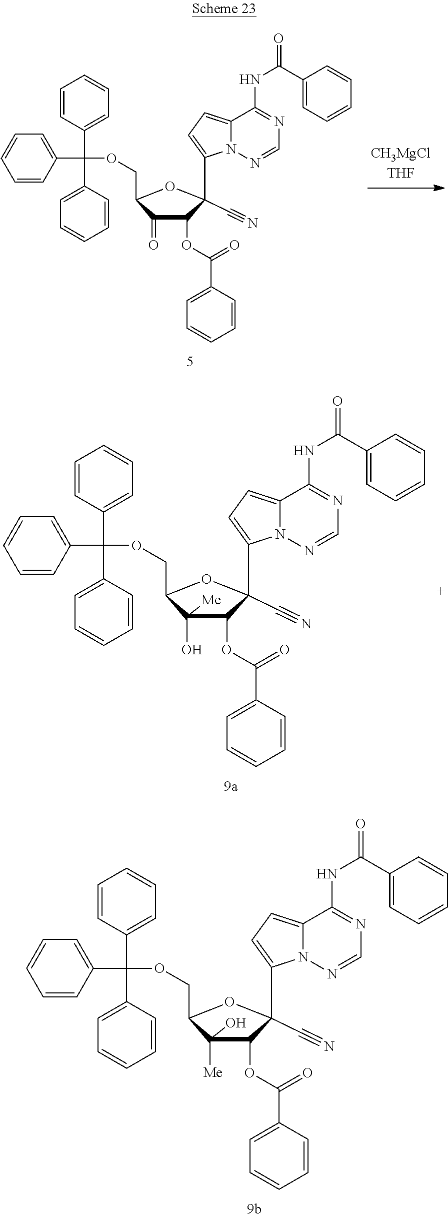

Compounds 9a and 9b are prepared by reacting compound 5 with a Grignard reagent and the like to introduce the alkyl group at C3′.

### Example 10—Preparation of Compounds 10a and 10b

Compounds 10a and 10b are prepared as described in Scheme 24:

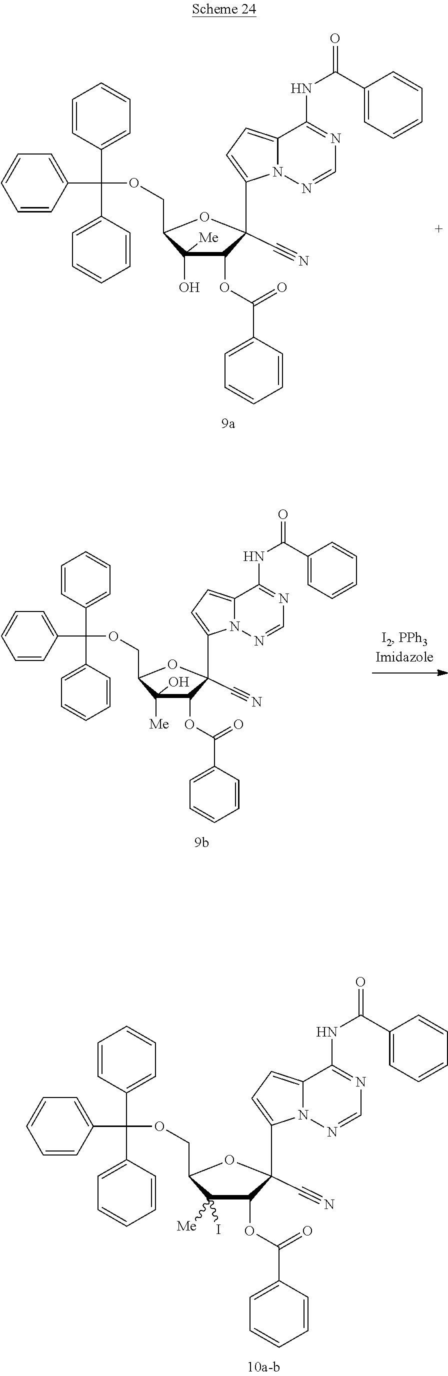

Compounds 10a and 10b are prepared by reacting compounds 9a and 9b with a Grignard reagent and the like to introduce the alkyl group at C3′.

### Example 11—Preparation of Compounds Ha and 11b

Compounds 11a and 11b are prepared as described in Scheme 25:

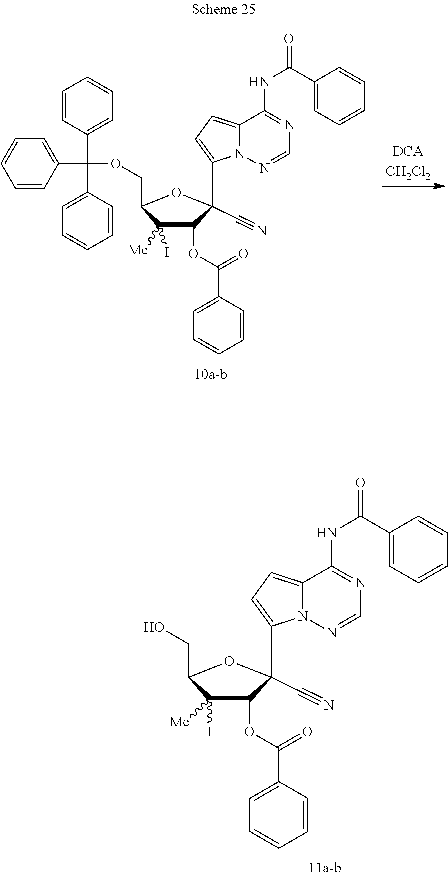

Compounds 11a and 11b are prepared by reacting compounds 10a and 10b with an acid (DCA—dichloroacetic acid) for the removal of the trityl group

### Example 12—Preparation of Compound 13

Compound 13 is prepared as described in Scheme 26:

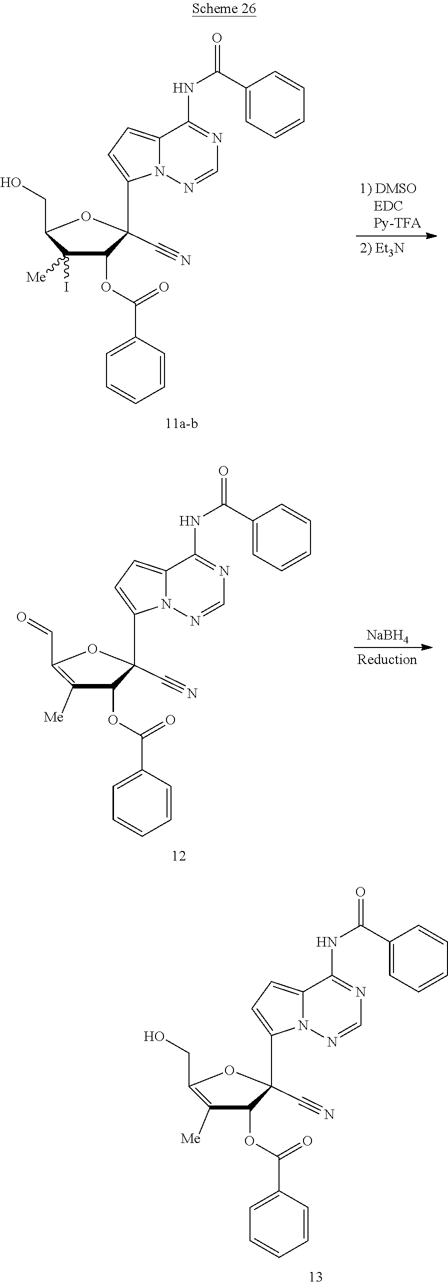

Compound 13 is prepared by reacting compounds 11a-b with an oxidizing agent followed by reacting with a base to obtain compound 12, which is further reacting with reducing agent to obtain compound 13.

### Example 13—Preparation of Compound 14

Compound 14 is prepared as described in Scheme 27:

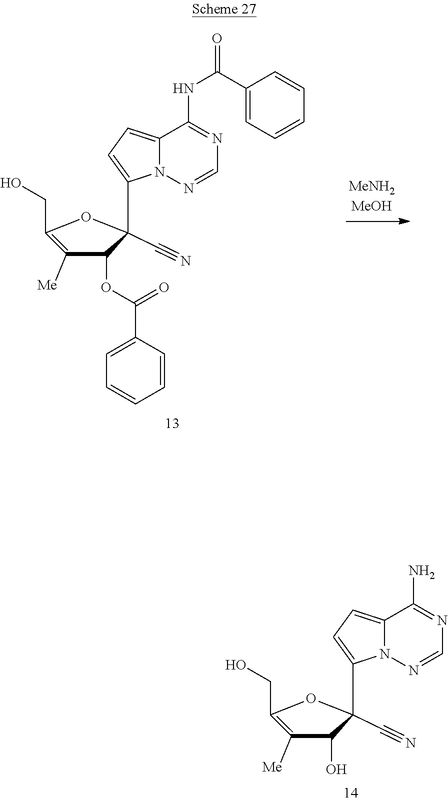 [SMILES: CC1=C(CO)O[C@@](C#N)(c2ccc3c(NC(=O)c4ccccc4)ncnn23)[C@H]1OC(=O)c1ccccc1 ; CC1=C(CO)O[C@@](C#N)(c2ccc3c(N)ncnn23)[C@H]1O]

Compound 14 is prepared by de-protecting compounds 13.

### Example 14—Preparation of Compound 14

Compound 15 is prepared as described in Scheme 28:

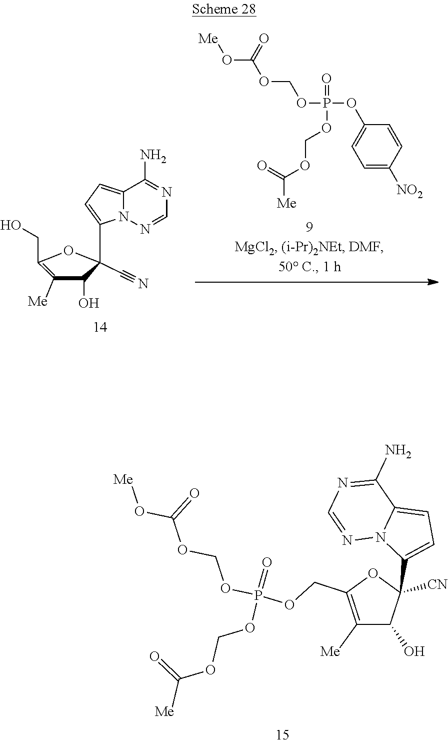 [SMILES: CC1=C(CO)O[C@@](C#N)(c2ccc3c(N)ncnn23)[C@H]1O ; COC(=O)OCOP(=O)(OCOC(C)=O)Oc1ccc([N+](=O)[O-])cc1 ; COC(=O)OCOP(=O)(OCOC(C)=O)OCC1=C(C)[C@@H](O)[C@](C#N)(c2ccc3c(N)ncnn23)O1]

Compound 15 is prepared by reacting compound 14 with Compound 9 for the substitution of position C5′ of compound 14.

While certain features disclosed have been illustrated and described herein, many modifications, substitutions, changes, and equivalents will now occur to those of ordinary skill in the art. It is, therefore, to be understood that the appended claims are intended to cover all such modifications and changes as fall within the true spirit of the compounds and methods of use disclosed herein.

1. A ddh- or a deoxy-ddh-compound represented by the structure of Formula III 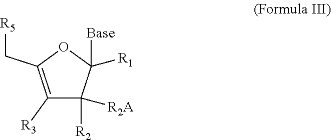
or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
wherein if R₃ is H, then R₁ is —N₃, an alkyne-R₈, or an alkyl;
R₅ is 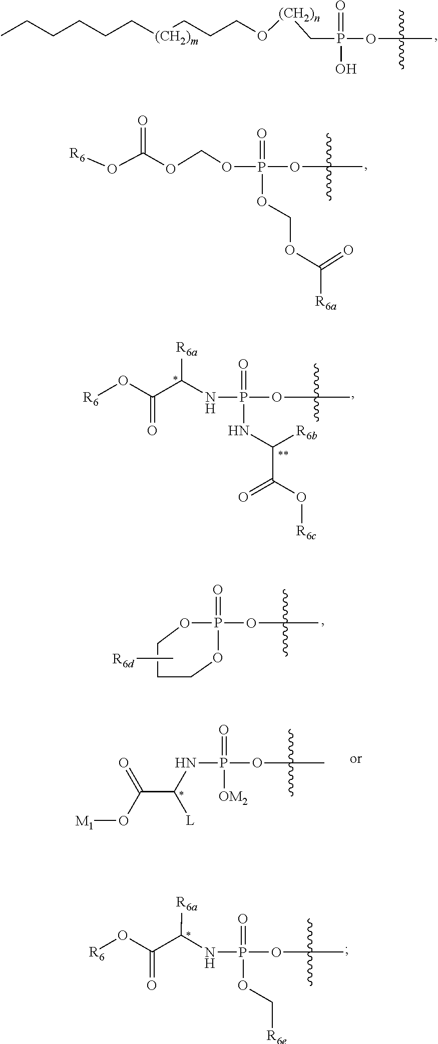
n is an integer between 1-4;
m is an integer between 1-4;
R₆, R₆ₐ, R_{6b}, and R_{6c} are each independently a branched or linear alkyl;
R_{6d} is H or an alkyl;
R₆ₑ is a haloalkyl;
R₈, R₉, and R₁₀ are each independently an alkyl;
M₁ is a branched or linear alkyl;
L is a side chain of a natural or unnatural amino acid;
M₂ is substituted or unsubstituted aryl, or substituted or unsubstituted heteroaryl; and
Base₃ is a C-linked base.

2. (canceled)

3. The compound of claim 1, wherein the C-linked base comprises:

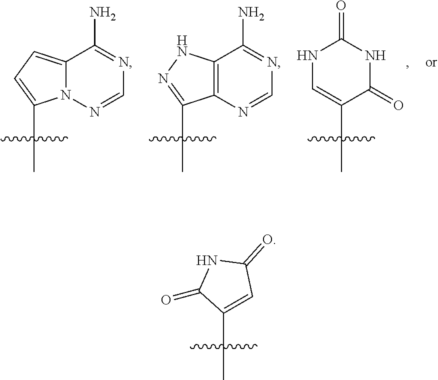

4. The ddh- or a deoxy-ddh-compound of claim 1, represented by the structure of Compound 15 or Compound 16:

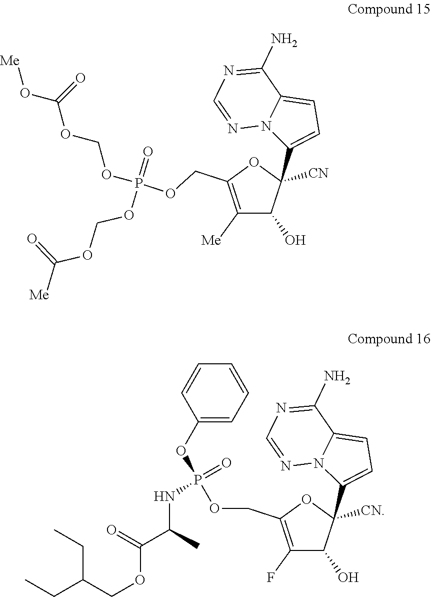 [SMILES: COC(=O)OCOP(=O)(OCOC(C)=O)OCC1=C(C)[C@@H](O)[C@](C#N)(c2ccc3c(N)ncnn23)O1 ; CCC(CC)COC(=O)[C@H](C)N[P@](=O)(OCC1=C(F)[C@@H](O)[C@](C#N)(c2ccc3c(N)ncnn23)O1)Oc1ccccc1]

5. A pharmaceutical composition comprising at least one of the compounds of claim 1, and a pharmaceutically acceptable carrier.

6. (canceled)

7. A method of treating a disease in a subject in need thereof, comprising administering the pharmaceutical composition of claim 5, wherein said disease comprises a virus-induced disease, a cancer, an autoimmune disease, an immune disorder, a bacterial associated disease or infection, or a combination thereof.

8. (canceled)

9. The method of claim 7, wherein said disease is caused by a virus selected from the group consisting of norovirus, rotavirus, rabies virus, West Nile virus, enterovirus, echovirus, coxsackievirus, herpes simplex virus (HSV), varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus (MERS-CoV), SARS coronavirus 2 (SARS-CoV-2), Epstein-Barr virus (EBV), influenza virus, influenza virus A2, influenza virus B, influenza virus A (H1N1), respiratory syncytial virus (RSV), polyoma viruses, JV virus, BK virus, Tacaribe virus, Ebola virus, Dengue virus, Pneumoviridae virus, respiratory syncytial virus, human metapneumovirus, Picornaviridae virus, human rhinovirus, Flaviviridae virus, yellow fever virus, Filoviridae virus, and hepatitis virus A, B, C, D, or E, and any combination thereof.

10. The method of claim 7, wherein said virus-induced disease is COVID19 caused by SARS coronavirus 2 (SARS-CoV-2) infection.

11. The method of claim 7, wherein said virus-induced disease is a Pneumoviridae virus infection, wherein said Pneumoviridae virus infection comprises a respiratory syncytial virus infection or a human metapneumovirus infection.

12. (canceled)

13. The method of claim 7, wherein said virus-induced disease is a Picornaviridae virus infection, wherein said Picornaviridae virus infection comprises a human rhinovirus infection.

14. (canceled)

15. The method of claim 7, wherein said virus-induced disease is a Flaviviridae virus infection, wherein said Flaviviridae virus infection comprises a dengue virus infection, a yellow fever virus infection, a West Nile virus infection, a zika virus infection, or a hepatitis C virus infection.

16. (canceled)

17. The method of claim 7, wherein said virus-induced disease is a Filoviridae virus infection, wherein said Filoviridae virus infection comprises an Ebola virus infection.

18. (canceled)

19. The method of claim 7, wherein said treating a disease terminates polynucleotide chain synthesis in a cell, said cell is a tumor cell, a cell infected by a virus, or a cell infected by a foreign DNA.

20. (canceled)

21. A method of terminating polynucleotide chain synthesis in a cell, comprising administering the pharmaceutical composition of claim 5.

22. The method of claim 21, wherein terminating polynucleotide chain synthesis increases termination of DNA chain synthesis, or increases termination of RNA chain synthesis, or a combination thereof.

23. The method of claim 21, wherein terminating polynucleotide chain synthesis confers viral resistance to said cell.

24. The method of claim 23, wherein said cell is a eukaryotic cell, said eukaryotic cell comprising a tumor cell, or is a cell infected by a virus or a foreign DNA.

25. (canceled)

26. (canceled)

27. A ddh- or a deoxy-ddh-compound represented by the structure of by the structure of Compound 15 or Compound 16:

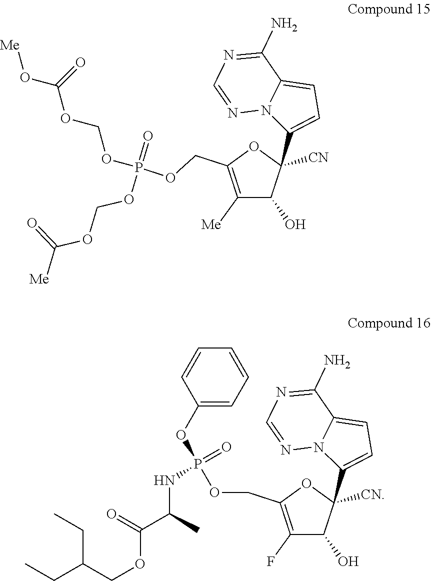 [SMILES: COC(=O)OCOP(=O)(OCOC(C)=O)OCC1=C(C)[C@@H](O)[C@](C#N)(c2ccc3c(N)ncnn23)O1 ; CCC(CC)COC(=O)[C@H](C)N[P@](=O)(OCC1=C(F)[C@@H](O)[C@](C#N)(c2ccc3c(N)ncnn23)O1)Oc1ccccc1]

28. A ddh- or a deoxy-ddh-compound represented by the structure of Formula X 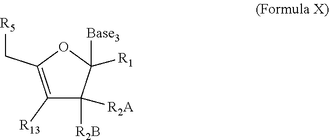
or a pharmaceutically acceptable salt thereof,
wherein
R₁ is H, —CN, —N₃, an alkyne-R₈, or an alkyl;
R₂A is H, OH, an alkyl, a halo, an alkyne-R₉, or O—C(O)OR₉;
R₂B is H, OH, an alkyl, a halo, an alkyne-R₁₀, or O—C(O)OR₁₀;
R₃ is H, a halo, or an alkyl;
R₇ is OH, 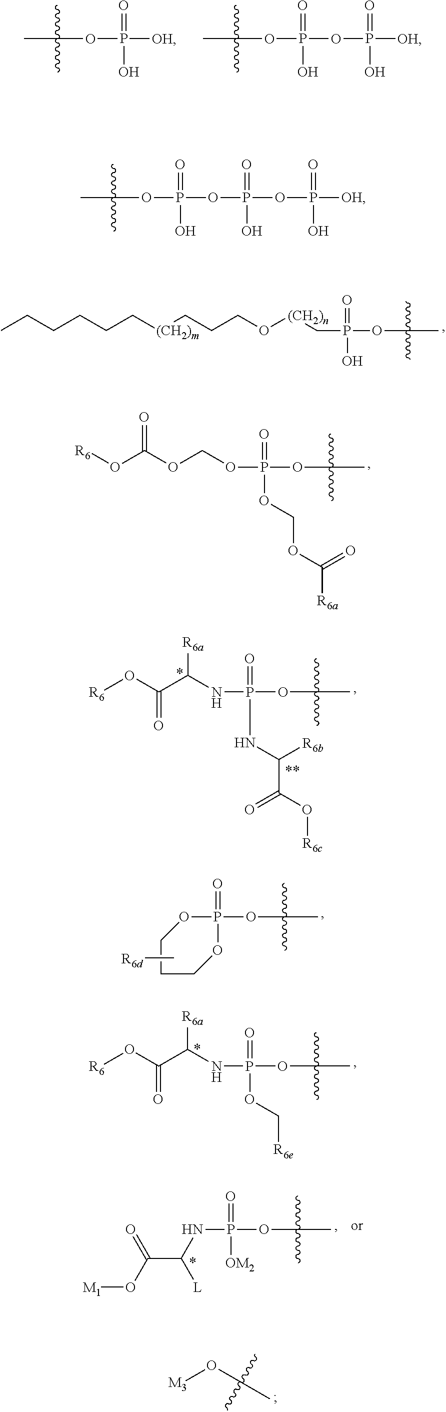
M₁ is a branched or linear alkyl;
L is a side chain of a natural or unnatural amino acid;
M₂ is substituted or unsubstituted aryl, or substituted or unsubstituted heteroaryl;
M₃ is 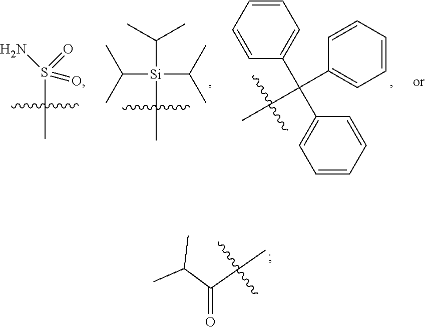
n is an integer between 1-4;
m is an integer between 1-4;
R₆, R₆ₐ, R_{6b}, and R₆ₑ are each independently a branched or linear alkyl;
R_{6d} is H or an alkyl;
R₆ₑ is a haloalkyl;
R₈, R₉, and R₁₀ are each independently an alkyl; and
Base₃ is a C-linked base.

29. A pharmaceutical composition comprising at least one of the compounds of claim 28, and a pharmaceutically acceptable carrier.

30. A method of treating a disease in a subject in need thereof, comprising administering the pharmaceutical composition of claim 29.
